(19) United States
(12) Patent Application Publication
(10) Pub. No.: US 2023/0287361 A1
(43) Pub. Date: Sep. 14, 2023

(54) ENGINEERED MONOAMINE OXIDASES FOR THE PREPARATION OF STEREOMERICALLY PURE FUSED BICYCLIC PROLINE COMPOUNDS

(71) Applicant: Codexis, Inc., Redwood City, CA (US)

(72) Inventors: Jessica Anna Hurtak, Mountain View, CA (US); Jonathan Vroom, South San Francisco, CA (US); David Watts, San Mateo, CA (US)

(21) Appl. No.: 18/181,760

(22) Filed: Mar. 10, 2023

Related U.S. Application Data
(60) Provisional application No. 63/319,135, filed on Mar. 11, 2022.

(51) Int. Cl. C12N 9/06; C12P 17/18

(52) U.S. Cl. CPC C12N 9/0022; C12Y 104/03004; C12P 17/18

(57) ABSTRACT

The present invention provides novel biocatalysts and associated methods of use for the oxidative desymmetrization of fused bicyclic proline analogues to produce APIs and intermediates. The novel biocatalysts of the present disclosure are engineered monoamine oxidase enzymes with improved solubility, thermostability, and activity on fused bicyclic proline compounds, as compared to a reference monoamine oxidase. In particular, the engineered monoamine oxidase enzymes of the present disclosure require reduced enzyme loading for the manufacturing of APIs and intermediates, as compared to a reference monoamine oxidase enzyme.

The present application claims priority to U.S. Prov. Pat. Appln. Ser. No. 63/319,135, filed Mar. 11, 2022, which is hereby incorporated by reference in its entirety for all purposes.

### TECHNICAL FIELD

The present disclosure relates to engineered monoamine oxidase enzymes and biocatalytic processes for the preparation of substantially stereomerically pure fused bicyclic proline compounds.

### REFERENCE TO SEQUENCE LISTING, TABLE OR COMPUTER PROGRAM

The official copy of the Sequence Listing is submitted concurrently with the specification as a XML file, with a file name of “CX2-228US1 ST26.xml”, a creation date of Mar. 8, 2023, and a size of 2.32 megabytes. The Sequence Listing filed is part of the specification and is incorporated in its entirety by reference herein.

### BACKGROUND

Bicyclic proline analogues are used in the discovery and development of peptidomimetic drugs. (A. Trabocchi et al. (2008) Amino Acids (2008) 34: 1-24). The hepatitis C virus protease inhibitor boceprevir (SCH 505034; ((1R,2S,5S)-N-(4-amino-1-cyclobutyl-3,4-dioxobutan-2-yl)-3-((S)-2-(3-tert-_{butylureido})-3,3-dimethylbutanoyl)-6,6-dimethyl-3-azabicyclo[3.1.0]hexane-2-carboxamide) (Malcolm et al. (2006) Antimicrob. Agents Chemother. 50(3): 1013 20) is shown below:

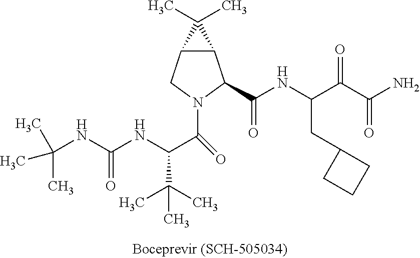 [SMILES: CC(C)(C)NC(=O)N[C@H](C(=O)N1C[C@H]2[C@@H]([C@H]1C(=O)NC(CC1CCC1)C(=O)C(N)=O)C2(C)C)C(C)(C)C]

Boceprevir is prepared from the ester of the cis-fused bicyclic L-proline analogue (1R,2S,5S)-6,6-dimethyl-3-azabicyclo[3.1.0]hexane-2-carboxylic acid, which is shown below:

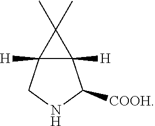

WO 2000/20824 and WO 2002/18369 describe numerous other hepatitis C protease inhibitors incorporating various fused bicyclic L-proline analogues.

Recently, the global SARS-CoV-2 (Covid-19) pandemic has inspired evaluations of the efficacy of existing peptidomimetic and small molecule protease inhibitors, including boceprevir, against the main coronavirus protease, M^{Pro} (Citarella et al. (2021) Biomolecules 2021:11(4:607). Various fused bicyclic L-proline analogues have been investigated for anti-viral activity against Covid-19 in a mouse model (Qiao et al. (2021) Science. 2021 Mar. 26; 371(6536):1374-1378). Indeed, a recent review of prospective coronavirus M^{Pro} inhibitors identified numerous fused bicyclic L-proline analogues among the most potent covalent inhibitors (Macip et al. (2021) Int J Mol Sci. 2021; 23(1):259). Thus, fused bicyclic L-proline analogues demonstrate potential for effective anti-viral activity across a spectrum of viral pathogens.

Therefore, efficient methods to synthesize fused bicyclic L-proline analogues are necessary to enable the development of novel compounds and treatments for existing and emerging viral threats.

Although the synthesis of such complex molecules using the methods and tools of organic chemistry have been reported; these syntheses generally are multi-step, intricate, expensive, and inefficient, processes of low overall yield.

Wu et al. (WO 2007/075790) discloses the production of the methyl ester of the bicyclic proline analogue (1R,2S,5S)-6,6-dimethyl-3-azabicyclo[3.1.0]hexane-2-carboxylic acid from the corresponding symmetrical (achiral) bicyclic amine of structural formula (1R,5S)-6,6-dimethyl-3-azabicyclo[3.1.0]hexane, beginning with its oxidation to the corresponding racemic imine of structural formula

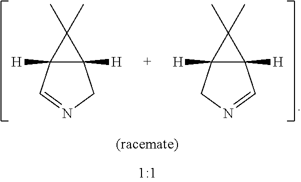

The racemic imine is subsequently reacted with cyanide to provide the racemic aminonitrile of the following structural formula

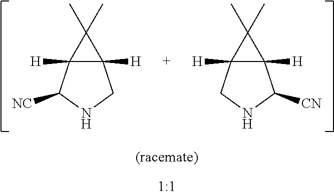

which is then reacted with acid and methanol to give the racemic amino acid methyl ester of the following structural formulae

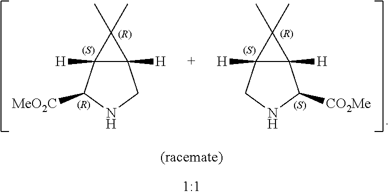

Finally, these (1R,2S, 5S) (undesired) and (1S,2R, 5R) (desired) stereoisomeric methyl esters are separated by diastereomeric salt resolution, forming either the di-p-tolyl-D-tartaric acid salt with the former enantiomer or the di-p-tolyl-L-tartaric tartaric acid salt with the latter enantiomer.

Tanoury et al. (WO 2007/022459) disclose the synthesis of racemic (t-butoxycarbonyl)octahydrocyclopenta[c]pyrrole-1-carboxylic acid from the corresponding symmetrical (achiral) bicyclic amine by making the N-Boc derivative, and reacting it with the pyrophoric agent, sec-butyllithium in the presence of more than a stoichiometric amount of a bulky diamine chelate, then carbon dioxide, all at below −70° C. to produce the racemic N-Boc amino acids depicted below:

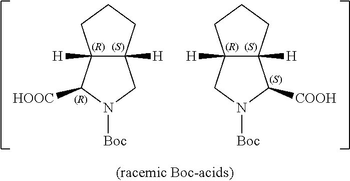

The (1R,2S, 5S) (undesired) and (1S,2R, 5R) (desired) stereoisomers of these racemic Boc-acids are then separated by diastereomeric salt resolution using single enantiomer chiral bases such as S-1-aminotetralin.

Although the desired stereoisomer of the amino acid derivative is obtained by these methods, the resolution of a mixture of enantiomers of these bicyclic proline analogues inherently involves the waste of at least one half of all of the material (e.g. raw materials, reagents, solvents, catalysts) used in the production of the racemic mixture.

Monoamine oxidase enzymes have been used to resolve and deracemize racemic chiral amines via the stereospecific oxidation of one enantiomer to the corresponding imine using oxygen. Derivatives of the flavin dependent monoamine oxidase of Aspergillus niger (MAO N) (Shilling et al. (1995) Biochim. Biophys. Acta. 1243: 529 37) have been reported as useful, in combination with non-specific chemical reducing agents, for the deracemization of (d/1) a methylbenzylamine to provide enantiomerically pure (93% ee) (d) α methylbenzylamine (Alexeeva et al. (2002), Angew. Chem. Int. Ed. 41: 3177-3180). Derivatives of the flavin dependent monoamine oxidase of Aspergillus niger were also used for deracemization of (R/S)-2-phenypyrrolidine to provide enantiomerically pure (98% ee) (R)-2-phenypyrrolidine (Carr et al. (2005), ChemBioChem 6: 637 39; Gotor et al. “Enantioselective Enzymatic Desymmetrization in Organic Synthesis,” Chem. Rev. (2005) 105: 313-54).

Previously, an efficient, chemoenzymatic process for manufacture of the boceprevir bicyclic [3.1.0] proline intermediate using engineered monoamine oxidases was reported. Monoamine oxidases derived from A. niger and A. oryzae were engineered to improve solubility, thermostability, and activity on the 6,6-dimethyl-3-azabicyclo[3.1.0]hexane substrate (Li et al. (2012), J. Am. Chem. Soc.134, 6467-6472; WO2010008828). The optimized process then proceeded via sulfonation to a chiral aminosulfonate and cyanation to a trans-nitrile prior to transformation to the methyl ester under Pinner conditions (Li et al.)

However, production of large quantities of substantially-enantiomerically pure chiral compounds is limited by the enzyme loading required for synthesis. Therefore, it necessary to provide improved engineered enzymes with reduced enzyme loading (wt/wt) requirements for the manufacturing of APIs and intermediates.

### SUMMARY

The present invention provides novel biocatalysts and associated methods of use for the enzymatic oxidative desymmetrization of 6,6-dimethyl-3-azabicyclo[3.1.0]hexane and related bicyclic proline analogues to produce APIs and intermediates, including peptidomimetic antiviral compounds. The monoamine oxidase biocatalysts of the present disclosure are engineered polypeptide variants of a previously engineered monoamine oxidase (SEQ ID NO: 6) derived from a fusion polypeptide of Aspergillus niger (SEQ ID NO: 2) and Aspergillus oryzae (SEQ ID NO: 4) monoamine oxidase homologs. These engineered monoamine oxidase polypeptides are capable of catalyzing the conversion of 6,6-dimethyl-3-azabicyclo[3.1.0]hexane and related bicyclic proline analogs to (1R,5S)-6,6-dimethyl-3-azabicyclo[3.1.0]hex-2-ene and related compounds.

The present invention provides an engineered polypeptide comprising an amino acid sequence with at least 80%, 85%, 90%, 91%, 92%, 93%, 94%, 95%, 96%, 97%, 98%, 99% sequence identity to a reference sequence of SEQ ID NO: 6, comprising at least one substitution or one substitution set at one or more positions selected from 13, 32/65/135/246/284, 32/135/284, 32/284, 47/197, 55/347/348, 61, 65/135, 65/190, 65/246, 65/284, 79, 80, 82, 85, 87, 87/185, 93, 100, 100/144/421/447, 100/144/447, 104, 107, 113, 114, 115, 117, 127, 135, 136, 144, 144/447, 145, 146, 147, 149, 149/157, 153, 155, 161, 162, 166, 167, 169, 170, 174, 177, 181, 184, 185, 187/242, 193, 196, 198, 199, 208, 211, 218, 219, 219/332, 221, 229, 232, 234, 238, 239, 240, 242, 246, 250, 252, 254, 260, 262/266/421, 262/421/447, 266/447, 277, 277/347, 284, 290, 292, 294, 298, 300, 306, 310, 323, 332, 338, 347, 349, 359, 361, 366, 381, 385, 386, 387, 392, 393, 400, 407, 411/445, 413, 418, 421, 425, 429, 433, 434, 441, 444, 445, 447, 449, 452, 475, and 483, wherein the positions are numbered with reference to SEQ ID NO: 6. In some additional embodiments, the engineered polypeptide comprises at least one substitution or one substitution set selected from 13G, 32G/65V/135Q/246M/284D, 32G/135Q/284D, 32G/284D, 47A/197N, 55A/347D/348K, 61C, 65V/135Q, 65V/190D, 65V/246M, 65V/284D, 79A, 79D, 79Q, 79S, 79V, 79Y, 80H, 80I, 80N, 80T, 80V, 82F, 82L, 82R, 82W, 85D, 85S, 87G, 87H, 87L, 87Q/185F, 87S, 87W, 87Y, 93G, 100P, 100P/144E/421S/447A, 100P/144E/447A, 104K, 107W, 113G, 113W, 114G, 115M, 117A, 117C, 117I, 117K, 117L, 117R, 117S, 117T, 127D, 127G, 127R, 135G, 136R, 144E/447A, 144M, 145G, 145L, 145S, 146A, 146F, 147A, 149M, 149M/157A, 149V, 153T, 155C, 155L, 155Q, 155R, 161D, 161L, 161R, 161Y, 162Q, 166E, 166G, 166L, 167C, 169G, 170K, 174G, 174S, 174T, 174V, 177G, 177R, 177W, 181C, 181G, 181L, 181P, 184F, 184R, 185G, 185K, 185L, 185R, 187P/242I, 193A, 196G, 196M, 198D, 199I, 199T, 199V, 208S, 211G, 218G, 219P/332L, 219S, 221C, 221R, 221V, 229E, 232T, 234V, 238K, 238R, 239G, 240A, 242I, 246M, 250V, 252A, 252E, 252G, 252L, 254A, 260F, 260S, 260V, 262F/266L/421S, 262F/421S/447A, 266L/447A, 277T, 277T/347D, 284D, 290Q, 290Y, 292N, 294V, 298C, 300R, 306V, 3105, 323R, 332G, 332L, 332S, 338A, 338G, 338S, 347D, 347N, 349E, 359S, 361L, 366G, 381A, 385G, 386E, 386G, 387I, 387Q, 387W, 392G, 392S, 393G, 393K, 393M, 400D, 407G, 411V/445R, 413R, 418A, 418G, 4215, 425L, 429G, 433N, 433S, 434E, 441H, 444D, 444L, 444R, 444S, 445D, 445R, 447A, 449R, 449S, 452M, 475E, 475I, 475L, 4755, and 483R, wherein the positions are numbered with reference to SEQ ID NO: 6. In some embodiments, the engineered polypeptide comprises at least one substitution or one substitution set selected from T13G, E32G/T65V/T135Q/I246M/N284D, E32G/T135Q/N284D, E32G/N284D, G47A/D197N, T55A/C347D/P348K, A61C, T65V/T135Q, T65V/E190D, T65V/I246M, T65V/N284D, W79A, W79D, W79Q, W79S, W79V, W79Y, S80H, S80I, S80N, S80T, S80V, N82F, N82L, N82R, N82W, G85D, G85S, P87G, P87H, P87L, P87Q/E185F, P87S, P87W, P87Y, T93G, S100P, S100P/A144E/C421S/C447A, S100P/A144E/C447A, R104K, T107W, N113G, N113W, A114G, L115M, P117A, P117C, P117I, P117K, P117L, P117R, P117S, P117T, H127D, H127G, H127R, T135G, T136R, A144E/C447A, A144M, E145G, E145L, E145S, D146A, D146F, E147A, L149M, L149M/T157A, L149V, L153T, K155C, K155L, K155Q, K155R, G161D, G161L, G161R, G161Y, T162Q, T166E, T166G, T166L, V167C, P169G, F170K, M174G, M174S, M174T, M174V, V177G, V177R, V177W, R181C, R181G, R181L, R181P, D184F, D184R, E185G, E185K, E185L, E185R, S187P/M242I, D193A, R196G, R196M, E198D, L199I, L199T, L199V, E208S, I211G, T218G, L219P/I332L, L219S, N221C, N221R, N221V, H229E, A232T, S234V, Y238K, Y238R, Q239G, G240A, M242I, 1246M, F250V, D252A, D252E, D252G, D252L, Q254A, R260F, R260S, R260V, W262F/A266L/C421S, W262F/C421S/C447A, A266L/C447A, C277T, C277T/C347D, N284D, R290Q, R290Y, T292N, R294V, E298C, A300R, C306V, L310S, S323R, I332G, I332L, I332S, C338A, C338G, C338S, C347D, C347N, D349E, P359S, N361L, A366G, C381A, A385G, H386E, H386G, N387I, N387Q, N387W, E392G, E392S, E393G, E393K, E393M, M400D, P407G, D411V/L445R, K413R, H418A, H418G, C421S, F425L, A429G, A433N, A433S, P434E, S441H, E444D, E444L, E444R, E444S, L445D, L445R, C447A, H449R, H449S, V452M, T475E, T475I, T475L, T475S, and E483R, wherein the positions are numbered with reference to SEQ ID NO: 6. In some further embodiments, the engineered polypeptide comprises an amino acid sequence with at least 80% sequence identity to any even-numbered sequence set forth in SEQ ID NO: 8 to SEQ ID NO: 414.

The present invention also provides an engineered polypeptide comprising an amino acid sequence with at least 80%, 85%, 90%, 91%, 92%, 93%, 94%, 95%, 96%, 97%, 98%, 99% sequence identity to a reference sequence of SEQ ID NO: 6, comprising at least one substitution or one substitution set selected from 79, 80, 87, 146, 149, 219/332, 238, 260, 262/266/421, 338, and 429, wherein the positions are numbered with reference to SEQ ID NO: 6. In some embodiments, the engineered polypeptide comprises at least one substitution or one substitution set selected from 79D, 80V, 87G, 146A, 149M, 219P/332L, 238R, 260S, 262F/266L/421S, 338G, and 429G, wherein the positions are numbered with reference to SEQ ID NO: 6. In some further embodiments, the engineered polypeptide comprises at least one substitution or one substitution set selected from W79D, S80V, P87G, D146A, L149M, L219P/I332L, Y238R, R260S, W262F/A266L/C421S, C338G, and A429G, wherein the positions are numbered with reference to SEQ ID NO: 6.

The present invention further provides an engineered polypeptide comprising an amino acid sequence with at least 80%, 85%, 90%, 91%, 92%, 93%, 94%, 95%, 96%, 97%, 98%, 99% sequence identity to a reference sequence of SEQ ID NO: 6, comprising at least at least one substitution or one substitution set selected from 79, 79/80, 79/80/332/429, 79/93, 79/93/238/386/429, 79/93/386, 79/183/284/332, 79/238, 79/238/332/429, 79/238/429, 80, 80/183/284/332/381/386/429, 80/188/190/332/386, 80/237/332, 80/238/332, 80/238/386/429, 80/284, 80/284/386, 80/332, 80/332/381/386, 80/332/386, 80/381, 80/386, 80/386/429, 80/429, 82, 82/87, 82/87/113, 82/87/113/117/218, 82/87/113/117/219, 82/87/113/117/219/418, 82/87/113/117/260/338, 82/87/113/117/338/418, 82/87/113/421, 82/87/117, 82/87/117/218/421, 82/87/117/260/338/421, 82/87/117/338/418, 82/87/218, 82/87/218/219/338, 82/87/218/219/421, 82/87/218/338, 82/87/219, 82/87/219/421, 82/87/338, 82/87/338/418, 82/87/338/421, 82/113/218/338, 82/113/218/338/418, 82/113/219/338, 82/113/338, 82/218/219/421, 82/260/338/421, 82/338/418/421, 82/418, 87, 87/113, 87/113/117/219/338/418/421, 87/113/117/260/338/421, 87/113/117/418/421, 87/113/218/219/260/338, 87/113/219/421, 87/113/260, 87/117, 87/117/218/219, 87/117/219/310/338, 87/218, 87/218/219, 87/218/219/338, 87/218/418, 87/219, 87/219/338, 87/219/418, 87/260, 87/310, 87/310/338, 87/338, 87/418, 87/421, 93/184/429, 93/238, 93/238/284, 93/238/386, 93/238/386/429, 100, 100/143/145/146/149/198/242/347/348/445/447, 100/143/145/149/347/447, 100/145/146/149, 100/145/146/149/154/348/445, 100/145/146/149/198, 100/145/146/149/198/242/250/252/347/348/447, 100/145/146/149/205/347/445/447, 100/145/146/149/242/445, 100/145/146/149/250/277/347/348/447, 100/145/149/154/246/277, 100/145/149/198/242/246/277/348/447, 100/145/149/198/242/246/445/447, 100/145/149/198/242/277/347/348/447, 100/145/149/198/252/348/445, 100/145/149/205, 100/145/149/242/246/277/347/445, 100/145/149/242/246/347/348/445/447, 100/145/149/242/445/447, 100/145/149/246/252/277/347/445/447, 100/145/149/246/347/445, 100/145/149/277/348/445, 100/145/149/347/445/447, 100/145/198/242/246/348, 100/145/198/277/348/445, 100/145/198/300, 100/145/205/246/277/348/445, 100/145/205/277/348, 100/145/242/246/277/445/447, 100/145/246/347/348/445, 100/145/277/347, 100/145/348, 100/146/149/198/242/246/277/347/348/445/447, 100/146/198/347/348/447, 100/146/198/445, 100/146/242/277/447, 100/146/250/447, 100/146/347/348/445/447, 100/149/198, 100/149/242/246/277/347/348/445, 100/149/242/246/277/347/348/447, 100/149/242/347/348/447, 100/149/246/277/348/445, 100/149/246/447, 100/149/250/347/447, 100/198/205/348, 100/198/277/347/348, 100/198/347/348, 100/198/347/348/445, 100/198/347/445, 100/198/447, 100/242/246/277, 100/242/246/347, 100/242/277/348, 100/242/347/348/445, 100/242/347/348/445/447, 100/242/348/445, 100/246/277/348/445, 100/277/347/445/447, 100/277/348, 100/277/348/447, 100/347/348/445, 100/347/447, 100/445/447, 110/338/418, 113/218/338/421, 113/338, 113/338/421, 117/219, 117/260, 121/198/347/445/447, 145/146/149/154/246/277/347/348, 145/146/149/198/242/252/277/347, 145/146/149/198/277/445/447, 145/146/149/205/348/447, 145/146/149/250/277/347/348/447, 145/146/149/277/347, 145/146/149/347/348, 145/146/198/205/242/277/348/447, 145/146/198/445/447, 145/146/246/277/447, 145/146/246/347/348/447, 145/146/277/347, 145/146/347/447, 145/149/198/205/348, 145/149/198/242/246/277/347/447, 145/149/198/242/246/348, 145/149/198/242/277/347, 145/149/198/246/447, 145/149/198/347/447, 145/149/205/242/347/348/445, 145/149/205/246/250/252, 145/149/205/277/445, 145/149/242, 145/149/242/348, 145/149/246, 145/149/246/347/445, 145/149/246/447, 145/149/347/348/445, 145/149/347/447, 145/149/447, 145/155/445/447, 145/198/205/348, 145/246, 145/246/252/347/447, 145/250/252/348, 145/252/348/447, 145/255, 145/277/347/445, 145/277/348/447, 145/347/348/445/447/491, 145/445/447, 146/149/151/246/348, 146/149/242/246/445, 146/149/246/252/447, 146/149/277/348/447, 146/198/246/277, 146/198/277/348/447, 146/242, 146/242/246/277/347/348/445/447, 146/246/277/348/445/447, 146/277, 146/277/347/348, 146/277/347/445/447, 146/277/445/447, 146/347/348/447, 146/348, 146/445/447, 149, 149/198/277/347/348, 149/198/348, 149/198/445, 149/242/246, 149/242/246/348/445, 149/347/348/445/447, 149/445, 184, 184/284, 184/284/429, 184/386, 198/242/348, 198/246, 198/250, 198/277/348/447, 198/445, 205, 205/246/347, 205/252/347/445/447, 205/347/348/445/447, 218, 218/260/338, 219, 219/338/418, 219/421, 238, 238/242/284, 238/284/386/429, 238/332/429, 238/381/429, 238/386, 238/429, 242, 242/277/347/348, 242/284/381/429, 242/348, 242/445/447, 246, 246/277/348/447, 246/445/447, 260, 260/338, 260/338/418/421, 277/347/348/445, 277/347/447, 284, 284/332, 284/386, 284/386/429, 310/338/418, 310/418/421, 332, 332/386, 332/429, 338, 338/418, 347/348/445, 347/348/447, 348, 348/445/447, 348/447, 386, 386/429, 418, 429, and 447, wherein the positions are numbered with reference to SEQ ID NO: 6. In some additional embodiments, the engineered polypeptide comprises at least one substitution or one substitution set selected from 79A/80H, 79A/93G/238R/386E/429G, 79A/93G/386E, 79A/238R, 79A/238R/332G/429G, 79A/238R/429G, 79D, 79D/80V, 79D/80V/332G/429G, 79D/93G, 79D/183F/284D/332G, 80H, 80H/183F/284D/332G/381A/386E/429G, 80H/188F/190D/332G/386E, 80H/238R/386E/429G, 80H/332G/381A/386E, 80H/381A, 80H/386E, 80H/429G, 80V, 80V/237S/332G, 80V/238R/332G, 80V/284D, 80V/284D/386E, 80V/332G, 80V/332G/386E, 80V/386E, 80V/386E/429G, 82F, 82F/87G/113G/117K/219S, 82F/87G/113G/117K/260S/338G, 82F/87G/117K, 82F/87G/218G, 82F/87G/218G/219S/421S, 82F/87G/218G/338S, 82F/87G/338S/418G, 82F/87L, 82F/87L/113G/117K/218G, 82F/87L/113G/117K/219S/418G, 82F/87L/113G/117K/338G/418G, 82F/87L/117K, 82F/87L/219S/421S, 82F/87L/338S, 82F/113G/218G/338S/418G, 82F/418G, 82R/87G, 82R/87G/113G/421S, 82R/87G/117K/218G/421S, 82R/87G/117K/260S/338S/4215, 82R/87G/117K/338A/418G, 82R/87L, 82R/87L/113G, 82R/87L/218G/219S/338S, 82R/87L/219S, 82R/87L/338G/421S, 82R/113G/218G/338A, 82R/113G/219S/338S, 82R/113G/338S, 82R/218G/219S/421S, 82R/260S/338A/421S, 82R/338A/418G/421S, 87G, 87G/113G, 87G/113G/117K/418G/421S, 87G/113G/260S, 87G/117K/219S/310S/338S, 87G/218G/219S/338S, 87G/219S, 87G/260S, 87G/310S, 87G/310S/338S, 87G/338A, 87G/418G, 87L, 87L/113G, 87L/113G/117K/219S/338G/418G/421S, 87L/113G/117K/260S/338S/421S, 87L/113G/218G/219S/260S/338A, 87L/113G/219S/421S, 87L/117K, 87L/117K/218G/219S, 87L/218G, 87L/218G/219S, 87L/218G/418G, 87L/219S, 87L/219S/338G, 87L/219S/418G, 87L/338A, 87L/421S, 93G/184R/429G, 93G/238R, 93G/238R/284D, 93G/238R/386E, 93G/238R/386E/429G, 100P, 100P/143D/145S/146A/149M/198D/242I/347N/348K/445R/447A, 100P/143V/145G/149M/347N/447A, 100P/145G/146A/149M/250V/277T/347N/348K/447A, 100P/145G/149M/154L/246M/277T, 100P/145G/149M/198D/242I/246M/445R/447A, 100P/145G/149M/198D/252G/348K/445R, 100P/145G/149M/205T, 100P/145G/149M/242I/246M/347D/348K/445R/447A, 100P/145G/149M/242I/445R/447A, 100P/145G/198D/242I/246M/348K, 100P/145G/198D/277T/348K/445R, 100P/145G/198D/300V, 100P/145G/205T/246M/277T/348K/445R, 100P/145G/242I/246M/277T/445R/447A, 100P/145G/348K, 100P/145S/146A/149M, 100P/145S/146A/149M/154L/348K/445R, 100P/145S/146A/149M/198D, 100P/145S/146A/149M/198D/242I/250V/252G/347N/348K/447A, 100P/145S/146A/149M/205T/347D/445R/447A, 100P/145S/146A/149M/242I/445R, 100P/145S/149M/198D/242I/246M/277T/348K/447A, 100P/145S/149M/198D/242I/277T/347N/348K/447A, 100P/145S/149M/242I/246M/277T/347N/445R, 100P/145S/149M/246M/252G/277T/347N/445R/447A, 100P/145S/149M/246M/347N/445R, 100P/145S/149M/277T/348K/445R, 100P/145S/149M/347N/445R/447A, 100P/145S/205T/277T/348K, 100P/145S/246M/347N/348K/445R, 100P/145S/277T/347N, 100P/146A/149M/198D/242I/246M/277T/347D/348K/445R/447A, 100P/146A/198D/347D/348K/447A, 100P/146A/198D/445R, 100P/146A/242I/277T/447A, 100P/146A/250V/447A, 100P/146A/347D/348K/445R/447A, 100P/149M/198D, 100P/149M/242I/246M/277T/347N/348K/445R, 100P/149M/242I/246M/277T/347N/348K/447A, 100P/149M/242I/347N/348K/447A, 100P/149M/246M/277T/348K/445R, 100P/149M/246M/447A, 100P/149M/250V/347N/447A, 100P/198D/205T/348K, 100P/198D/277T/347N/348K, 100P/198D/347D/348K, 100P/198D/347D/348K/445R, 100P/198D/347N/445R, 100P/198D/447A, 100P/242I/246M/277T, 100P/242I/246M/347N, 100P/242I/277T/348K, 100P/242I/347D/348K/445R, 100P/242I/347D/348K/445R/447A, 100P/242I/348K/445R, 100P/246M/277T/348K/445R, 100P/277T/347D/445R/447A, 100P/277T/348K, 100P/277T/348K/447A, 100P/347N/348K/445R, 100P/347N/447A, 100P/445R/447A, 110N/338G/418G, 113G/218G/338S/421S, 113G/338A/421S, 113G/338G, 117K/219S, 117K/260S, 121C/198D/347N/445R/447A, 145G/146A/149M/277T/347D, 145G/146A/149M/347D/348K, 145G/146A/277T/347N, 145G/146A/347D/447A, 145G/149M/198D/347N/447A, 145G/149M/205T/242I/347N/348K/445R, 145G/149M/205T/246M/250V/252G, 145G/149M/242I/348K, 145G/149M/246M, 145G/155I/445R/447A, 145G/198D/205T/348K, 145G/250V/252G/348K, 145G/255R, 145G/277T/347N/445R, 145S/146A/149M/154L/246M/277T/347N/348K, 145S/146A/149M/198D/242I/252G/277T/347N, 145S/146A/149M/198D/277T/445R/447A, 145S/146A/149M/205T/348K/447A, 145S/146A/149M/250V/277T/347D/348K/447A, 145S/146A/198D/205T/242I/277T/348K/447A, 145S/146A/198D/445R/447A, 145S/146A/246M/277T/447A, 145S/146A/246M/347N/348K/447A, 145S/149M/198D/205T/348K, 145S/149M/198D/242I/246M/277T/347N/447A, 145S/149M/198D/242I/246M/348K, 145S/149M/198D/242I/277T/347D, 145S/149M/198D/246M/447A, 145S/149M/205T/277T/445R, 145S/149M/242I, 145S/149M/246M/347D/445R, 145S/149M/246M/447A, 145S/149M/347N/348K/445R, 145S/149M/347N/447A, 145S/149M/447A, 145S/246M, 145S/246M/252G/347D/447A, 145S/252G/348K/447A, 145S/277T/348K/447A, 145S/347D/348K/445R/447A/491A, 145S/445R/447A, 146A/149M/151C/246M/348K, 146A/149M/242I/246M/445R, 146A/149M/246M/252G/447A, 146A/149M/277T/348K/447A, 146A/198D/246M/277T, 146A/198D/277T/348K/447A, 146A/242I, 146A/242I/246M/277T/347N/348K/445R/447A, 146A/246M/277T/348K/445R/447A, 146A/277T, 146A/277T/347N/348K, 146A/277T/347N/445R/447A, 146A/277T/445R/447A, 146A/347D/348K/447A, 146A/348K, 146A/445R/447A, 149M, 149M/198D/277T/347N/348K, 149M/198D/348K, 149M/198D/445R, 149M/242I/246M, 149M/242I/246M/348K/445R, 149M/347N/348K/445R/447A, 149M/445R, 184R, 184R/284D, 184R/284D/429G, 184R/386E, 198D/242I/348K, 198D/246M, 198D/250V, 198D/277T/348K/447A, 198D/445R, 205T, 205T/246M/347N, 205T/252G/347D/445R/447A, 205T/347D/348K/445R/447A, 218G, 218G/260S/338A, 219S, 219S/338A/418G, 219S/421S, 238R, 238R/242L/284D, 238R/284D/386E/429G, 238R/332G/429G, 238R/381A/429G, 238R/386E, 238R/429G, 242I, 242I/277T/347D/348K, 242I/348K, 242I/445R/447A, 242L/284D/381A/429G, 246M, 246M/277T/348K/447A, 246M/445R/447A, 260S, 260S/338G, 260S/338G/418G/421S, 277T/347N/348K/445R, 277T/347N/447A, 284D, 284D/332G, 284D/386E, 284D/386E/429G, 310S/338S/418G, 310S/418G/421S, 332G, 332G/386E, 332G/429G, 338G, 338G/418G, 347N/348K/445R, 347N/348K/447A, 348K, 348K/445R/447A, 348K/447A, 386E, 386E/429G, 418G, 429G, and 447A, wherein the positions are numbered with reference to SEQ ID NO: 6. In some further embodiments, the engineered polypeptide comprises at least at least one substitution or one substitution set selected from W79A/S80H, W79A/T93G/Y238R/H386E/A429G, W79A/T93G/H386E, W79A/Y238R, W79A/Y238R/I332G/A429G, W79A/Y238R/A429G, W79D, W79D/S80V, W79D/S80V/I332G/A429G, W79D/T93G, W79D/Y183F/N284D/I332G, S80H, S80H/Y183F/N284D/I332G/C381A/H386E/A429G, S80H/Y188F/E190D/I332G/H386E, S80H/Y238R/H386E/A429G, S80H/I332G/C381A/H386E, S80H/C381A, S80H/H386E, S80H/A429G, S80V, S80V/T237S/I332G, S80V/Y238R/I332G, S80V/N284D, S80V/N284D/H386E, S80V/I332G, S80V/I332G/H386E, S80V/H386E, S80V/H386E/A429G, N82F, N82F/P87G/N113G/P117K/L219S, N82F/P87G/N113G/P117K/R260S/C338G, N82F/P87G/P117K, N82F/P87G/T218G, N82F/P87G/T218G/L219S/C421S, N82F/P87G/T218G/C338S, N82F/P87G/C338S/H418G, N82F/P87L, N82F/P87L/N113G/P117K/T218G, N82F/P87L/N113G/P117K/L219S/H418G, N82F/P87L/N113G/P117K/C338G/H418G, N82F/P87L/P117K, N82F/P87L/L219S/C421S, N82F/P87L/C338S, N82F/N113G/T218G/C338S/H418G, N82F/H418G, N82R/P87G, N82R/P87G/N113G/C421S, N82R/P87G/P117K/T218G/C421S, N82R/P87G/P117K/R260S/C338S/C421S, N82R/P87G/P117K/C338A/H418G, N82R/P87L, N82R/P87L/N113G, N82R/P87L/T218G/L219S/C338S, N82R/P87L/L219S, N82R/P87L/C338G/C421S, N82R/N113G/T218G/C338A, N82R/N113G/L219S/C338S, N82R/N113G/C338S, N82R/T218G/L219S/C421S, N82R/R260S/C338A/C421S, N82R/C338A/H418G/C421S, P87G, P87G/N113G, P87G/N113G/P117K/H418G/C421S, P87G/N113G/R260S, P87G/P117K/L219S/L310S/C338S, P87G/T218G/L219S/C338S, P87G/L219S, P87G/R260S, P87G/L310S, P87G/L310S/C338S, P87G/C338A, P87G/H418G, P87L, P87L/N113G, P87L/N113G/P117K/L219S/C338G/H418G/C421S, P87L/N113G/P117K/R260S/C338S/C421S, P87L/N113G/T218G/L219S/R260S/C338A, P87L/N113G/L219S/C421S, P87L/P117K, P87L/P117K/T218G/L219S, P87L/T218G, P87L/T218G/L219S, P87L/T218G/H418G, P87L/L219S, P87L/L219S/C338G, P87L/L219S/H418G, P87L/C338A, P87L/C421S, T93G/D184R/A429G, T93G/Y238R, T93G/Y238R/N284D, T93G/Y238R/H386E, T93G/Y238R/H386E/A429G, S100P, S100P/E143D/E145S/D146A/L149M/E198D/M242I/C347N/P348K/L445R/C447A, S100P/E143V/E145G/L149M/C347N/C447A, S100P/E145G/D146A/L149M/F250V/C277T/C347N/P348K/C447A, S100P/E145G/L149M/H154L/I246M/C277T, S100P/E145G/L149M/E198D/M242I/I246M/L445R/C447A, S100P/E145G/L149M/E198D/D252G/P348K/L445R, S100P/E145G/L149M/S205T, S100P/E145G/L149M/M242U/246M/C347D/P348K/L445R/C447A, S100P/E145G/L149M/M242I/L445R/C447A, S100P/E145G/E198D/M242I/I246M/P348K, S100P/E145G/E198D/C277T/P348K/L445R, S100P/E145G/E198D/A300V, S100P/E145G/S205T/I246M/C277T/P348K/L445R, S100P/E145G/M242I/I246M/C277T/L445R/C447A, S100P/E145G/P348K, S100P/E145S/D146A/L149M, S100P/E145S/D146A/L149M/H154L/P348K/L445R, S100P/E145S/D146A/L149M/E198D, S100P/E145S/D146A/L149M/E198D/M242I/F250V/D252G/C347N/P348K/C447A, S100P/E145S/D146A/L149M/S205T/C347D/L445R/C447A, S100P/E145S/D146A/L149M/M242I/L445R, S100P/E145S/L149M/E198D/M242I/I246M/C277T/P348K/C447A, S100P/E145S/L149M/E198D/M242I/C277T/C347N/P348K/C447A, S100P/E145S/L149M/M242I/I246M/C277T/C347N/L445R, S100P/E145S/L149M/I246M/D252G/C277T/C347N/L445R/C447A, S100P/E145S/L149M/I246M/C347N/L445R, S100P/E145S/L149M/C277T/P348K/L445R, S100P/E145S/L149M/C347N/L445R/C447A, S100P/E145S/S205T/C277T/P348K, S100P/E145S/I246M/C347N/P348K/L445R, S100P/E145S/C277T/C347N, S100P/D146A/L149M/E198D/M242I/I246M/C277T/C347D/P348K/L445R/C447A, S100P/D146A/E198D/C347D/P348K/C447A, S100P/D146A/E198D/L445R, S100P/D146A/M242I/C277T/C447A, S100P/D146A/F250V/C447A, S100P/D146A/C347D/P348K/L445R/C447A, S100P/L149M/E198D, S100P/L149M/M242U/246M/C277T/C347N/P348K/L445R, S100P/L149M/M242U/246M/C277T/C347N/P348K/C447A, S100P/L149M/M242I/C347N/P348K/C447A, S100P/L149M/I246M/C277T/P348K/L445R, S100P/L149M/I246M/C447A, S100P/L149M/F250V/C347N/C447A, S100P/E198D/S205T/P348K, S100P/E198D/C277T/C347N/P348K, S100P/E198D/C347D/P348K, S100P/E198D/C347D/P348K/L445R, S100P/E198D/C347N/L445R, S100P/E198D/C447A, S100P/M242I/I246M/C277T, S100P/M242I/I246M/C347N, S100P/M242I/C277T/P348K, S100P/M242I/C347D/P348K/L445R, S100P/M242I/C347D/P348K/L445R/C447A, S100P/M242I/P348K/L445R, S100P/I246M/C277T/P348K/L445R, S100P/C277T/C347D/L445R/C447A, S100P/C277T/P348K, S100P/C277T/P348K/C447A, S100P/C347N/P348K/L445R, S100P/C347N/C447A, S100P/L445R/C447A, K110N/C338G/H418G, N113G/T218G/C338S/C421S, N113G/C338A/C421S, N113G/C338G, P117K/L219S, P117K/R260S, F121C/E198D/C347N/L445R/C447A, E145G/D146A/L149M/C277T/C347D, E145G/D146A/L149M/C347D/P348K, E145G/D146A/C277T/C347N, E145G/D146A/C347D/C447A, E145G/L149M/E198D/C347N/C447A, E145G/L149M/S205T/M242I/C347N/P348K/L445R, E145G/L149M/S205T/I246M/F250V/D252G, E145G/L149M/M242I/P348K, E145G/L149M/I246M, E145G/K155I/L445R/C447A, E145G/E198D/S205T/P348K, E145G/F250V/D252G/P348K, E145G/S255R, E145G/C277T/C347N/L445R, E145S/D146A/L149M/H154L/I246M/C277T/C347N/P348K, E145S/D146A/L149M/E198D/M242I/D252G/C277T/C347N, E145S/D146A/L149M/E198D/C277T/L445R/C447A, E145S/D146A/L149M/S205T/P348K/C447A, E145S/D146A/L149M/F250V/C277T/C347D/P348K/C447A, E145S/D146A/E198D/S205T/M242I/C277T/P348K/C447A, E145S/D146A/E198D/L445R/C447A, E145S/D146A/I246M/C277T/C447A, E145S/D146A/I246M/C347N/P348K/C447A, E145S/L149M/E198D/S205T/P348K, E145S/L149M/E198D/M242I/I246M/C277T/C347N/C447A, E145S/L149M/E198D/M242I/I246M/P348K, E145S/L149M/E198D/M242I/C277T/C347D, E145S/L149M/E198D/I246M/C447A, E145S/L149M/S205T/C277T/L445R, E145S/L149M/M242I, E145S/L149M/I246M/C347D/L445R, E145S/L149M/I246M/C447A, E145S/L149M/C347N/P348K/L445R, E145S/L149M/C347N/C447A, E145S/L149M/C447A, E145S/I246M, E145S/I246M/D252G/C347D/C447A, E145S/D252G/P348K/C447A, E145S/C277T/P348K/C447A, E145S/C347D/P348K/L445R/C447A/V491A, E145S/L445R/C447A, D146A/L149M/S151C/I246M/P348K, D146A/L149M/M242I/I246M/L445R, D146A/L149M/I246M/D252G/C447A, D146A/L149M/C277T/P348K/C447A, D146A/E198D/I246M/C277T, D146A/E198D/C277T/P348K/C447A, D146A/M242I, D146A/M242I/I246M/C277T/C347N/P348K/L445R/C447A, D146A/I246M/C277T/P348K/L445R/C447A, D146A/C277T, D146A/C277T/C347N/P348K, D146A/C277T/C347N/L445R/C447A, D146A/C277T/L445R/C447A, D146A/C347D/P348K/C447A, D146A/P348K, D146A/L445R/C447A, L149M, L149M/E198D/C277T/C347N/P348K, L149M/E198D/P348K, L149M/E198D/L445R, L149M/M242I/I246M, L149M/M242I/I246M/P348K/L445R, L149M/C347N/P348K/L445R/C447A, L149M/L445R, D184R, D184R/N284D, D184R/N284D/A429G, D184R/H386E, E198D/M242I/P348K, E198D/I246M, E198D/F250V, E198D/C277T/P348K/C447A, E198D/L445R, S205T, S205T/I246M/C347N, S205T/D252G/C347D/L445R/C447A, S205T/C347D/P348K/L445R/C447A, T218G, T218G/R260S/C338A, L219S, L219S/C338A/H418G, L219S/C421S, Y238R, Y238R/M242L/N284D, Y238R/N284D/H386E/A429G, Y238R/I332G/A429G, Y238R/C381A/A429G, Y238R/H386E, Y238R/A429G, M242I, M242I/C277T/C347D/P348K, M242I/P348K, M242I/L445R/C447A, M242L/N284D/C381A/A429G, I246M, I246M/C277T/P348K/C447A, I246M/L445R/C447A, R260S, R260S/C338G, R260S/C338G/H418G/C421S, C277T/C347N/P348K/L445R, C277T/C347N/C447A, N284D, N284D/I332G, N284D/H386E, N284D/H386E/A429G, L310S/C338S/H418G, L310S/H418G/C421S, I332G, I332G/H386E, I332G/A429G, C338G, C338G/H418G, C347N/P348K/L445R, C347N/P348K/C447A, P348K, P348K/L445R/C447A, P348K/C447A, H386E, H386E/A429G, H418G, A429G, and C447A, wherein the positions are numbered with reference to SEQ ID NO: 6. In some additional embodiments, the engineered polypeptide comprises an amino acid sequence with at least 80% sequence identity to any even-numbered sequence set forth in SEQ ID NO: 416 to SEQ ID NO: 994.

The present invention also provides an engineered polypeptide comprising an amino acid sequence with at least 80%, 85%, 90%, 91%, 92%, 93%, 94%, 95%, 96%, 97%, 98%, 99% sequence identity to a reference sequence of SEQ ID NO: 6, comprising at least one substitution or one substitution set selected from 80/429, 87/113/117/260/338/421, 87/421, 100/145/146/149/198/242/250/252/347/348/447, 100/145/149/198/242/277/347/348/447, 110/338/418, 145/149/205/246/250/252, 145/246/252/347/447, 145/252/348/447, 238/386/429, and 242/284/381/429, wherein the positions are numbered with reference to SEQ ID NO: 6. In some embodiments, the engineered polypeptide comprises at least one substitution or one substitution set selected from 80H/429G, 87L/113G/117K/260S/338S/4215, 87L/421S, 100P/145S/146A/149M/198D/242I/250V/252G/347N/348K/447A, 100P/145S/149M/198D/242I/277T/347N/348K/447A, 110N/338G/418G, 145G/149M/205T/246M/250V/252G, 145S/246M/252G/347D/447A, 145S/252G/348K/447A, 238R/386E/429G, and 242L/284D/381A/429G, wherein the positions are numbered with reference to SEQ ID NO: 6. In some further embodiments, the engineered polypeptide comprises at least one substitution or one substitution set selected from S80H/A429G, P87L/N113G/P117K/R260S/C338S/C421S, P87L/C421S, S100P/E145S/D146A/L149M/E198D/M242I/F250V/D252G/C347N/P348K/C447A, S100P/E145S/L149M/E198D/M242I/C277T/C347N/P348K/C447A, K110N/C338G/H418G, E145G/L149M/S205T/I246M/F250V/D252G, E145S/I246M/D252G/C347D/C447A, E145S/D252G/P348K/C447A, Y238R/H386E/A429G, and M242L/N284D/C381A/A429G, wherein the positions are numbered with reference to SEQ ID NO: 6.

The present invention also provides an engineered polypeptide comprising an amino acid sequence with at least 80%, 85%, 90%, 91%, 92%, 93%, 94%, 95%, 96%, 97%, 98%, 99% sequence identity to a reference sequence of SEQ ID NO: 414, comprising at least one substitution or one substitution set at one or more positions selected from 87/113/145/198/242/246/262/266, 87/117/149/242, 87/117/242/260/266, 87/145/149/198/447, 87/149/242/260/266, 87/149/260/262/266/381, 87/198, 87/242/246/260/262/266/338, 87/284/338/347, 113/117/146/149/260/262, 113/117/266/381, 113/117/447, 113/145/146/149/242/447, 113/242/347, 113/242/381, 117/145/146/262/284, 117/145/149/284/447, 117/149, 117/198, 117/198/262, 117/242/246/260/266/284/381, 117/242/260/262, 117/246/260/262/381, 117/246/284, 117/266, 145/146/149/260/262/338/347, 145/146/242/260/262/338/347/447, 145/149/198/260/266/338/347, 145/149/260/266/338/347/381/447, 146/149/260/262/347, 149/198/246/260/266/421, 198/447, 242, 242/246, 242/246/338/347, 242/260/262/338, 242/381, 246, 260/262/277/284/447, 260/266, 262, 347/381, and 447, wherein the positions are numbered with reference to SEQ ID NO: 414. In some additional embodiments, the engineered polypeptide comprises at least one substitution or one substitution set selected from 87G/113G/145L/198D/242I/246M/262F/266L, 87G/117K/149M/242I, 87G/145L/149M/198D/447A, 87G/149M/242I/260S/266L, 87L/117K/242I/260S/266L, 87L/149M/260S/262F/266L/381A, 87L/198D, 87L/242I/246M/260S/262F/266L/338G, 87L/284D/338G/347N, 113G/117K/146A/149M/260S/262F, 113G/117K/266L/381A, 113G/117K/447A, 113G/145L/146A/149M/242I/447A, 113G/242I/347N, 113G/242I/381A, 117K/145L/146A/262F/284D, 117K/145L/149M/284D/447A, 117K/149M, 117K/198D, 117K/198D/262F, 117K/242I/246M/260S/266L/284D/381A, 117K/242I/260S/262F, 117K/246M/260S/262F/381A, 117K/246M/284D, 117K/266L, 145L/146A/149M/260S/262F/338G/347N, 145L/146A/242I/260S/262F/338G/347N/447A, 145L/149M/198D/260S/266L/338G/347N, 145L/149M/260S/266L/338G/347N/381A/447A, 146A/149M/260S/262F/347N, 149M/198D/246M/260S/266L/421S, 198D/447A, 242I, 242I/246M, 242I/246M/338G/347N, 242I/260S/262F/338G, 242I/381A, 246M, 260S/262F/277T/284D/447A, 260S/266L, 262F, 347N/381A, and 447A, wherein the positions are numbered with reference to SEQ ID NO: 414. In some embodiments, the engineered polypeptide comprises at least one substitution or one substitution set selected from P87G/N113G/E145L/E198D/M242I/I246M/W262F/A266L, P87G/P117K/L149M/M242I, P87G/E145L/L149M/E198D/C447A, P87G/L149M/M242I/R260S/A266L, P87L/P117K/M242I/R260S/A266L, P87L/L149M/R260S/W262F/A266L/C381A, P87L/E198D, P87L/M242I/I246M/R260S/W262F/A266L/C338G, P87L/N284D/C338G/C347N, N113G/P117K/D146A/L149M/R260S/W262F, N113G/P117K/A266L/C381A, N113G/P117K/C447A, N113G/E145L/D146A/L149M/M242I/C447A, N113G/M242I/C347N, N113G/M242I/C381A, P117K/E145L/D146A/W262F/N284D, P117K/E145L/L149M/N284D/C447A, P117K/L149M, P117K/E198D, P117K/E198D/W262F, P117K/M242I/I246M/R260S/A266L/N284D/C381A, P117K/M242I/R260S/W262F, P117K/I246M/R260S/W262F/C381A, P117K/I246M/N284D, P117K/A266L, E145L/D146A/L149M/R260S/W262F/C338G/C347N, E145L/D146A/M242I/R260S/W262F/C338G/C347N/C447A, E145L/L149M/E198D/R260S/A266L/C338G/C347N, E145L/L149M/R260S/A266L/C338G/C347N/C381A/C447A, D146A/L149M/R260S/W262F/C347N, L149M/E198D/I246M/R260S/A266L/C421S, E198D/C447A, M242I, M242I/I246M, M242I/I246M/C338G/C347N, M242I/R260S/W262F/C338G, M242I/C381A, I246M, R260S/W262F/C277T/N284D/C447A, R260S/A266L, W262F, C347N/C381A, and C447A, wherein the positions are numbered with reference to SEQ ID NO: 414. In some further embodiments, the engineered polypeptide comprises an amino acid sequence with at least 80% sequence identity to any even-numbered sequence set forth in SEQ ID NO: 1000 to SEQ ID NO: 1084.

The present invention also provides an engineered polypeptide comprising an amino acid sequence with at least 80%, 85%, 90%, 91%, 92%, 93%, 94%, 95%, 96%, 97%, 98%, 99% sequence identity to a reference sequence of SEQ ID NO: 414, comprising at least one substitution or one substitution set selected from 87/145/149/198/447, 87/149/260/262/266/381, 113/117/146/149/260/262, 117/145/149/284/447, 145/149/198/260/266/338/347, 146/149/260/262/347, and 149/198/246/260/266/421, wherein the positions are numbered with reference to SEQ ID NO: 414. In some embodiments, the engineered polypeptide comprises at least one substitution or one substitution set selected from 87G/145L/149M/198D/447A, 87L/149M/260S/262F/266L/381A, 113G/117K/146A/149M/260S/262F, 117K/145L/149M/284D/447A, 145L/149M/198D/260S/266L/338G/347N, 146A/149M/260S/262F/347N, and 149M/198D/246M/260S/266L/421S, wherein the positions are numbered with reference to SEQ ID NO: 414. In some further embodiments, the engineered polypeptide comprises at least one substitution or one substitution set selected from P87G/E145L/L149M/E198D/C447A, P87L/L149M/R260S/W262F/A266L/C381A, N113G/P117K/D146A/L149M/R260S/W262F, P117K/E145L/L149M/N284D/C447A, E145L/L149M/E198D/R260S/A266L/C338G/C347N, D146A/L149M/R260S/W262F/C347N, and L149M/E198D/I246M/R260S/A266L/C421S, wherein the positions are numbered with reference to SEQ ID NO: 414.

In some embodiments, the engineered monoamine oxidase polypeptide sequence comprises at least 80%, 85%, 86%, 87%, 88%, 89%, 90%, 91%, 92%, 93%, 94%, 95%, 96%, 97%, 98%, 99% sequence identity to SEQ ID NO: 6, wherein said engineered monoamine oxidase polypeptide comprises at least one substitution at one or more positions selected from 429, 149, 198, 246, 260, 266, and 421, and wherein the amino acid positions of said polypeptide sequence are numbered with reference to SEQ ID NO: 6. In some embodiments, the engineered monoamine oxidase polypeptide sequence comprises at least 80%, 85%, 86%, 87%, 88%, 89%, 90%, 91%, 92%, 93%, 94%, 95%, 96%, 97%, 98%, 99% sequence identity to SEQ ID NO: 6, wherein said engineered monoamine oxidase polypeptide comprises at least one substitution selected from 429G, 149M, 198D, 246M, 260S, 266L, and 421S, and wherein the amino acid positions of said polypeptide sequence are numbered with reference to SEQ ID NO: 6. In some embodiments, the engineered monoamine oxidase polypeptide sequence comprises at least 80%, 85%, 86%, 87%, 88%, 89%, 90%, 91%, 92%, 93%, 94%, 95%, 96%, 97%, 98%, 99% sequence identity to SEQ ID NO: 6, wherein said engineered monoamine oxidase polypeptide comprises at least one substitution selected from A429G, L149M, E198D, I246M, R260S, A266L, and C421S, wherein the amino acid positions of said polypeptide sequence are numbered with reference to SEQ ID NO: 6.

The present invention also provides an engineered polynucleotide encoding at least one engineered polypeptide described in the above paragraphs. In some embodiments, the engineered polynucleotide comprises the odd-numbered sequences set forth in SEQ ID NO: 7 to SEQ ID NO: 1083.

The present invention further provides vectors comprising at least one engineered polynucleotide described above. In some embodiments, the vectors further comprise at least one control sequence.

The present invention also provides host cells comprising the vectors provided herein. In some embodiments, the host cell produces at least one engineered polypeptide provided herein.

The present invention further provides methods of producing an engineered monoamine oxidase, comprising the steps of culturing the host cell provided herein under conditions such that the engineered polynucleotide is expressed and the engineered polypeptide is produced. In some embodiments, the methods further comprise the step of recovering the engineered polypeptide.

The present invention further provides methods of using an engineered monoamine oxidase to produce a variety of fused bicyclic proline analogues. In some embodiments, the methods further comprise steps for isolating and recovering the fused bicyclic proline analogues.

### DESCRIPTION OF THE INVENTION

Unless defined otherwise, all technical and scientific terms used herein generally have the same meaning as commonly understood by one of ordinary skill in the art to which this invention pertains. Generally, the nomenclature used herein and the laboratory procedures of cell culture, molecular genetics, microbiology, organic chemistry, analytical chemistry and nucleic acid chemistry described below are those well-known and commonly employed in the art. Such techniques are well-known and described in numerous texts and reference works well known to those of skill in the art. Standard techniques, or modifications thereof, are used for chemical syntheses and chemical analyses. All patents, patent applications, articles and publications mentioned herein, both supra and infra, are hereby expressly incorporated herein by reference.

Although any suitable methods and materials similar or equivalent to those described herein find use in the practice of the present invention, some methods and materials are described herein. It is to be understood that this invention is not limited to the particular methodology, protocols, and reagents described, as these may vary, depending upon the context they are used by those of skill in the art. Accordingly, the terms defined immediately below are more fully described by reference to the invention as a whole.

It is to be understood that both the foregoing general description and the following detailed description are exemplary and explanatory only and are not restrictive of the present invention. The section headings used herein are for organizational purposes only and not to be construed as limiting the subject matter described. Numeric ranges are inclusive of the numbers defining the range. Thus, every numerical range disclosed herein is intended to encompass every narrower numerical range that falls within such broader numerical range, as if such narrower numerical ranges were all expressly written herein. It is also intended that every maximum (or minimum) numerical limitation disclosed herein includes every lower (or higher) numerical limitation, as if such lower (or higher) numerical limitations were expressly written herein.

As used in this specification and the appended claims, the singular forms “a”, “an” and “the” include plural referents unless the context clearly indicates otherwise. Thus, for example, reference to “a polypeptide” includes more than one polypeptide. Similarly, “comprise,” “comprises,” “comprising” “include,” “includes,” and “including” are interchangeable and not intended to be limiting.

It is to be understood that where descriptions of various embodiments use the term “comprising,” those skilled in the art would understand that in some specific instances, an embodiment can be alternatively described using language “consisting essentially of” or “consisting of.” It is to be further understood that where descriptions of various embodiments use the term “optional” or “optionally” the subsequently described event or circumstance may or may not occur, and that the description includes instances where the event or circumstance occurs and instances in which it does not. It is to be understood that both the foregoing general description, and the following detailed description are exemplary and explanatory only and are not restrictive of this disclosure. The section headings used herein are for organizational purposes only and not to be construed as limiting the subject matter described.

### Abbreviations

The abbreviations used for the genetically encoded amino acids are conventional and are as follows:

| Amino Acid | Three-Letter | One-Letter Abbreviation |
|---|---|---|
| Alanine | Ala | A |
| Arginine | Arg | R |
| Asparagine | Asn | N |
| Aspartate | Asp | D |
| Cysteine | Cys | C |
| Glutamate | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

When the three-letter abbreviations are used, unless specifically preceded by an “L” or a “D” or clear from the context in which the abbreviation is used, the amino acid may be in either the L- or D-configuration about α-carbon (C_{α}). For example, whereas “Ala” designates alanine without specifying the configuration about the α-carbon, “D-Ala” and “L-Ala” designate D-alanine and L-alanine, respectively.

When the one-letter abbreviations are used, upper case letters designate amino acids in the L-configuration about the α-carbon and lower case letters designate amino acids in the D-configuration about the α-carbon. For example, “A” designates L-alanine and “a” designates D-alanine. When polypeptide sequences are presented as a string of one-letter or three-letter abbreviations (or mixtures thereof), the sequences are presented in the amino (N) to carboxy (C) direction in accordance with common convention.

The abbreviations used for the genetically encoding nucleosides are conventional and are as follows: adenosine (A); guanosine (G); cytidine (C); thymidine (T); and uridine (U). Unless specifically delineated, the abbreviated nucleotides may be either ribonucleosides or 2′-deoxyribonucleosides. The nucleosides may be specified as being either ribonucleosides or 2′-deoxyribonucleosides on an individual basis or on an aggregate basis. When nucleic acid sequences are presented as a string of one-letter abbreviations, the sequences are presented in the 5′ to 3′ direction in accordance with common convention, and the phosphates are not indicated.

### Definitions

In reference to the present invention, the technical and scientific terms used in the descriptions herein will have the meanings commonly understood by one of ordinary skill in the art, unless specifically defined otherwise. Accordingly, the following terms are intended to have the following meanings.

“EC” number refers to the Enzyme Nomenclature of the Nomenclature Committee of the International Union of Biochemistry and Molecular Biology (NC-IUBMB). The IUBMB biochemical classification is a numerical classification system for enzymes based on the chemical reactions they catalyze.

“ATCC” refers to the American Type Culture Collection whose biorepository collection includes genes and strains.

“NCBI” refers to National Center for Biological Information and the sequence databases provided therein.

“Protein,” “polypeptide,” and “peptide” are used interchangeably herein to denote a polymer of at least two amino acids covalently linked by an amide bond, regardless of length or post-translational modification (e.g., glycosylation, phosphorylation, lipidation, myristilation, ubiquitination, etc.). Included within this definition are D- and L-amino acids, and mixtures of D- and L-amino acids, as well as polymers comprising D- and L-amino acids, and mixtures of D- and L-amino acids.

“Amino acids” are referred to herein by either their commonly known three-letter symbols or by the one-letter symbols recommended by IUPAC-IUB Biochemical Nomenclature Commission. Nucleotides, likewise, may be referred to by their commonly accepted single letter codes.

As used herein, “polynucleotide” and “nucleic acid’ refer to two or more nucleosides that are covalently linked together. The polynucleotide may be wholly comprised of ribonucleotides (i.e., RNA), wholly comprised of 2′ deoxyribonucleotides (i.e., DNA), or comprised of mixtures of ribo- and 2′ deoxyribonucleotides. While the nucleosides will typically be linked together via standard phosphodiester linkages, the polynucleotides may include one or more non-standard linkages. The polynucleotide may be single-stranded or double-stranded, or may include both single-stranded regions and double-stranded regions. Moreover, while a polynucleotide will typically be composed of the naturally occurring encoding nucleobases (i.e., adenine, guanine, uracil, thymine and cytosine), it may include one or more modified and/or synthetic nucleobases, such as, for example, inosine, xanthine, hypoxanthine, etc. In some embodiments, such modified or synthetic nucleobases are nucleobases encoding amino acid sequences.

“Monoamine oxidase” or “MAO,” as used herein, refers to a wild-type or engineered enzyme having monoamine oxidase activity (EC 1.4.3.4), natively catalyzing the oxidation of a primary amine (or sometimes a secondary or tertiary amine) to a corresponding imine. In one embodiment, the monoamine oxidase polypeptide variants catalyze the asymmetric oxidation of 6,6-dimethyl-3-azabicyclo[3.1.0]hexane to (1R,5S)-6,6-dimethyl-3-azabicyclo[3.1.0]hex-2-ene. The definition of monoamine oxidase activity is intended to be non-limiting with regard to substrates and products, and various examples of known substrates and products are presented in the present disclosure. The monoamine oxidases of the present disclosure are flavoproteins, requiring a FAD co-factor. While the monoamine oxidase enzymes of the present disclosure are derived from Aspergillus niger and Aspergillus oryzae, the present invention is not thus limited, and monoamine oxidase enzymes may be derived from any suitable organism or created synthetically. Monoamine oxidases, as used herein, include naturally occurring (wild-type) monoamine oxidases as well as non-naturally occurring engineered polypeptides generated by human manipulation.

“Coding sequence” refers to that portion of a nucleic acid (e.g., a gene) that encodes an amino acid sequence of a protein.

“Naturally-occurring” or “wild-type” refers to the form found in nature. For example, a naturally occurring or wild-type polypeptide or polynucleotide sequence is a sequence present in an organism that can be isolated from a source in nature and which has not been intentionally modified by human manipulation.

As used herein, “recombinant,” “engineered,” and “non-naturally occurring” when used with reference to a cell, nucleic acid, or polypeptide, refer to a material, or a material corresponding to the natural or native form of the material, that has been modified in a manner that would not otherwise exist in nature. In some embodiments, the cell, nucleic acid or polypeptide is identical a naturally occurring cell, nucleic acid or polypeptide, but is produced or derived from synthetic materials and/or by manipulation using recombinant techniques. Non-limiting examples include, among others, recombinant cells expressing genes that are not found within the native (non-recombinant) form of the cell or express native genes that are otherwise expressed at a different level.

“Percentage of sequence identity” and “percentage homology” are used interchangeably herein to refer to comparisons among polynucleotides or polypeptides, and are determined by comparing two optimally aligned sequences over a comparison window, wherein the portion of the polynucleotide or polypeptide sequence in the comparison window may comprise additions or deletions (i.e., gaps) as compared to the reference sequence for optimal alignment of the two sequences. The percentage may be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Alternatively, the percentage may be calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Those of skill in the art appreciate that there are many established algorithms available to align two sequences. Optimal alignment of sequences for comparison can be conducted, e.g., by the local homology algorithm of Smith and Waterman (Smith and Waterman, Adv. Appl. Math., 2:482 [1981]), by the homology alignment algorithm of Needleman and Wunsch (Needleman and Wunsch, J. Mol. Biol., 48:443 [1970]), by the search for similarity method of Pearson and Lipman (Pearson and Lipman, Proc. Natl. Acad. Sci. USA 85:2444 [1988]), by computerized implementations of these algorithms (e.g., GAP, BESTFIT, FASTA, and TFASTA in the GCG Wisconsin Software Package), or by visual inspection, as known in the art. Examples of algorithms that are suitable for determining percent sequence identity and sequence similarity include, but are not limited to the BLAST and BLAST 2.0 algorithms, which are described by Altschul et al. (See, Altschul et al., J. Mol. Biol., 215: 403-410 [1990]; and Altschul et al., Nucl. Acids Res., 3389-3402 [1977], respectively). Software for performing BLAST analyses is publicly available through the National Center for Biotechnology Information website. This algorithm involves first identifying high scoring sequence pairs (HSPs) by identifying short words of length W in the query sequence, which either match or satisfy some positive-valued threshold score T when aligned with a word of the same length in a database sequence. T is referred to as, the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (See, Henikoff and Henikoff, Proc. Natl. Acad. Sci. USA 89:10915 [1989]). Exemplary determination of sequence alignment and % sequence identity can employ the BESTFIT or GAP programs in the GCG Wisconsin Software package (Accelrys, Madison WI), using default parameters provided.

“Reference sequence” refers to a defined sequence used as a basis for a sequence comparison. A reference sequence may be a subset of a larger sequence, for example, a segment of a full-length gene or polypeptide sequence. Generally, a reference sequence is at least 20 nucleotide or amino acid residues in length, at least 25 residues in length, at least 50 residues in length, or the full length of the nucleic acid or polypeptide. Since two polynucleotides or polypeptides may each (1) comprise a sequence (i.e., a portion of the complete sequence) that is similar between the two sequences, and (2) may further comprise a sequence that is divergent between the two sequences, sequence comparisons between two (or more) polynucleotides or polypeptide are typically performed by comparing sequences of the two polynucleotides or polypeptides over a “comparison window” to identify and compare local regions of sequence similarity. In some embodiments, a “reference sequence” can be based on a primary amino acid sequence, where the reference sequence is a sequence that can have one or more changes in the primary sequence. For instance, a “reference sequence based on SEQ ID NO:4 having at the residue corresponding to X14 a valine” or X14V refers to a reference sequence in which the corresponding residue at X14 in SEQ ID NO:4, which is a tyrosine, has been changed to valine.

“Comparison window” refers to a conceptual segment of at least about 20 contiguous nucleotide positions or amino acids residues wherein a sequence may be compared to a reference sequence of at least 20 contiguous nucleotides or amino acids and wherein the portion of the sequence in the comparison window may comprise additions or deletions (i.e., gaps) of 20 percent or less as compared to the reference sequence (which does not comprise additions or deletions) for optimal alignment of the two sequences. The comparison window can be longer than 20 contiguous residues, and includes, optionally 30, 40, 50, 100, or longer windows.

As used herein, “substantial identity” refers to a polynucleotide or polypeptide sequence that has at least 80 percent sequence identity, at least 85 percent identity, at least between 89 to 95 percent sequence identity, or more usually, at least 99 percent sequence identity as compared to a reference sequence over a comparison window of at least 20 residue positions, frequently over a window of at least 30-50 residues, wherein the percentage of sequence identity is calculated by comparing the reference sequence to a sequence that includes deletions or additions which total 20 percent or less of the reference sequence over the window of comparison. In some specific embodiments applied to polypeptides, the term “substantial identity” means that two polypeptide sequences, when optimally aligned, such as by the programs GAP or BESTFIT using default gap weights, share at least 80 percent sequence identity, preferably at least 89 percent sequence identity, at least 95 percent sequence identity or more (e.g., 99 percent sequence identity). In some embodiments, residue positions that are not identical in sequences being compared differ by conservative amino acid substitutions.

“Corresponding to,” “reference to,” and “relative to” when used in the context of the numbering of a given amino acid or polynucleotide sequence refer to the numbering of the residues of a specified reference sequence when the given amino acid or polynucleotide sequence is compared to the reference sequence. In other words, the residue number or residue position of a given polymer is designated with respect to the reference sequence rather than by the actual numerical position of the residue within the given amino acid or polynucleotide sequence. For example, a given amino acid sequence, such as that of an engineered monoamine oxidase, can be aligned to a reference sequence by introducing gaps to optimize residue matches between the two sequences. In these cases, although the gaps are present, the numbering of the residue in the given amino acid or polynucleotide sequence is made with respect to the reference sequence to which it has been aligned.

“Amino acid difference” or “residue difference” refers to a change in the amino acid residue at a position of a polypeptide sequence relative to the amino acid residue at a corresponding position in a reference sequence. The positions of amino acid differences generally are referred to herein as “Xn,” where n refers to the corresponding position in the reference sequence upon which the residue difference is based. For example, a “residue difference at position X25 as compared to SEQ ID NO: 2” refers to a change of the amino acid residue at the polypeptide position corresponding to position 25 of SEQ ID NO: 2. Thus, if the reference polypeptide of SEQ ID NO: 2 has a valine at position 25, then a “residue difference at position X25 as compared to SEQ ID NO: 2” an amino acid substitution of any residue other than valine at the position of the polypeptide corresponding to position 25 of SEQ ID NO: 2. In most instances herein, the specific amino acid residue difference at a position is indicated as “XnY” where “Xn” specified the corresponding position as described above, and “Y” is the single letter identifier of the amino acid found in the engineered polypeptide (i.e., the different residue than in the reference polypeptide). In some embodiments, more than one amino acid can appear in a specified residue position (i.e., the alternative amino acids can be listed in the form XnY/Z, where Y and Z represent alternate amino acid residues). In some instances (e.g., in Tables 4.2, 5.1, 6.1, 7.1, 8.1, and 9.1) the present invention also provides specific amino acid differences denoted by the conventional notation “AnB”, where A is the single letter identifier of the residue in the reference sequence, “n” is the number of the residue position in the reference sequence, and B is the single letter identifier of the residue substitution in the sequence of the engineered polypeptide. Furthermore, in some instances, a polypeptide of the present invention can include one or more amino acid residue differences relative to a reference sequence, which is indicated by a list of the specified positions where changes are made relative to the reference sequence. In some additional embodiments, the present invention provides engineered polypeptide sequences comprising both conservative and non-conservative amino acid substitutions.

As used herein, “conservative amino acid substitution” refers to a substitution of a residue with a different residue having a similar side chain, and thus typically involves substitution of the amino acid in the polypeptide with amino acids within the same or similar defined class of amino acids. By way of example and not limitation, an amino acid with an aliphatic side chain is substituted with another aliphatic amino acid (e.g., alanine, valine, leucine, and isoleucine); an amino acid with an hydroxyl side chain is substituted with another amino acid with a hydroxyl side chain (e.g., serine and threonine); an amino acid having an aromatic side chain is substituted with another amino acid having an aromatic side chain (e.g., phenylalanine, tyrosine, tryptophan, and histidine); an amino acid with a basic side chain is substituted with another amino acid with a basic side chain (e.g., lysine and arginine); an amino acid with an acidic side chain is substituted with another amino acid with an acidic side chain (e.g., aspartic acid or glutamic acid); and/or a hydrophobic or hydrophilic amino acid is replaced with another hydrophobic or hydrophilic amino acid, respectively. Exemplary conservative substitutions are provided in Table 1 below.

TABLE 1
Conservative Amino Acid Substitution Examples

| Residue | Possible Conservative Substitutions |
|---|---|
| A, L, V, I | Other aliphatic (A, L, V, I) |
| | Other non-polar (A, L, V, I, G, M) |
| G, M | Other non-polar (A, L, V, I, G, M) |
| D, E | Other acidic (D, E) |
| K, R | Other basic (K, R) |
| N, Q, S, T | Other polar |
| H, Y, W, F | Other aromatic (H, Y, W, F) |
| C, P | None |

“Non-conservative substitution” refers to substitution of an amino acid in the polypeptide with an amino acid with significantly differing side chain properties. Non-conservative substitutions may use amino acids between, rather than within, the defined groups and affects (a) the structure of the peptide backbone in the area of the substitution (e.g., proline for glycine), (b) the charge or hydrophobicity, or (c) the bulk of the side chain. By way of example and not limitation, an exemplary non-conservative substitution can be an acidic amino acid substituted with a basic or aliphatic amino acid; an aromatic amino acid substituted with a small amino acid; and a hydrophilic amino acid substituted with a hydrophobic amino acid.

“Deletion” refers to modification to the polypeptide by removal of one or more amino acids from the reference polypeptide. Deletions can comprise removal of 1 or more amino acids, 2 or more amino acids, 5 or more amino acids, 10 or more amino acids, 15 or more amino acids, or 20 or more amino acids, up to 10% of the total number of amino acids, or up to 20% of the total number of amino acids making up the reference enzyme while retaining enzymatic activity and/or retaining the improved properties of an engineered monoamine oxidase enzyme. Deletions can be directed to the internal portions and/or terminal portions of the polypeptide. In various embodiments, the deletion can comprise a continuous segment or can be discontinuous.

“Insertion” refers to modification to the polypeptide by addition of one or more amino acids from the reference polypeptide. In some embodiments, the improved engineered monoamine oxidase enzymes comprise insertions of one or more amino acids to the naturally occurring monoamine oxidase polypeptide as well as insertions of one or more amino acids to other improved monoamine oxidase polypeptides. Insertions can be in the internal portions of the polypeptide, or to the carboxy or amino terminus. Insertions as used herein include fusion proteins as is known in the art. The insertion can be a contiguous segment of amino acids or separated by one or more of the amino acids in the naturally occurring polypeptide.

“Fragment” as used herein refers to a polypeptide that has an amino-terminal and/or carboxy-terminal deletion, but where the remaining amino acid sequence is identical to the corresponding positions in the sequence. Fragments can be at least 14 amino acids long, at least 20 amino acids long, at least 50 amino acids long or longer, and up to 70%, 80%, 90%, 95%, 98%, and 99% of the full-length monoamine oxidase polypeptide, for example the polypeptide of SEQ ID NO: 6 or an engineered monoamine oxidase provided in the even-numbered sequences of SEQ ID NOs: 6-1084.

“Isolated polypeptide” refers to a polypeptide which is substantially separated from other contaminants that naturally accompany it, e.g., protein, lipids, and polynucleotides. The term embraces polypeptides which have been removed or purified from their naturally-occurring environment or expression system (e.g., host cell or in vitro synthesis). The engineered monoamine oxidase enzymes may be present within a cell, present in the cellular medium, or prepared in various forms, such as lysates or isolated preparations. As such, in some embodiments, the engineered monoamine oxidase enzyme can be an isolated polypeptide.

“Substantially pure polypeptide” refers to a composition in which the polypeptide species is the predominant species present (i.e., on a molar or weight basis it is more abundant than any other individual macromolecular species in the composition), and is generally a substantially purified composition when the object species comprises at least about 50 percent of the macromolecular species present by mole or % weight. Generally, a substantially pure monoamine oxidase composition will comprise about 60% or more, about 70% or more, about 80% or more, about 90% or more, about 95% or more, and about 98% or more of all macromolecular species by mole or % weight present in the composition. In some embodiments, the object species is purified to essential homogeneity (i.e., contaminant species cannot be detected in the composition by conventional detection methods) wherein the composition consists essentially of a single macromolecular species. Solvent species, small molecules (<500 Daltons), and elemental ion species are not considered macromolecular species. In some embodiments, the isolated engineered monoamine oxidase polypeptide is a substantially pure polypeptide composition.

“Stereoselective” refers to a preference for formation of one stereoisomer over another in a chemical or enzymatic reaction. Stereoselectivity can be partial, where the formation of one stereoisomer is favored over the other, or it may be complete where only one stereoisomer is formed. When the stereoisomers are enantiomers, the stereoselectivity is referred to as enantioselectivity, the fraction (typically reported as a percentage) of one enantiomer in the sum of both. It is commonly alternatively reported in the art (typically as a percentage) as the enantiomeric excess (e.e.) calculated therefrom according to the formula [major enantiomer−minor enantiomer]/[major enantiomer+minor enantiomer]. Where the stereoisomers are diastereoisomers, the stereoselectivity is referred to as diastereoselectivity, the fraction (typically reported as a percentage) of one diastereomer in a mixture of two diastereomers, commonly alternatively reported as the diastereomeric excess (d.e.). Enantiomeric excess and diastereomeric excess are types of stereomeric excess.

“Highly stereoselective” refers to a chemical or enzymatic reaction that is capable of converting a substrate or substrates (e.g., substrate compound 6,6-dimethyl-3-azabicyclo[3.1.0]hexane), to the corresponding imine product (e.g., to (1R,5S)-6,6-dimethyl-3-azabicyclo[3.1.0]hex-2-ene), with at least about 85% stereomeric excess.

As used herein, “improved enzyme property” refers to at least one improved property of an enzyme. In some embodiments, the present invention provides engineered monoamine oxidase polypeptides that exhibit an improvement in any enzyme property as compared to a reference monoamine oxidase polypeptide or another engineered monoamine oxidase. For the engineered monoamine oxidase polypeptides described herein, the comparison is generally made to the parent enzyme from which the monoamine oxidase is derived, although in some embodiments, the reference enzyme can be another improved engineered monoamine oxidase. Thus, the level of “improvement” can be determined and compared between various monoamine oxidase polypeptides, including wild-type, as well as engineered monoamine oxidases. Improved properties include, but are not limited, to such properties as enzymatic activity (which can be expressed in terms of percent conversion of the substrate), thermo stability, solvent stability, pH activity profile, cofactor requirements, refractoriness to inhibitors (e.g., substrate or product inhibition), stereospecificity, and/or stereoselectivity (including enantioselectivity).

“Increased enzymatic activity” refers to an improved property of the engineered monoamine oxidase polypeptides, which can be represented by an increase in specific activity (e.g., product produced/time/weight protein) or an increase in percent conversion of the substrate to the product (e.g., percent conversion of starting amount of substrate to product in a specified time period using a specified amount of monoamine oxidase) as compared to the reference monoamine oxidase enzyme. Exemplary methods to determine enzyme activity are provided in the Examples. Any property relating to enzyme activity may be affected, including the classical enzyme properties of Kₘ, Vₘₐₓ or k_{cat}, changes of which can lead to increased enzymatic activity. Improvements in enzyme activity can be from about 1.2 times the enzymatic activity of the corresponding parent enzyme, to as much as 2 times, 5 times, 10 times, 20 times, 25 times, 50 times or more enzymatic activity than the naturally occurring or another engineered monoamine oxidase from which the monoamine oxidase polypeptides were derived. Monoamine oxidase activity can be measured by any one of standard assays, such as by monitoring changes in properties of substrates, cofactors, or products. In some embodiments, the amount of products generated can be measured by Liquid Chromatography-Mass Spectrometry (LC-MS). Comparisons of enzyme activities are made using a defined preparation of enzyme, a defined assay under a set condition, and one or more defined substrates, as further described in detail herein. Generally, when lysates are compared, the numbers of cells and the amount of protein assayed are determined as well as use of identical expression systems and identical host cells to minimize variations in amount of enzyme produced by the host cells and present in the lysates.

“Conversion” refers to the enzymatic conversion of the substrate(s) to the corresponding product(s). “Percent conversion” refers to the percent of the substrate that is converted to the product within a period of time under specified conditions. Thus, the “enzymatic activity” or “activity” of a monoamine oxidase polypeptide can be expressed as “percent conversion” of the substrate to the product.

“Thermostable” refers to a monoamine oxidase polypeptide that maintains similar activity (more than 60% to 80% for example) after exposure to elevated temperatures (e.g., 40-80° C.) for a period of time (e.g., 0.5-24 hrs) compared to the parent enzyme exposed to the same elevated temperature.

“Solvent stable” refers to a monoamine oxidase polypeptide that maintains similar activity (more than e.g., 60% to 80%) after exposure to varying concentrations (e.g., 5-99%) of solvent (ethanol, isopropyl alcohol, dimethylsulfoxide (DMSO), tetrahydrofuran, 2-methyltetrahydrofuran, acetone, toluene, butyl acetate, methyl tert-butyl ether, etc.) for a period of time (e.g., 0.5-24 hrs) compared to the parent enzyme exposed to the same concentration of the same solvent.

“Thermo- and solvent stable” refers to a monoamine oxidase polypeptide that is both thermostable and solvent stable.

The term “stringent hybridization conditions” is used herein to refer to conditions under which nucleic acid hybrids are stable. As known to those of skill in the art, the stability of hybrids is reflected in the melting temperature (Tm) of the hybrids. In general, the stability of a hybrid is a function of ion strength, temperature, G/C content, and the presence of chaotropic agents. The Tm values for polynucleotides can be calculated using known methods for predicting melting temperatures (See e.g., Baldino et al., Meth. Enzymol., 168:761-777 [1989]; Bolton et al., Proc. Natl. Acad. Sci. USA 48:1390 [1962]; Bresslauer et al., Proc. Natl. Acad. Sci. USA 83:8893-8897 [1986]; Freier et al., Proc. Natl. Acad. Sci. USA 83:9373-9377 [1986]; Kierzek et al., Biochem., 25:7840-7846 [1986]; Rychlik et al., 1990, Nucl. Acids Res., 18:6409-6412 [1990] (erratum, Nucl. Acids Res., 19:698 [1991]); Sambrook et al., supra); Suggs et al., 1981, in Developmental Biology Using Purified Genes, Brown et al. [eds.], pp. 683-693, Academic Press, Cambridge, MA [1981]; and Wetmur, Crit. Rev. Biochem. Mol. Biol., 26:227-259 [1991]). In some embodiments, the polynucleotide encodes the polypeptide disclosed herein and hybridizes under defined conditions, such as moderately stringent or highly stringent conditions, to the complement of a sequence encoding an engineered monoamine oxidase enzyme of the present invention.

“Hybridization stringency” relates to hybridization conditions, such as washing conditions, in the hybridization of nucleic acids. Generally, hybridization reactions are performed under conditions of lower stringency, followed by washes of varying but higher stringency. The term “moderately stringent hybridization” refers to conditions that permit target-DNA to bind a complementary nucleic acid that has about 60% identity, preferably about 75% identity, about 85% identity to the target DNA, with greater than about 90% identity to target-polynucleotide. Exemplary moderately stringent conditions are conditions equivalent to hybridization in 50% formamide, 5×Denhart's solution, 5×SSPE, 0.2% SDS at 42° C., followed by washing in 0.2×SSPE, 0.2% SDS, at 42° C. “High stringency hybridization” refers generally to conditions that are about 10° C. or less from the thermal melting temperature Tm as determined under the solution condition for a defined polynucleotide sequence. In some embodiments, a high stringency condition refers to conditions that permit hybridization of only those nucleic acid sequences that form stable hybrids in 0.018 M NaCl at 65° C. (i.e., if a hybrid is not stable in 0.018 M NaCl at 65° C., it will not be stable under high stringency conditions, as contemplated herein). High stringency conditions can be provided, for example, by hybridization in conditions equivalent to 50% formamide, 5×Denhart's solution, 5×SSPE, 0.2% SDS at 42° C., followed by washing in 0.1×SSPE, and 0.1% SDS at 65° C. Another high stringency condition is hybridizing in conditions equivalent to hybridizing in 5×SSC containing 0.1% (w:v) SDS at 65° C. and washing in 0.1×SSC containing 0.1% SDS at 65° C. Other high stringency hybridization conditions, as well as moderately stringent conditions, are described in the references cited above.

“Heterologous” polynucleotide refers to any polynucleotide that is introduced into a host cell by laboratory techniques, and includes polynucleotides that are removed from a host cell, subjected to laboratory manipulation, and then reintroduced into a host cell.

“Codon optimized” refers to changes in the codons of the polynucleotide encoding a protein to those preferentially used in a particular organism such that the encoded protein is efficiently expressed in the organism of interest. Although the genetic code is degenerate in that most amino acids are represented by several codons, called “synonyms” or “synonymous” codons, it is well known that codon usage by particular organisms is nonrandom and biased towards particular codon triplets. This codon usage bias may be higher in reference to a given gene, genes of common function or ancestral origin, highly expressed proteins versus low copy number proteins, and the aggregate protein coding regions of an organism's genome. In some embodiments, the polynucleotides encoding the monoamine oxidase enzymes may be codon optimized for optimal production from the host organism selected for expression.

As used herein, “preferred, optimal, high codon usage bias codons” refers interchangeably to codons that are used at higher frequency in the protein coding regions than other codons that code for the same amino acid. The preferred codons may be determined in relation to codon usage in a single gene, a set of genes of common function or origin, highly expressed genes, the codon frequency in the aggregate protein coding regions of the whole organism, codon frequency in the aggregate protein coding regions of related organisms, or combinations thereof. Codons whose frequency increases with the level of gene expression are typically optimal codons for expression. A variety of methods are known for determining the codon frequency (e.g., codon usage, relative synonymous codon usage) and codon preference in specific organisms, including multivariate analysis, for example, using cluster analysis or correspondence analysis, and the effective number of codons used in a gene (See e.g., GCG CodonPreference, Genetics Computer Group Wisconsin Package; CodonW, Peden, University of Nottingham; McInerney, Bioinform., 14:372-73 [1998]; Stenico et al., Nucl. Acids Res., 222437-46 [1994]; Wright, Gene 87:23-29 [1990]). Codon usage tables are available for many different organisms (See e.g., Wada et al., Nucl. Acids Res., 20:2111-2118 [1992]; Nakamura et al., Nucl. Acids Res., 28:292 [2000]; Duret, et al., supra; Henaut and Danchin, in Escherichia coli and Salmonella, Neidhardt, et al. (eds.), ASM Press, Washington D.C., p. 2047-2066 [1996]). The data source for obtaining codon usage may rely on any available nucleotide sequence capable of coding for a protein. These data sets include nucleic acid sequences actually known to encode expressed proteins (e.g., complete protein coding sequences-CDS), expressed sequence tags (ESTS), or predicted coding regions of genomic sequences (See e.g., Mount, Bioinformatics: Sequence and Genome Analysis, Chapter 8, Cold Spring Harbor Laboratory Press, Cold Spring Harbor, N.Y. [2001]; Uberbacher, Meth. Enzymol., 266:259-281 [1996]; and Tiwari et al., Comput. Appl. Biosci., 13:263-270 [1997]).

“Control sequence” is defined herein to include all components, which are necessary or advantageous for the expression of a polynucleotide and/or polypeptide of the present invention. Each control sequence may be native or foreign to the nucleic acid sequence encoding the polypeptide. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the nucleic acid sequence encoding a polypeptide.

“Operably linked” is defined herein as a configuration in which a control sequence is appropriately placed (i.e., in a functional relationship) at a position relative to a polynucleotide of interest such that the control sequence directs or regulates the expression of the polynucleotide and/or polypeptide of interest.

“Promoter sequence” refers to a nucleic acid sequence that is recognized by a host cell for expression of a polynucleotide of interest, such as a coding sequence. The promoter sequence contains transcriptional control sequences, which mediate the expression of a polynucleotide of interest. The promoter may be any nucleic acid sequence which shows transcriptional activity in the host cell of choice including mutant, truncated, and hybrid promoters, and may be obtained from genes encoding extracellular or intracellular polypeptides either homologous or heterologous to the host cell.

“Suitable reaction conditions” refer to those conditions in the biocatalytic reaction solution (e.g., ranges of enzyme loading, substrate loading, cofactor loading, temperature, pH, buffers, co-solvents, etc.) under which monoamine oxidase polypeptide of the present invention is capable of converting one or more substrate compounds to a product compound (e.g., conversion of compounds of Formula I to compounds of Formula II, as shown in Scheme 1). Exemplary “suitable reaction conditions” are provided in the present invention and illustrated by the Examples.

“Composition” refers to a mixture or combination of one or more substances, wherein each substance or component of the composition retains its individual properties. As used herein, a biocatalytic composition refers to a combination of one or more substances useful for biocatalysis.

“Loading”, such as in “compound loading” or “enzyme loading” or “cofactor loading” refers to the concentration or amount of a component in a reaction mixture at the start of the reaction.

“Substrate” in the context of a biocatalyst mediated process refers to the compound or molecule acted on by the biocatalyst. For example, 6,6-dimethyl-3-azabicyclo[3.1.0]hexane is a substrate for a monoamine oxidase.

“Product” in the context of a biocatalyst mediated process refers to the compound or molecule resulting from the action of the biocatalyst. For example, (1R,5S)-6,6-dimethyl-3-azabicyclo[3.1.0]hex-2-ene is a product of the monoamine oxidase-mediated conversion of 6,6-dimethyl-3-azabicyclo[3.1.0]hexane).

“Alkyl” refers to saturated hydrocarbon groups of from 1 to 18 carbon atoms inclusively, either straight chained or branched, more preferably from 1 to 8 carbon atoms inclusively, and most preferably 1 to 6 carbon atoms inclusively. An alkyl with a specified number of carbon atoms is denoted in parenthesis (e.g., (C₁-C₆)alkyl refers to an alkyl of 1 to 6 carbon atoms).

“Alkenyl” refers to hydrocarbon groups of from 2 to 12 carbon atoms inclusively, either straight or branched containing at least one double bond but optionally containing more than one double bond.

“Alkynyl” refers to hydrocarbon groups of from 2 to 12 carbon atoms inclusively, either straight or branched containing at least one triple bond but optionally containing more than one triple bond, and

“Heteroalkyl, “heteroalkenyl,” and heteroalkynyl,” refer respectively, to alkyl, alkenyl and alkynyl as defined herein in which one or more of the carbon atoms are each independently replaced with the same or different heteroatoms or heteroatomic groups. Heteroatoms and/or heteroatomic groups which can replace the carbon atoms include, but are not limited to, —O—, —S—, —S—O—, —NRα—, —PH—, —S(O)—, —S(O)2-, —S(O) NRα-, —S(O)2NRα-, and the like, including combinations thereof, where each Ra is independently selected from hydrogen, alkyl, cycloalkyl, heterocycloalkyl, aryl, and heteroaryl.

“Aryl” refers to an unsaturated aromatic carbocyclic group of from 6 to 12 carbon atoms inclusively having a single ring (e.g., phenyl) or multiple condensed rings (e.g., naphthyl or anthryl). Exemplary aryls include phenyl, pyridyl, naphthyl and the like.

“Arylalkyl” refers to an alkyl substituted with an aryl (i.e., aryl-alkyl-groups), preferably having from 1 to 6 carbon atoms inclusively in the alkyl moiety and from 6 to 12 carbon atoms inclusively in the aryl moiety. Such arylalkyl groups are exemplified by benzyl, phenethyl and the like.

“Cycloalkyl” refers to cyclic alkyl groups of from 3 to 12 carbon atoms inclusively having a single cyclic ring or multiple condensed rings which can be optionally substituted with from 1 to 3 alkyl groups. Exemplary cycloalkyl groups include, but are not limited to, single ring structures such as cyclopropyl, cyclobutyl, cyclopentyl, cyclooctyl, 1-methylcyclopropyl, 2-methylcyclopentyl, 2-methylcyclooctyl, and the like, or multiple ring structures, including bridged ring systems, such as adamantyl, and the like.

“Cycloalkylalkyl” refers to an alkyl substituted with a cycloalkyl (i.e., cycloalkyl-alkyl-groups), preferably having from 1 to 6 carbon atoms inclusively in the alkyl moiety and from 3 to 12 carbon atoms inclusively in the cycloalkyl moiety. Such cycloalkylalkyl groups are exemplified by cyclopropylmethyl, cyclohexylethyl and the like.

“Amino” refers to the group —NH2. Substituted amino refers to the group —NHRδ, NRδRδ, and NRδRδRδ, where each Rδ is independently selected from substituted or unsubstituted alkyl, cycloalkyl, cycloheteroalkyl, alkoxy, aryl, heteroaryl, heteroarylalkyl, acyl, alkoxycarbonyl, sulfanyl, sulfinyl, sulfonyl, and the like. Typical amino groups include, but are limited to, dimethylamino, diethylamino, trimethylammonium, triethylammonium, methylysulfonylamino, furanyl-oxy-sulfamino, and the like.

“Aminoalkyl” refers to an alkyl group in which one or more of the hydrogen atoms are replaced with one or more amino groups, including substituted amino groups.

“Aminocarbonyl” refers to —C(O)NH₂. Substituted aminocarbonyl refers to —C(O)NRδRδ, where the amino group NRδRδ is as defined herein.

“Oxy” refers to a divalent group —O—, which may have various substituents to form different oxy groups, including ethers and esters.

“Alkoxy” or “alkyloxy” are used interchangeably herein to refer to the group —ORβ wherein Rβ is an alkyl group, including optionally substituted alkyl groups.

“Carboxy” refers to —COOH.

“Carbonyl” refers to —C(O)—, which may have a variety of substituents to form different carbonyl groups including acids, acid halides, aldehydes, amides, esters, and ketones.

“Halogen” or “halo” refers to fluoro, chloro, bromo and iodo.

“Haloalkyl” refers to an alkyl group in which one or more of the hydrogen atoms are replaced with a halogen. Thus, the term “haloalkyl” is meant to include monohaloalkyls, dihaloalkyls, trihaloalkyls, etc. up to perhaloalkyls. For example, the expression “(C₁-C₂) haloalkyl” includes 1-fluoromethyl, difluoromethyl, trifluoromethyl, 1-fluoroethyl, 1,1-difluoroethyl, 1,2-difluoroethyl, 1,1,1 trifluoroethyl, perfluoroethyl, etc.

“Hydroxy” refers to —OH.

“Hydroxyalkyl” refers to an alkyl group in which in which one or more of the hydrogen atoms are replaced with one or more hydroxy groups.

“Thiol” or “sulfanyl” refers to —SH. Substituted thiol or sulfanyl refers to —S—R^{η}, where R^{η} is an alkyl, aryl or other suitable substituent.

“Alkylthio” refers to —SR^{ζ}, where R^{ζ} is an alkyl, which can be optionally substituted. Typical alkylthio group include, but are not limited to, methylthio, ethylthio, n-propylthio, and the like.

“Alkylthioalkyl” refers to an alkyl substituted with an alkylthio group, —SR^{ζ}, where R^{ζ} is an alkyl, which can be optionally substituted.

“Sulfonyl” refers to —SO₂—. Substituted sulfonyl refers to —SO₂—R^{η}, where R^{η} is an alkyl, aryl or other suitable substituent.

“Alkylsulfonyl” refers to —SO₂—R^{ζ}, where R^{ζ} is an alkyl, which can be optionally substituted.

Typical alkylsulfonyl groups include, but are not limited to, methylsulfonyl, ethylsulfonyl, n-propylsulfonyl, and the like.

“Alkylsulfonylalkyl” refers to an alkyl substituted with an alkylsulfonyl group, —SO₂—R^{ζ}, where R^{ζ} is an alkyl, which can be optionally substituted.

“Heteroaryl” refers to an aromatic heterocyclic group of from 1 to 10 carbon atoms inclusively and 1 to 4 heteroatoms inclusively selected from oxygen, nitrogen and sulfur within the ring. Such heteroaryl groups can have a single ring (e.g., pyridyl or furyl) or multiple condensed rings (e.g., indolizinyl or benzothienyl).

“Heteroarylalkyl” refers to an alkyl substituted with a heteroaryl (i.e., heteroaryl-alkyl-groups), preferably having from 1 to 6 carbon atoms inclusively in the alkyl moiety and from 5 to 12 ring atoms inclusively in the heteroaryl moiety. Such heteroarylalkyl groups are exemplified by pyridylmethyl and the like.

“Heterocycle”, “heterocyclic” and interchangeably “heterocycloalkyl” refer to a saturated or unsaturated group having a single ring or multiple condensed rings, from 2 to 10 carbon ring atoms inclusively and from 1 to 4 hetero ring atoms inclusively selected from nitrogen, sulfur or oxygen within the ring. Such heterocyclic groups can have a single ring (e.g., piperidinyl or tetrahydrofuryl) or multiple condensed rings (e.g., indolinyl, dihydrobenzofuran or quinuclidinyl). Examples of heterocycles include, but are not limited to, furan, thiophene, thiazole, oxazole, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, pyrrolidine, indoline and the like.

“Membered ring” is meant to embrace any cyclic structure. The number preceding the term “membered” denotes the number of skeletal atoms that constitute the ring. Thus, for example, cyclohexyl, pyridine, pyran and thiopyran are 6-membered rings and cyclopentyl, pyrrole, furan, and thiophene are 5-membered rings.

“Fused bicyclic ring” as used herein refers to both unsubstituted and substituted carbocyclic and/or heterocyclic ring moieties having 3 to 8 atoms in each ring, the rings having 2 common atoms.

“Optionally substituted” as used herein with respect to the foregoing chemical groups means that positions of the chemical group occupied by hydrogen can be substituted with another atom (unless otherwise specified) exemplified by, but not limited to carbon, oxygen, nitrogen, or sulfur, or a chemical group, exemplified by, but not limited to, hydroxy, oxo, nitro, methoxy, ethoxy, alkoxy, substituted alkoxy, trifluoromethoxy, haloalkoxy, fluoro, chloro, bromo, iodo, halo, methyl, ethyl, propyl, butyl, alkyl, alkenyl, alkynyl, substituted alkyl, trifluoromethyl, haloalkyl, hydroxyalkyl, alkoxyalkyl, thio, alkylthio, acyl, carboxy, alkoxycarbonyl, carboxamido, substituted carboxamido, alkylsulfonyl, alkylsulfinyl, alkylsulfonylamino, sulfonamido, substituted sulfonamido, cyano, amino, substituted amino, alkylamino, dialkylamino, aminoalkyl, acylamino, amidino, amidoximo, hydroxamoyl, phenyl, aryl, substituted aryl, aryloxy, arylalkyl, arylalkenyl, arylalkynyl, pyridyl, imidazolyl, heteroaryl, substituted heteroaryl, heteroaryloxy, heteroarylalkyl, heteroarylalkenyl, heteroarylalkynyl, cyclopropyl, cyclobutyl, cyclopentyl, cyclohexyl, cycloalkyl, cycloalkenyl, cycloalkylalkyl, substituted cycloalkyl, cycloalkyloxy, pyrrolidinyl, piperidinyl, morpholino, heterocycle, (heterocycle)oxy, and (heterocycle)alkyl; where preferred heteroatoms are oxygen, nitrogen, and sulfur. Additionally, where open valences exist on these substitute chemical groups they can be further substituted with alkyl, cycloalkyl, aryl, heteroaryl, and/or heterocycle groups, that where these open valences exist on carbon they can be further substituted by halogen and by oxygen-, nitrogen-, or sulfur-bonded substituents, and where multiple such open valences exist, these groups can be joined to form a ring, either by direct formation of a bond or by formation of bonds to a new heteroatom, preferably oxygen, nitrogen, or sulfur. It is further contemplated that the above substitutions can be made provided that replacing the hydrogen with the substituent does not introduce unacceptable instability to the molecules of the present invention, and is otherwise chemically reasonable. One of ordinary skill in the art would understand that with respect to any chemical group described as optionally substituted, only sterically practical and/or synthetically feasible chemical groups are meant to be included. “Optionally substituted” as used herein refers to all subsequent modifiers in a term or series of chemical groups. For example, in the term “optionally substituted arylalkyl,” the “alkyl” portion and the “aryl” portion of the molecule may or may not be substituted, and for the series “optionally substituted alkyl, cycloalkyl, aryl and heteroaryl,” the alkyl, cycloalkyl, aryl, and heteroaryl groups, independently of the others, may or may not be substituted.

“Reaction” as used herein refers to a process in which one or more substances or compounds or substrates is converted into one or more different substances, compounds, or processes.

### Oxidation of Bicyclic Proline Analogue Amines to Imines

The present invention provides novel biocatalysts and associated methods of use for the oxidative desymmetrization of fused bicyclic proline analogues to produce APIs and intermediates. The novel biocatalysts of the present disclosure are engineered monoamine oxidase enzymes with improved solubility, thermostability, and activity on fused bicyclic proline compounds, as compared to a reference monoamine oxidase. In particular, the engineered monoamine oxidase enzymes of the present disclosure require reduced enzyme loading (wt/wt) for the manufacturing of APIs and intermediates, as compared to a reference monoamine oxidase enzyme.

The monoamine oxidase biocatalysts of the present disclosure are engineered polypeptide variants of a previously engineered monoamine oxidase (SEQ ID NO: 6) derived from the monoamine oxidase of Aspergillus niger (SEQ ID NO: 2) and the monoamine oxidase of Aspergillus oryzae (SEQ ID NO: 4) WO2010/008828A2. These engineered polypeptides are capable of catalyzing the asymmetric oxidation of various fused bicyclic proline analogue amine compounds to imine compounds. In one embodiment, the monoamine oxidase polypeptide variants catalyze the conversion of 6,6-dimethyl-3-azabicyclo[3.1.0]hexane to (1R,5S)-6,6-dimethyl-3-azabicyclo[3.1.0]hex-2-ene. In other embodiments, the engineered monoamine oxidase enzymes of the present disclosure are used to produce various bicyclic proline analogues.

Therefore, in specific embodiments, the present disclosure provides engineered monoamine oxidase enzymes for preparation of the following fused bicyclic proline compounds, which are useful as intermediates for the synthesis of one or more therapeutic agents:

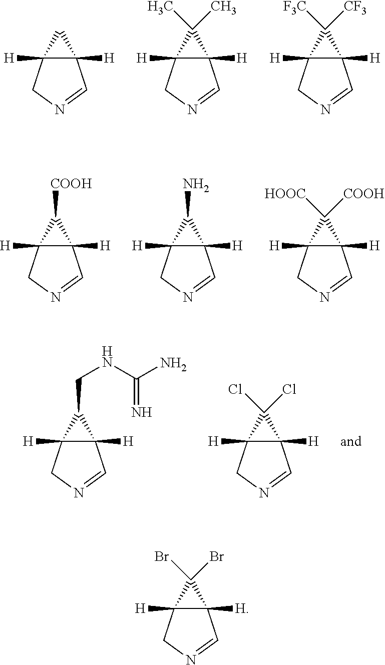

In certain other specific embodiments, the present disclosure provides biocatalytic processes for the synthesis of the following fused bicyclic proline compounds useful as intermediates for the synthesis of one or more therapeutic agents:

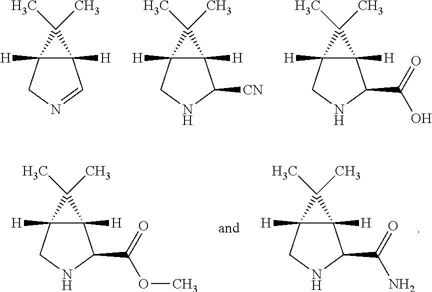

Thus, the present disclosure provides engineered monoamine oxidase enzymes for the synthesis of fused bicyclic proline compounds useful as intermediates for the synthesis of one or more therapeutic agents, as well as biocatalytic processes for the synthesis of these fused bicyclic proline compounds. The engineered monoamine oxidase enzymes of the present disclosure are useful in the biocatalytic processes disclosed below.

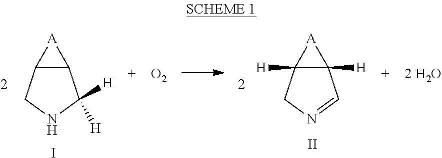

Scheme 1 depicts the reaction catalyzed by the engineered monoamine oxidase enzymes of the disclosure whereby a secondary amine, i.e., a heterobicyclic compound according to structural Formula I is oxidized to the corresponding imine compound of structural Formula II.

In some embodiments, the present disclosure provides a method of preparing a substantially stereomerically pure compound according to structural Formula II:

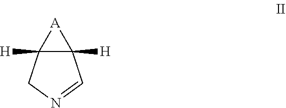

including salts and hydrates thereof, wherein:
   A is O, CR¹R², —C═C—, or —CH₂—CH₂—, wherein R¹ and R² are each independently selected from —H, —COOH, —X, —NH₂, —CH₂NHC(NH)NH₂, —CX₃, —CH₃, —CH₂CH₃, and wherein X is selected from F, Cl, and Br;
   the method comprising contacting an amine compound according to structural Formula I

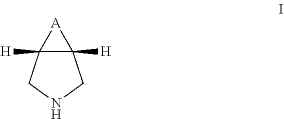

wherein A, is as defined for structural Formula II, with oxygen in the presence of an engineered monoamine oxidase enzyme with a co-factor under conditions in which the engineered monoamine oxidase enzyme oxidizes the amine compound of structural Formula I to a corresponding imine compound of structural Formula II.

In certain embodiments, the oxidized imine compounds of structural Formula II may be isolated from the reaction mixture, purified, and characterized. In other embodiments, described below, the imine compounds of structural Formula II may be converted to another adduct or intermediate and used in a subsequent step without isolation or purification.

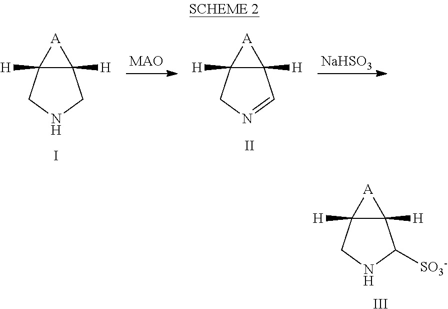

In some embodiments, the present disclosure provides a method of preparing a mixture comprising a substantially enantiomerically pure aminosulfonate compound according to structural Formula III(a) and a substantially enantiomerically pure aminosulfonate compound according to structural Formula III(b):

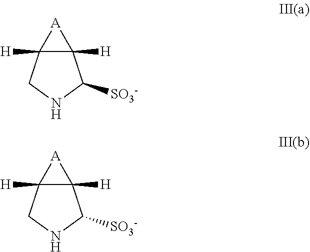

including salts and hydrates thereof, wherein:
   A is O, CR¹R², —C═C—, or —CH₂—CH₂—, wherein R¹ and R² are each independently selected from —H, —COOH, —X, —NH₂, —CH₂NHC(NH)NH₂, —CX₃, —CH₃, —CH₂CH₃, and wherein X is selected from F, Cl, and Br;
   the method comprising contacting an amine compound according to structural Formula I

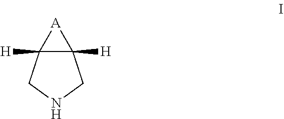

wherein A is as defined for structural Formula III with oxygen in the presence of an engineered monoamine oxidase enzyme associated with a co-factor and with bisulfite under conditions which yield the mixture comprising a substantially enantiomerically aminosulfonate compound according to structural Formula III(a) and a substantially enantiomerically aminosulfonate compound according to structural Formula III(b).

In certain embodiments, the engineered monoamine oxidase of the disclosure may be inhibited by the product imine of structural Formula II. Accordingly, in particular embodiments, the reaction in which a compound of structural Formula I is oxidized to a compound of structural Formula II further comprises an agent that will react with the imine compound of structural Formula II to form an adduct with a reduced or eliminated ability to inhibit an engineered monoamine oxidase of the disclosure, as depicted in Scheme 2.

In one aspect of this embodiment, the agent is added at the beginning of the reaction or is added intermittently or continuously in amounts sufficiently high to prevent accumulation of an inhibitory amount of the imine compound of structural Formula II but low enough to avoid accumulation of an enzyme-inhibitory amount of that agent. In one embodiment, the agent is sodium bisulfite, which may be conveniently supplied as sodium metabisulfite, which hydrates in water to sodium bisulfite. Reaction of bisulfite with the imine compound of structural Formula II provides the sulfite adducts of structural Formula III. In certain embodiments, sodium bisulfite is added continuously to the reaction at a rate whereby this reagent is “instantaneously” consumed and the potentially inhibitory imine product of structural Formula II is “trapped” as the less inhibitory or non-inhibitory sulfite adduct compound of structural Formula III.

Whether the engineered monoamine oxidase is inhibited by the imine product or not, the addition of bisulfite to react the imine product also provides practical process engineering options. Certain imines of the invention are highly volatile and among those, some are malodorous and/or noxious. Their containment as free bases requires closed reactions, without gas flow, or efficient condensation of chemical trapping (e.g. by a bisulfite solution). In situ reaction to their bisulfite adducts (the aminosulfonates), obviates these engineering constraints while also providing the option to conduct the subsequent reaction with cyanide in the same reaction vessel.

Formation of the bisulfite adduct compound structural Formula III can be reversed at elevated pH, whereby the corresponding imine of structural Formula II is regenerated. Accordingly, in certain embodiments, the reaction of Scheme 2 is quenched by addition of base, e.g., 10 N NaOH, to raise the pH to about 13, regenerating the imine of structural Formula II which can be extracted, e.g., with methyl t-butyl ether (“MBTE”) and, in certain embodiments, isolated by distillation to provide an imine of structural Formula II as a colorless oil.

In certain embodiments, the rates of addition the substrate of structural Formula I, the sequestration agent (e.g., sodium bisulfite), and a pH control agent (e.g. NaOH) are monitored and controlled, in part, to minimize or obviate both substrate and product inhibition of the engineered monoamine oxidase used as the biocatalyst for conversion of a compound of structural Formula I to a compound of structural Formula II.

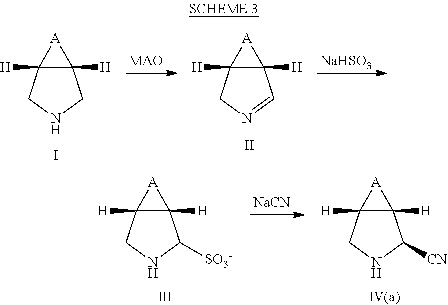

In some embodiments, the present disclosure provides a method of preparing a substantially enantiomerically pure aminonitrile compound according to structural Formula IV(a):

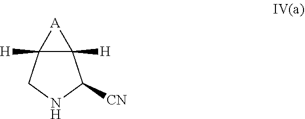

including salts and hydrates thereof, wherein:
   A is O, CR¹R², —C═C—, or —CH₂—CH₂—, wherein R¹ and R² are each independently selected from —H, —COOH, —X, —NH₂, —CH₂NHC(NH)NH₂, —CX₃, —CH₃, —CH₂CH₃, and wherein X is selected from F, Cl, and Br;
      the method comprising contacting an amine compound according to structural Formula I

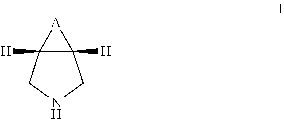

wherein A is as defined for structural Formula IV with oxygen in the presence of an engineered monoamine oxidase enzyme associated with a co-factor and with bisulfite under conditions which yield a mixture comprising a substantially enantiomerically pure aminosulfonate compound according to structural Formula III(a) and a substantially enantiomerically pure aminosulfonate compound according to structural Formula III(b)

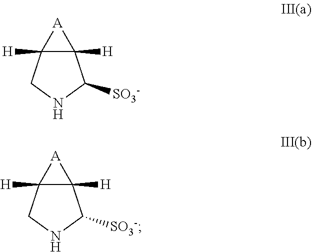

and
contacting the compounds according to structural Formulae III(a) and III(b) with cyanide under conditions which yield the substantially enantiomerically pure aminonitrile compound according to structural Formula IV(a).

In another embodiment, NaCN is added to the reaction of Scheme 2 and the pH allowed to rise to about pH 10, whereby the sulfite adducts of structural Formula III are stereoselectively converted to the trans aminonitrile compounds of structural Formula IV(a), as depicted in Scheme 3. In aspects of this embodiment, about 1 to about 3 equivalents, about 1 to about 2, about 1.05 to about 1.5 equivalents, or about 1.1 to about 1.2 equivalents of NaCN (relative to the imine compound of structural Formula II are added to the reaction to convert the sulfite adducts of structural Formula III to the trans aminonitrile compounds of structural Formula IV(a). Additionally, the reaction of the compound of Formula III produces the cis aminonitrile compounds of structural Formula IV(b).

The trans aminonitrile compounds of structural Formula IV(a) can be extracted from the reaction mixture (1:1 organic solvent: aqueous reaction mixture) using, e.g., 2-methyl tetrahydrofuran, MTBE, or iso-propyl acetate. The trans aminonitrile compound can be recovered from the organic solvent extract, e.g. the organic extract, with optional intermediate further clarifications, can be concentrated under reduced pressure to provide the aminonitrile compounds of structural Formula IV.

In another embodiment, the trans aminonitrile compounds of structural Formula IV(a) prepared e.g. according to Scheme 3 can be contacted with aqueous acid (e.g. HCl or H₂SO₄) to provide amino acids. In yet another embodiment, corresponding t-butyl esters can also be prepared by contacting the amine compounds with an acid (e.g., methane sulfonic acid) and isobutylene or a t-butyl ester (e.g., t-butyl acetate).

In another embodiment, the trans aminonitrile compounds of structural Formula IV(a) prepared e.g., according to Scheme 3 can be contacted with HCl and methanol in a Pinner reaction to provide methyl esters.

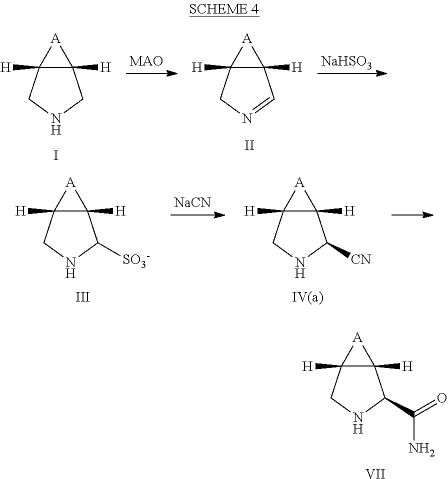

Scheme 4 depicts an overall process for the preparation of compounds according to structural Formula VII from the secondary amines of structural Formula I.

In some embodiments, the present disclosure provides a method of preparing a substantially stereomerically pure amino amide compound according to structural Formula VII:

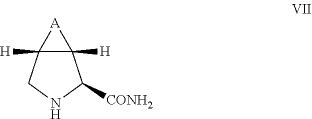

including salts thereof, wherein:
   A is O, CR¹R², —C═C—, or —CH₂—CH₂—, wherein R¹ and R² are each independently selected from —H, —COOH, —X, —NH₂, —CH₂NHC(NH)NH₂, —CX₃, —CH₃, —CH₂CH₃, and wherein X is selected from F, Cl, and Br;
      the method comprising contacting an amine compound according to structural Formula I

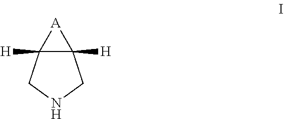

wherein A is as defined for structural Formula VII with oxygen in the presence of an engineered monoamine oxidase enzyme associated with a co-factor and with bisulfite under conditions which yield a mixture comprising a substantially enantiomerically pure aminosulfonate compound according to structural Formula III(a) and a substantially enantiomerically pure aminosulfonate compound according to structural Formula III(b)

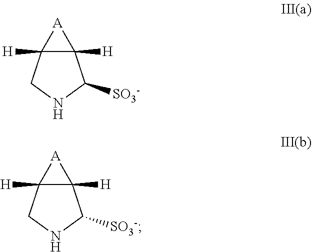

and
contacting a stereomerically pure bisulfite amine adduct compound according to structural Formula III(a), substantially stereomerically pure bisulfite amine adduct compound according to structural Formula III(b) or a mixture thereof with cyanide under conditions which yield the substantially stereomerically pure aminonitrile compound according to structural Formula IV(a)

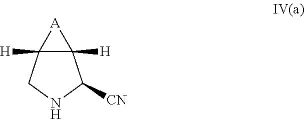

wherein A is as defined for the amino compound of structural Formula VII; and contacting the aminonitrile compound of structural Formula IV(a) with an acid under conditions in which the aminonitrile compound is converted to the substantially stereomerically pure amino amide compound according to structural Formula VII.

In another embodiment, any of the above processes involving the trans-aminonitrile compounds of Formula IV(a) can be carried out with the cis-aminonitrile compounds of Formula IV(b) to form the corresponding cis amino acid and amide products.

### Engineered Monoamine Oxidase Polypeptides

The present disclosure provides engineered monoamine polypeptides, polynucleotides encoding the polypeptides, methods of preparing the polypeptides, and methods for using the polypeptides. Where the description relates to polypeptides, it is to be understood that it can describe the polynucleotides encoding the polypeptides.

Suitable reaction conditions under which the above-described improved properties of the engineered polypeptides carry out the desired reaction can be determined with respect to concentrations or amounts of polypeptide, substrate, co-substrate, buffer, solvent, pH, conditions including temperature and reaction time, and/or conditions with the polypeptide immobilized on a solid support, as further described below and in the Examples.

In some embodiments, exemplary engineered monoamine oxidase polypeptides with improved properties, particularly in the conversion of bicyclic proline analogues of Formula I to the corresponding imines of Formula II, comprise an amino acid sequence that has one or more residue differences as compared to SEQ ID NOs: 6 and/or 414 at the residue positions indicated in Tables 4.2, 5.1, 6.1, 7.1, 8.1, and 9.1.

The structure and function information for exemplary non-naturally occurring (or engineered) polypeptides of the present invention are based on the conversion of bicyclic proline analogues of Formula I to the corresponding imines of Formula II, the results of which are shown below in Tables 4.2, 5.1, 6.1, 7.1, 8.1, and 9.1, as further described in the Examples. The odd numbered sequence identifiers (i.e., SEQ ID NOs) in these Tables refer to the nucleotide sequence encoding the amino acid sequence provided by the even numbered SEQ ID NOs in these Tables. Exemplary sequences are provided in the electronic sequence listing file accompanying this invention, which is hereby incorporated by reference herein. The amino acid residue differences are based on comparison to the reference sequence of SEQ ID NOs: 6 and/or 414, as indicated.

The previously engineered monoamine oxidase (SEQ ID NO: 6) derived from the monoamine oxidase of Aspergillus niger (SEQ ID NO: 2) and the monoamine oxidase of Aspergillus oryzae (SEQ ID NO: 4) was selected based on conversion of bicyclic proline analogues of Formula I to the corresponding imines of Formula II. SEQ ID NO: 5 is a codon-optimized polynucleotide for expression in Escherichia coli that was synthesized based on the polypeptide sequence of SEQ ID NO: 6.

The activity of each engineered monoamine oxidase polypeptide relative to the reference polypeptide of SEQ ID NOs: 6 and/or 414 was determined as conversion of the substrates described in the Examples herein. In some embodiments, a shake flask powder (SFP) is used as a secondary screen to assess the properties of the engineered monoamine oxidase polypeptides, the results of which are provided in the Examples. In some embodiments, the SFP forms provide a more purified powder preparation of the engineered polypeptides and can contain the engineered polypeptides that are up to about 30% of total protein.

In some embodiments, the specific enzyme properties are associated with the residues differences as compared to SEQ ID NOs: 6 and/or 414 at the residue positions indicated herein. In some embodiments, residue differences affecting polypeptide expression can be used to increase expression of the engineered monoamine oxidase polypeptides.

In light of the guidance provided herein, it is further contemplated that any of the exemplary engineered polypeptides comprising the even-numbered sequences of SEQ ID NOs: 6 and/or 414 find use as the starting amino acid sequence for synthesizing other engineered monoamine oxidase polypeptides, for example by subsequent rounds of evolution that incorporate new combinations of various amino acid differences from other polypeptides in Tables 4.2, 5.1, 6.1, 7.1, 8.1, and 9.1, and other residue positions described herein. Further improvements may be generated by including amino acid differences at residue positions that had been maintained as unchanged throughout earlier rounds of evolution.

In some embodiments, the engineered monoamine oxidase polypeptide comprises an amino acid sequence having at least 80%, 85%, 86%, 87%, 88%, 89%, 90%, 91%, 92%, 93%, 94%, 95%, 96%, 97%, 98%, 99% or more sequence identity to reference sequence SEQ ID NO: 6 and one or more residue differences as compared to SEQ ID NO: 6, selected from 13, 32/65/135/246/284, 32/135/284, 32/284, 47/197, 55/347/348, 61, 65/135, 65/190, 65/246, 65/284, 79, 80, 82, 85, 87, 87/185, 93, 100, 100/144/421/447, 100/144/447, 104, 107, 113, 114, 115, 117, 127, 135, 136, 144, 144/447, 145, 146, 147, 149, 149/157, 153, 155, 161, 162, 166, 167, 169, 170, 174, 177, 181, 184, 185, 187/242, 193, 196, 198, 199, 208, 211, 218, 219, 219/332, 221, 229, 232, 234, 238, 239, 240, 242, 246, 250, 252, 254, 260, 262/266/421, 262/421/447, 266/447, 277, 277/347, 284, 290, 292, 294, 298, 300, 306, 310, 323, 332, 338, 347, 349, 359, 361, 366, 381, 385, 386, 387, 392, 393, 400, 407, 411/445, 413, 418, 421, 425, 429, 433, 434, 441, 444, 445, 447, 449, 452, 475, and 483. In some embodiments, the engineered monoamine oxidase polypeptide comprises an amino acid sequence having at least 80%, 85%, 86%, 87%, 88%, 89%, 90%, 91%, 92%, 93%, 94%, 95%, 96%, 97%, 98%, 99% or more sequence identity to reference sequence SEQ ID NO: 6 and one or more residue differences as compared to SEQ ID NO: 6, selected from 13G, 32G/65V/135Q/246M/284D, 32G/135Q/284D, 32G/284D, 47A/197N, 55A/347D/348K, 61C, 65V/135Q, 65V/190D, 65V/246M, 65V/284D, 79A, 79D, 79Q, 79S, 79V, 79Y, 80H, 80I, 80N, 80T, 80V, 82F, 82L, 82R, 82W, 85D, 85S, 87G, 87H, 87L, 87Q/185F, 87S, 87W, 87Y, 93G, 100P, 100P/144E/421S/447A, 100P/144E/447A, 104K, 107W, 113G, 113W, 114G, 115M, 117A, 117C, 117I, 117K, 117L, 117R, 117S, 117T, 127D, 127G, 127R, 135G, 136R, 144E/447A, 144M, 145G, 145L, 145S, 146A, 146F, 147A, 149M, 149M/157A, 149V, 153T, 155C, 155L, 155Q, 155R, 161D, 161L, 161R, 161Y, 162Q, 166E, 166G, 166L, 167C, 169G, 170K, 174G, 174S, 174T, 174V, 177G, 177R, 177W, 181C, 181G, 181L, 181P, 184F, 184R, 185G, 185K, 185L, 185R, 187P/242I, 193A, 196G, 196M, 198D, 199I, 199T, 199V, 208S, 211G, 218G, 219P/332L, 219S, 221C, 221R, 221V, 229E, 232T, 234V, 238K, 238R, 239G, 240A, 242I, 246M, 250V, 252A, 252E, 252G, 252L, 254A, 260F, 260S, 260V, 262F/266L/421S, 262F/421S/447A, 266L/447A, 277T, 277T/347D, 284D, 290Q, 290Y, 292N, 294V, 298C, 300R, 306V, 310S, 323R, 332G, 332L, 332S, 338A, 338G, 338S, 347D, 347N, 349E, 359S, 361L, 366G, 381A, 385G, 386E, 386G, 387I, 387Q, 387W, 392G, 392S, 393G, 393K, 393M, 400D, 407G, 411V/445R, 413R, 418A, 418G, 421S, 425L, 429G, 433N, 433S, 434E, 441H, 444D, 444L, 444R, 444S, 445D, 445R, 447A, 449R, 449S, 452M, 475E, 475I, 475L, 475S, and 483R. In some embodiments, the engineered monoamine oxidase polypeptide comprises an amino acid sequence having at least 80%, 85%, 86%, 87%, 88%, 89%, 90%, 91%, 92%, 93%, 94%, 95%, 96%, 97%, 98%, 99% or more sequence identity to reference sequence SEQ ID NO: 6 and one or more residue differences as compared to SEQ ID NO: 6, selected from T13G, E32G/T65V/T135Q/I246M/N284D, E32G/T135Q/N284D, E32G/N284D, G47A/D197N, T55A/C347D/P348K, A61C, T65V/T135Q, T65V/E190D, T65V/I246M, T65V/N284D, W79A, W79D, W79Q, W79S, W79V, W79Y, S80H, S80I, S80N, S80T, S80V, N82F, N82L, N82R, N82W, G85D, G85S, P87G, P87H, P87L, P87Q/E185F, P87S, P87W, P87Y, T93G, S100P, S100P/A144E/C421S/C447A, S100P/A144E/C447A, R104K, T107W, N113G, N113W, A114G, L115M, P117A, P117C, P117I, P117K, P117L, P117R, P117S, P117T, H127D, H127G, H127R, T135G, T136R, A144E/C447A, A144M, E145G, E145L, E145S, D146A, D146F, E147A, L149M, L149M/T157A, L149V, L153T, K155C, K155L, K155Q, K155R, G161D, G161L, G161R, G161Y, T162Q, T166E, T166G, T166L, V167C, P169G, F170K, M174G, M174S, M174T, M174V, V177G, V177R, V177W, R181C, R181G, R181L, R181P, D184F, D184R, E185G, E185K, E185L, E185R, S187P/M242I, D193A, R196G, R196M, E198D, L199I, L199T, L199V, E208S, I211G, T218G, L219P/I332L, L219S, N221C, N221R, N221V, H229E, A232T, S234V, Y238K, Y238R, Q239G, G240A, M242I, 1246M, F250V, D252A, D252E, D252G, D252L, Q254A, R260F, R260S, R260V, W262F/A266L/C421S, W262F/C421S/C447A, A266L/C447A, C277T, C277T/C347D, N284D, R290Q, R290Y, T292N, R294V, E298C, A300R, C306V, L310S, S323R, I332G, I332L, I332S, C338A, C338G, C338S, C347D, C347N, D349E, P359S, N361L, A366G, C381A, A385G, H386E, H386G, N387I, N387Q, N387W, E392G, E392S, E393G, E393K, E393M, M400D, P407G, D411V/L445R, K413R, H418A, H418G, C421S, F425L, A429G, A433N, A433S, P434E, S441H, E444D, E444L, E444R, E444S, L445D, L445R, C447A, H449R, H449S, V452M, T475E, T475I, T475L, T475S, and E483R.

In some embodiments, the engineered monoamine oxidase polypeptide comprises an amino acid sequence having at least 80%, 85%, 86%, 87%, 88%, 89%, 90%, 91%, 92%, 93%, 94%, 95%, 96%, 97%, 98%, 99% or more sequence identity to reference sequence SEQ ID NO: 6 and one or more residue differences as compared to SEQ ID NO: 6, selected from 79, 80, 87, 146, 149, 219/332, 238, 260, 262/266/421, 338, and 429. In some embodiments, the engineered monoamine oxidase comprises an amino acid sequence having at least 80%, 85%, 86%, 87%, 88%, 89%, 90%, 91%, 92%, 93%, 94%, 95%, 96%, 97%, 98%, 99% or more sequence identity to reference sequence SEQ ID NO: 6 and one or more residue differences as compared to SEQ ID NO: 6, selected from 79D, 80V, 87G, 146A, 149M, 219P/332L, 238R, 260S, 262F/266L/421S, 338G, and 429G. In some embodiments, the engineered monoamine oxidase comprises an amino acid sequence having at least 80%, 85%, 86%, 87%, 88%, 89%, 90%, 91%, 92%, 93%, 94%, 95%, 96%, 97%, 98%, 99% or more sequence identity to reference sequence SEQ ID NO: 6 and one or more residue differences as compared to SEQ ID NO: 6, selected from W79D, S80V, P87G, D146A, L149M, L219P/I332L, Y238R, R260S, W262F/A266L/C421S, C338G, and A429G.

In some embodiments, the engineered monoamine oxidase comprises an amino acid sequence having at least 80%, 85%, 86%, 87%, 88%, 89%, 90%, 91%, 92%, 93%, 94%, 95%, 96%, 97%, 98%, 99% or more sequence identity to reference sequence SEQ ID NO: 6 and one or more residue differences as compared to SEQ ID NO: 6, selected from 79, 79/80, 79/80/332/429, 79/93, 79/93/238/386/429, 79/93/386, 79/183/284/332, 79/238, 79/238/332/429, 79/238/429, 80, 80/183/284/332/381/386/429, 80/188/190/332/386, 80/237/332, 80/238/332, 80/238/386/429, 80/284, 80/284/386, 80/332, 80/332/381/386, 80/332/386, 80/381, 80/386, 80/386/429, 80/429, 82, 82/87, 82/87/113, 82/87/113/117/218, 82/87/113/117/219, 82/87/113/117/219/418, 82/87/113/117/260/338, 82/87/113/117/338/418, 82/87/113/421, 82/87/117, 82/87/117/218/421, 82/87/117/260/338/421, 82/87/117/338/418, 82/87/218, 82/87/218/219/338, 82/87/218/219/421, 82/87/218/338, 82/87/219, 82/87/219/421, 82/87/338, 82/87/338/418, 82/87/338/421, 82/113/218/338, 82/113/218/338/418, 82/113/219/338, 82/113/338, 82/218/219/421, 82/260/338/421, 82/338/418/421, 82/418, 87, 87/113, 87/113/117/219/338/418/421, 87/113/117/260/338/421, 87/113/117/418/421, 87/113/218/219/260/338, 87/113/219/421, 87/113/260, 87/117, 87/117/218/219, 87/117/219/310/338, 87/218, 87/218/219, 87/218/219/338, 87/218/418, 87/219, 87/219/338, 87/219/418, 87/260, 87/310, 87/310/338, 87/338, 87/418, 87/421, 93/184/429, 93/238, 93/238/284, 93/238/386, 93/238/386/429, 100, 100/143/145/146/149/198/242/347/348/445/447, 100/143/145/149/347/447, 100/145/146/149, 100/145/146/149/154/348/445, 100/145/146/149/198, 100/145/146/149/198/242/250/252/347/348/447, 100/145/146/149/205/347/445/447, 100/145/146/149/242/445, 100/145/146/149/250/277/347/348/447, 100/145/149/154/246/277, 100/145/149/198/242/246/277/348/447, 100/145/149/198/242/246/445/447, 100/145/149/198/242/277/347/348/447, 100/145/149/198/252/348/445, 100/145/149/205, 100/145/149/242/246/277/347/445, 100/145/149/242/246/347/348/445/447, 100/145/149/242/445/447, 100/145/149/246/252/277/347/445/447, 100/145/149/246/347/445, 100/145/149/277/348/445, 100/145/149/347/445/447, 100/145/198/242/246/348, 100/145/198/277/348/445, 100/145/198/300, 100/145/205/246/277/348/445, 100/145/205/277/348, 100/145/242/246/277/445/447, 100/145/246/347/348/445, 100/145/277/347, 100/145/348, 100/146/149/198/242/246/277/347/348/445/447, 100/146/198/347/348/447, 100/146/198/445, 100/146/242/277/447, 100/146/250/447, 100/146/347/348/445/447, 100/149/198, 100/149/242/246/277/347/348/445, 100/149/242/246/277/347/348/447, 100/149/242/347/348/447, 100/149/246/277/348/445, 100/149/246/447, 100/149/250/347/447, 100/198/205/348, 100/198/277/347/348, 100/198/347/348, 100/198/347/348/445, 100/198/347/445, 100/198/447, 100/242/246/277, 100/242/246/347, 100/242/277/348, 100/242/347/348/445, 100/242/347/348/445/447, 100/242/348/445, 100/246/277/348/445, 100/277/347/445/447, 100/277/348, 100/277/348/447, 100/347/348/445, 100/347/447, 100/445/447, 110/338/418, 113/218/338/421, 113/338, 113/338/421, 117/219, 117/260, 121/198/347/445/447, 145/146/149/154/246/277/347/348, 145/146/149/198/242/252/277/347, 145/146/149/198/277/445/447, 145/146/149/205/348/447, 145/146/149/250/277/347/348/447, 145/146/149/277/347, 145/146/149/347/348, 145/146/198/205/242/277/348/447, 145/146/198/445/447, 145/146/246/277/447, 145/146/246/347/348/447, 145/146/277/347, 145/146/347/447, 145/149/198/205/348, 145/149/198/242/246/277/347/447, 145/149/198/242/246/348, 145/149/198/242/277/347, 145/149/198/246/447, 145/149/198/347/447, 145/149/205/242/347/348/445, 145/149/205/246/250/252, 145/149/205/277/445, 145/149/242, 145/149/242/348, 145/149/246, 145/149/246/347/445, 145/149/246/447, 145/149/347/348/445, 145/149/347/447, 145/149/447, 145/155/445/447, 145/198/205/348, 145/246, 145/246/252/347/447, 145/250/252/348, 145/252/348/447, 145/255, 145/277/347/445, 145/277/348/447, 145/347/348/445/447/491, 145/445/447, 146/149/151/246/348, 146/149/242/246/445, 146/149/246/252/447, 146/149/277/348/447, 146/198/246/277, 146/198/277/348/447, 146/242, 146/242/246/277/347/348/445/447, 146/246/277/348/445/447, 146/277, 146/277/347/348, 146/277/347/445/447, 146/277/445/447, 146/347/348/447, 146/348, 146/445/447, 149, 149/198/277/347/348, 149/198/348, 149/198/445, 149/242/246, 149/242/246/348/445, 149/347/348/445/447, 149/445, 184, 184/284, 184/284/429, 184/386, 198/242/348, 198/246, 198/250, 198/277/348/447, 198/445, 205, 205/246/347, 205/252/347/445/447, 205/347/348/445/447, 218, 218/260/338, 219, 219/338/418, 219/421, 238, 238/242/284, 238/284/386/429, 238/332/429, 238/381/429, 238/386, 238/429, 242, 242/277/347/348, 242/284/381/429, 242/348, 242/445/447, 246, 246/277/348/447, 246/445/447, 260, 260/338, 260/338/418/421, 277/347/348/445, 277/347/447, 284, 284/332, 284/386, 284/386/429, 310/338/418, 310/418/421, 332, 332/386, 332/429, 338, 338/418, 347/348/445, 347/348/447, 348, 348/445/447, 348/447, 386, 386/429, 418, 429, and 447. In some embodiments, the engineered monoamine oxidase comprises an amino acid sequence having at least 80%, 85%, 86%, 87%, 88%, 89%, 90%, 91%, 92%, 93%, 94%, 95%, 96%, 97%, 98%, 99% or more sequence identity to reference sequence SEQ ID NO: 6 and one or more residue differences as compared to SEQ ID NO: 6, selected from 79A/80H, 79A/93G/238R/386E/429G, 79A/93G/386E, 79A/238R, 79A/238R/332G/429G, 79A/238R/429G, 79D, 79D/80V, 79D/80V/332G/429G, 79D/93G, 79D/183F/284D/332G, 80H, 80H/183F/284D/332G/381A/386E/429G, 80H/188F/190D/332G/386E, 80H/238R/386E/429G, 80H/332G/381A/386E, 80H/381A, 80H/386E, 80H/429G, 80V, 80V/237S/332G, 80V/238R/332G, 80V/284D, 80V/284D/386E, 80V/332G, 80V/332G/386E, 80V/386E, 80V/386E/429G, 82F, 82F/87G/113G/117K/219S, 82F/87G/113G/117K/260S/338G, 82F/87G/117K, 82F/87G/218G, 82F/87G/218G/219S/421S, 82F/87G/218G/338S, 82F/87G/338S/418G, 82F/87L, 82F/87L/113G/117K/218G, 82F/87L/113G/117K/219S/418G, 82F/87L/113G/117K/338G/418G, 82F/87L/117K, 82F/87L/219S/421S, 82F/87L/338S, 82F/113G/218G/338S/418G, 82F/418G, 82R/87G, 82R/87G/113G/421S, 82R/87G/117K/218G/421S, 82R/87G/117K/260S/338S/421S, 82R/87G/117K/338A/418G, 82R/87L, 82R/87L/113G, 82R/87L/218G/219S/338S, 82R/87L/219S, 82R/87L/338G/421S, 82R/113G/218G/338A, 82R/113G/219S/338S, 82R/113G/338S, 82R/218G/219S/421S, 82R/260S/338A/421S, 82R/338A/418G/421S, 87G, 87G/113G, 87G/113G/117K/418G/421S, 87G/113G/260S, 87G/117K/219S/310S/338S, 87G/218G/219S/338S, 87G/219S, 87G/260S, 87G/310S, 87G/310S/338S, 87G/338A, 87G/418G, 87L, 87L/113G, 87L/113G/117K/219S/338G/418G/421S, 87L/113G/117K/260S/338S/421S, 87L/113G/218G/219S/260S/338A, 87L/113G/219S/421S, 87L/117K, 87L/117K/218G/219S, 87L/218G, 87L/218G/219S, 87L/218G/418G, 87L/219S, 87L/219S/338G, 87L/219S/418G, 87L/338A, 87L/421S, 93G/184R/429G, 93G/238R, 93G/238R/284D, 93G/238R/386E, 93G/238R/386E/429G, 100P, 100P/143D/145S/146A/149M/198D/242I/347N/348K/445R/447A, 100P/143V/145G/149M/347N/447A, 100P/145G/146A/149M/250V/277T/347N/348K/447A, 100P/145G/149M/154L/246M/277T, 100P/145G/149M/198D/242I/246M/445R/447A, 100P/145G/149M/198D/252G/348K/445R, 100P/145G/149M/205T, 100P/145G/149M/242I/246M/347D/348K/445R/447A, 100P/145G/149M/242I/445R/447A, 100P/145G/198D/242I/246M/348K, 100P/145G/198D/277T/348K/445R, 100P/145G/198D/300V, 100P/145G/205T/246M/277T/348K/445R, 100P/145G/242I/246M/277T/445R/447A, 100P/145G/348K, 100P/145S/146A/149M, 100P/145S/146A/149M/154L/348K/445R, 100P/145S/146A/149M/198D, 100P/145S/146A/149M/198D/242I/250V/252G/347N/348K/447A, 100P/145S/146A/149M/205T/347D/445R/447A, 100P/145S/146A/149M/242I/445R, 100P/145S/149M/198D/242I/246M/277T/348K/447A, 100P/145S/149M/198D/242I/277T/347N/348K/447A, 100P/145S/149M/242I/246M/277T/347N/445R, 100P/145S/149M/246M/252G/277T/347N/445R/447A, 100P/145S/149M/246M/347N/445R, 100P/145S/149M/277T/348K/445R, 100P/145S/149M/347N/445R/447A, 100P/145S/205T/277T/348K, 100P/145S/246M/347N/348K/445R, 100P/145S/277T/347N, 100P/146A/149M/198D/242I/246M/277T/347D/348K/445R/447A, 100P/146A/198D/347D/348K/447A, 100P/146A/198D/445R, 100P/146A/242I/277T/447A, 100P/146A/250V/447A, 100P/146A/347D/348K/445R/447A, 100P/149M/198D, 100P/149M/242I/246M/277T/347N/348K/445R, 100P/149M/242I/246M/277T/347N/348K/447A, 100P/149M/242I/347N/348K/447A, 100P/149M/246M/277T/348K/445R, 100P/149M/246M/447A, 100P/149M/250V/347N/447A, 100P/198D/205T/348K, 100P/198D/277T/347N/348K, 100P/198D/347D/348K, 100P/198D/347D/348K/445R, 100P/198D/347N/445R, 100P/198D/447A, 100P/242I/246M/277T, 100P/242I/246M/347N, 100P/242I/277T/348K, 100P/242I/347D/348K/445R, 100P/242I/347D/348K/445R/447A, 100P/242I/348K/445R, 100P/246M/277T/348K/445R, 100P/277T/347D/445R/447A, 100P/277T/348K, 100P/277T/348K/447A, 100P/347N/348K/445R, 100P/347N/447A, 100P/445R/447A, 110N/338G/418G, 113G/218G/338S/421S, 113G/338A/421S, 113G/338G, 117K/219S, 117K/260S, 121C/198D/347N/445R/447A, 145G/146A/149M/277T/347D, 145G/146A/149M/347D/348K, 145G/146A/277T/347N, 145G/146A/347D/447A, 145G/149M/198D/347N/447A, 145G/149M/205T/242I/347N/348K/445R, 145G/149M/205T/246M/250V/252G, 145G/149M/242I/348K, 145G/149M/246M, 145G/155I/445R/447A, 145G/198D/205T/348K, 145G/250V/252G/348K, 145G/255R, 145G/277T/347N/445R, 145S/146A/149M/154L/246M/277T/347N/348K, 145S/146A/149M/198D/242I/252G/277T/347N, 145S/146A/149M/198D/277T/445R/447A, 145S/146A/149M/205T/348K/447A, 145S/146A/149M/250V/277T/347D/348K/447A, 145S/146A/198D/205T/242I/277T/348K/447A, 145S/146A/198D/445R/447A, 145S/146A/246M/277T/447A, 145S/146A/246M/347N/348K/447A, 145S/149M/198D/205T/348K, 145S/149M/198D/242I/246M/277T/347N/447A, 145S/149M/198D/242I/246M/348K, 145S/149M/198D/242I/277T/347D, 145S/149M/198D/246M/447A, 145S/149M/205T/277T/445R, 145S/149M/242I, 145S/149M/246M/347D/445R, 145S/149M/246M/447A, 145S/149M/347N/348K/445R, 145S/149M/347N/447A, 145S/149M/447A, 145S/246M, 145S/246M/252G/347D/447A, 145S/252G/348K/447A, 145S/277T/348K/447A, 145S/347D/348K/445R/447A/491A, 145S/445R/447A, 146A/149M/151C/246M/348K, 146A/149M/242I/246M/445R, 146A/149M/246M/252G/447A, 146A/149M/277T/348K/447A, 146A/198D/246M/277T, 146A/198D/277T/348K/447A, 146A/242I, 146A/242I/246M/277T/347N/348K/445R/447A, 146A/246M/277T/348K/445R/447A, 146A/277T, 146A/277T/347N/348K, 146A/277T/347N/445R/447A, 146A/277T/445R/447A, 146A/347D/348K/447A, 146A/348K, 146A/445R/447A, 149M, 149M/198D/277T/347N/348K, 149M/198D/348K, 149M/198D/445R, 149M/242I/246M, 149M/242I/246M/348K/445R, 149M/347N/348K/445R/447A, 149M/445R, 184R, 184R/284D, 184R/284D/429G, 184R/386E, 198D/242I/348K, 198D/246M, 198D/250V, 198D/277T/348K/447A, 198D/445R, 205T, 205T/246M/347N, 205T/252G/347D/445R/447A, 205T/347D/348K/445R/447A, 218G, 218G/260S/338A, 2195, 2195/338A/418G, 219S/421S, 238R, 238R/242L/284D, 238R/284D/386E/429G, 238R/332G/429G, 238R/381A/429G, 238R/386E, 238R/429G, 242I, 242I/277T/347D/348K, 242I/348K, 242I/445R/447A, 242L/284D/381A/429G, 246M, 246M/277T/348K/447A, 246M/445R/447A, 260S, 260S/338G, 260S/338G/418G/421S, 277T/347N/348K/445R, 277T/347N/447A, 284D, 284D/332G, 284D/386E, 284D/386E/429G, 310S/338S/418G, 310S/418G/421S, 332G, 332G/386E, 332G/429G, 338G, 338G/418G, 347N/348K/445R, 347N/348K/447A, 348K, 348K/445R/447A, 348K/447A, 386E, 386E/429G, 418G, 429G, and 447A. In some embodiments, the engineered monoamine oxidase polypeptide comprises an amino acid sequence having at least 80%, 85%, 86%, 87%, 88%, 89%, 90%, 91%, 92%, 93%, 94%, 95%, 96%, 97%, 98%, 99% or more sequence identity to reference sequence SEQ ID NO: 6 and one or more residue differences as compared to SEQ ID NO: 6, selected from W79A/S80H, W79A/T93G/Y238R/H386E/A429G, W79A/T93G/H386E, W79A/Y238R, W79A/Y238R/I332G/A429G, W79A/Y238R/A429G, W79D, W79D/S80V, W79D/S80V/I332G/A429G, W79D/T93G, W79D/Y183F/N284D/I332G, S80H, S80H/Y183F/N284D/I332G/C381A/H386E/A429G, S80H/Y188F/E190D/I332G/H386E, S80H/Y238R/H386E/A429G, S80H/I332G/C381A/H386E, S80H/C381A, S80H/H386E, S80H/A429G, S80V, S80V/T237S/I332G, S80V/Y238R/I332G, S80V/N284D, S80V/N284D/H386E, S80V/I332G, S80V/I332G/H386E, S80V/H386E, S80V/H386E/A429G, N82F, N82F/P87G/N113G/P117K/L219S, N82F/P87G/N113G/P117K/R260S/C338G, N82F/P87G/P117K, N82F/P87G/T218G, N82F/P87G/T218G/L219S/C421S, N82F/P87G/T218G/C338S, N82F/P87G/C338S/H418G, N82F/P87L, N82F/P87L/N113G/P117K/T218G, N82F/P87L/N113G/P117K/L219S/H418G, N82F/P87L/N113G/P117K/C338G/H418G, N82F/P87L/P117K, N82F/P87L/L219S/C421S, N82F/P87L/C338S, N82F/N113G/T218G/C338S/H418G, N82F/H418G, N82R/P87G, N82R/P87G/N113G/C421S, N82R/P87G/P117K/T218G/C421S, N82R/P87G/P117K/R260S/C338S/C421S, N82R/P87G/P117K/C338A/H418G, N82R/P87L, N82R/P87L/N113G, N82R/P87L/T218G/L219S/C338S, N82R/P87L/L219S, N82R/P87L/C338G/C421S, N82R/N113G/T218G/C338A, N82R/N113G/L219S/C338S, N82R/N113G/C338S, N82R/T218G/L219S/C421S, N82R/R260S/C338A/C421S, N82R/C338A/H418G/C421S, P87G, P87G/N113G, P87G/N113G/P117K/H418G/C421S, P87G/N113G/R260S, P87G/P117K/L219S/L310S/C338S, P87G/T218G/L219S/C338S, P87G/L219S, P87G/R260S, P87G/L310S, P87G/L310S/C338S, P87G/C338A, P87G/H418G, P87L, P87L/N113G, P87L/N113G/P117K/L219S/C338G/H418G/C421S, P87L/N113G/P117K/R260S/C338S/C421S, P87L/N113G/T218G/L219S/R260S/C338A, P87L/N113G/L219S/C421S, P87L/P117K, P87L/P117K/T218G/L219S, P87L/T218G, P87L/T218G/L219S, P87L/T218G/H418G, P87L/L219S, P87L/L219S/C338G, P87L/L219S/H418G, P87L/C338A, P87L/C421S, T93G/D184R/A429G, T93G/Y238R, T93G/Y238R/N284D, T93G/Y238R/H386E, T93G/Y238R/H386E/A429G, S100P, S100P/E143D/E145S/D146A/L149M/E198D/M242I/C347N/P348K/L445R/C447A, S100P/E143V/E145G/L149M/C347N/C447A, S100P/E145G/D146A/L149M/F250V/C277T/C347N/P348K/C447A, S100P/E145G/L149M/H154L/I246M/C277T, S100P/E145G/L149M/E198D/M242I/I246M/L445R/C447A, S100P/E145G/L149M/E198D/D252G/P348K/L445R, S100P/E145G/L149M/S205T, S100P/E145G/L149M/M242U/246M/C347D/P348K/L445R/C447A, S100P/E145G/L149M/M242I/L445R/C447A, S100P/E145G/E198D/M242I/I246M/P348K, S100P/E145G/E198D/C277T/P348K/L445R, S100P/E145G/E198D/A300V, S100P/E145G/S205T/I246M/C277T/P348K/L445R, S100P/E145G/M242I/I246M/C277T/L445R/C447A, S100P/E145G/P348K, S100P/E145S/D146A/L149M, S100P/E145S/D146A/L149M/H154L/P348K/L445R, S100P/E145S/D146A/L149M/E198D, S100P/E145S/D146A/L149M/E198D/M242I/F250V/D252G/C347N/P348K/C447A, S100P/E145S/D146A/L149M/S205T/C347D/L445R/C447A, S100P/E145S/D146A/L149M/M242I/L445R, S100P/E145S/L149M/E198D/M242I/I246M/C277T/P348K/C447A, S100P/E145S/L149M/E198D/M242I/C277T/C347N/P348K/C447A, S100P/E145S/L149M/M242I/I246M/C277T/C347N/L445R, S100P/E145S/L149M/I246M/D252G/C277T/C347N/L445R/C447A, S100P/E145S/L149M/I246M/C347N/L445R, S100P/E145S/L149M/C277T/P348K/L445R, S100P/E145S/L149M/C347N/L445R/C447A, S100P/E145S/S205T/C277T/P348K, S100P/E145S/I246M/C347N/P348K/L445R, S100P/E145S/C277T/C347N, S100P/D146A/L149M/E198D/M242I/I246M/C277T/C347D/P348K/L445R/C447A, S100P/D146A/E198D/C347D/P348K/C447A, S100P/D146A/E198D/L445R, S100P/D146A/M242I/C277T/C447A, S100P/D146A/F250V/C447A, S100P/D146A/C347D/P348K/L445R/C447A, S100P/L149M/E198D, S100P/L149M/M242U/246M/C277T/C347N/P348K/L445R, S100P/L149M/M242U/246M/C277T/C347N/P348K/C447A, S100P/L149M/M242I/C347N/P348K/C447A, S100P/L149M/I246M/C277T/P348K/L445R, S100P/L149M/I246M/C447A, S100P/L149M/F250V/C347N/C447A, S100P/E198D/S205T/P348K, S100P/E198D/C277T/C347N/P348K, S100P/E198D/C347D/P348K, S100P/E198D/C347D/P348K/L445R, S100P/E198D/C347N/L445R, S100P/E198D/C447A, S100P/M242I/I246M/C277T, S100P/M242I/I246M/C347N, S100P/M242I/C277T/P348K, S100P/M242I/C347D/P348K/L445R, S100P/M242I/C347D/P348K/L445R/C447A, S100P/M242I/P348K/L445R, S100P/I246M/C277T/P348K/L445R, S100P/C277T/C347D/L445R/C447A, S100P/C277T/P348K, S100P/C277T/P348K/C447A, S100P/C347N/P348K/L445R, S100P/C347N/C447A, S100P/L445R/C447A, K110N/C338G/H418G, N113G/T218G/C338S/C421S, N113G/C338A/C421S, N113G/C338G, P117K/L219S, P117K/R260S, F121C/E198D/C347N/L445R/C447A, E145G/D146A/L149M/C277T/C347D, E145G/D146A/L149M/C347D/P348K, E145G/D146A/C277T/C347N, E145G/D146A/C347D/C447A, E145G/L149M/E198D/C347N/C447A, E145G/L149M/S205T/M242I/C347N/P348K/L445R, E145G/L149M/S205T/I246M/F250V/D252G, E145G/L149M/M242I/P348K, E145G/L149M/I246M, E145G/K155I/L445R/C447A, E145G/E198D/S205T/P348K, E145G/F250V/D252G/P348K, E145G/S255R, E145G/C277T/C347N/L445R, E145S/D146A/L149M/H154L/I246M/C277T/C347N/P348K, E145S/D146A/L149M/E198D/M242I/D252G/C277T/C347N, E145S/D146A/L149M/E198D/C277T/L445R/C447A, E145S/D146A/L149M/S205T/P348K/C447A, E145S/D146A/L149M/F250V/C277T/C347D/P348K/C447A, E145S/D146A/E198D/S205T/M242I/C277T/P348K/C447A, E145S/D146A/E198D/L445R/C447A, E145S/D146A/I246M/C277T/C447A, E145S/D146A/I246M/C347N/P348K/C447A, E145S/L149M/E198D/S205T/P348K, E145S/L149M/E198D/M242I/I246M/C277T/C347N/C447A, E145S/L149M/E198D/M242I/I246M/P348K, E145S/L149M/E198D/M242I/C277T/C347D, E145S/L149M/E198D/I246M/C447A, E145S/L149M/S205T/C277T/L445R, E145S/L149M/M242I, E145S/L149M/I246M/C347D/L445R, E145S/L149M/I246M/C447A, E145S/L149M/C347N/P348K/L445R, E145S/L149M/C347N/C447A, E145S/L149M/C447A, E145S/I246M, E145S/I246M/D252G/C347D/C447A, E145S/D252G/P348K/C447A, E145S/C277T/P348K/C447A, E145S/C347D/P348K/L445R/C447A/V491A, E145S/L445R/C447A, D146A/L149M/S151C/I246M/P348K, D146A/L149M/M242I/I246M/L445R, D146A/L149M/I246M/D252G/C447A, D146A/L149M/C277T/P348K/C447A, D146A/E198D/I246M/C277T, D146A/E198D/C277T/P348K/C447A, D146A/M242I, D146A/M242I/I246M/C277T/C347N/P348K/L445R/C447A, D146A/I246M/C277T/P348K/L445R/C447A, D146A/C277T, D146A/C277T/C347N/P348K, D146A/C277T/C347N/L445R/C447A, D146A/C277T/L445R/C447A, D146A/C347D/P348K/C447A, D146A/P348K, D146A/L445R/C447A, L149M, L149M/E198D/C277T/C347N/P348K, L149M/E198D/P348K, L149M/E198D/L445R, L149M/M242I/I246M, L149M/M242I/I246M/P348K/L445R, L149M/C347N/P348K/L445R/C447A, L149M/L445R, D184R, D184R/N284D, D184R/N284D/A429G, D184R/H386E, E198D/M242I/P348K, E198D/I246M, E198D/F250V, E198D/C277T/P348K/C447A, E198D/L445R, S205T, S205T/I246M/C347N, S205T/D252G/C347D/L445R/C447A, S205T/C347D/P348K/L445R/C447A, T218G, T218G/R260S/C338A, L219S, L219S/C338A/H418G, L219S/C421S, Y238R, Y238R/M242L/N284D, Y238R/N284D/H386E/A429G, Y238R/I332G/A429G, Y238R/C381A/A429G, Y238R/H386E, Y238R/A429G, M242I, M242I/C277T/C347D/P348K, M242I/P348K, M242I/L445R/C447A, M242L/N284D/C381A/A429G, I246M, I246M/C277T/P348K/C447A, I246M/L445R/C447A, R260S, R260S/C338G, R260S/C338G/H418G/C421S, C277T/C347N/P348K/L445R, C277T/C347N/C447A, N284D, N284D/I332G, N284D/H386E, N284D/H386E/A429G, L310S/C338S/H418G, L310S/H418G/C421S, I332G, I332G/H386E, I332G/A429G, C338G, C338G/H418G, C347N/P348K/L445R, C347N/P348K/C447A, P348K, P348K/L445R/C447A, P348K/C447A, H386E, H386E/A429G, H418G, A429G, and C447A.

In some embodiments, the engineered monoamine oxidase polypeptide comprises an amino acid sequence having at least 80%, 85%, 86%, 87%, 88%, 89%, 90%, 91%, 92%, 93%, 94%, 95%, 96%, 97%, 98%, 99% or more sequence identity to reference sequence SEQ ID NO: 6 and one or more residue differences as compared to SEQ ID NO: 6, selected from 80/429, 87/113/117/260/338/421, 87/421, 100/145/146/149/198/242/250/252/347/348/447, 100/145/149/198/242/277/347/348/447, 110/338/418, 145/149/205/246/250/252, 145/246/252/347/447, 145/252/348/447, 238/386/429, and 242/284/381/429. In some embodiments, the engineered monoamine oxidase comprises an amino acid sequence having at least 80%, 85%, 86%, 87%, 88%, 89%, 90%, 91%, 92%, 93%, 94%, 95%, 96%, 97%, 98%, 99% or more sequence identity to reference sequence SEQ ID NO: 6 and one or more residue differences as compared to SEQ ID NO: 6, selected from 80H/429G, 87L/113G/117K/260S/338S/421S, 87L/421S, 100P/145S/146A/149M/198D/242I/250V/252G/347N/348K/447A, 100P/145S/149M/198D/242I/277T/347N/348K/447A, 110N/338G/418G, 145G/149M/205T/246M/250V/252G, 145S/246M/252G/347D/447A, 145S/252G/348K/447A, 238R/386E/429G, and 242L/284D/381A/429G. In some embodiments, the engineered monoamine oxidase comprises an amino acid sequence having at least 80%, 85%, 86%, 87%, 88%, 89%, 90%, 91%, 92%, 93%, 94%, 95%, 96%, 97%, 98%, 99% or more sequence identity to reference sequence SEQ ID NO: 6 and one or more residue differences as compared to SEQ ID NO: 6, selected from S80H/A429G, P87L/N113G/P117K/R260S/C338S/C421S, P87L/C421S, S100P/E145S/D146A/L149M/E198D/M242I/F250V/D252G/C347N/P348K/C447A, S100P/E145S/L149M/E198D/M242I/C277T/C347N/P348K/C447A, K110N/C338G/H418G, E145G/L149M/S205T/I246M/F250V/D252G, E145S/I246M/D252G/C347D/C447A, E145S/D252G/P348K/C447A, Y238R/H386E/A429G, and M242L/N284D/C381A/A429G.

In some embodiments, the engineered monoamine oxidase polypeptide comprises an amino acid sequence having at least 80%, 85%, 86%, 87%, 88%, 89%, 90%, 91%, 92%, 93%, 94%, 95%, 96%, 97%, 98%, 99% or more sequence identity to reference sequence SEQ ID NO: 414 and one or more residue differences as compared to SEQ ID NO: 414, selected from 87/113/145/198/242/246/262/266, 87/117/149/242, 87/117/242/260/266, 87/145/149/198/447, 87/149/242/260/266, 87/149/260/262/266/381, 87/198, 87/242/246/260/262/266/338, 87/284/338/347, 113/117/146/149/260/262, 113/117/266/381, 113/117/447, 113/145/146/149/242/447, 113/242/347, 113/242/381, 117/145/146/262/284, 117/145/149/284/447, 117/149, 117/198, 117/198/262, 117/242/246/260/266/284/381, 117/242/260/262, 117/246/260/262/381, 117/246/284, 117/266, 145/146/149/260/262/338/347, 145/146/242/260/262/338/347/447, 145/149/198/260/266/338/347, 145/149/260/266/338/347/381/447, 146/149/260/262/347, 149/198/246/260/266/421, 198/447, 242, 242/246, 242/246/338/347, 242/260/262/338, 242/381, 246, 260/262/277/284/447, 260/266, 262, 347/381, and 447. In some embodiments, the engineered monoamine oxidase comprises an amino acid sequence having at least 80%, 85%, 86%, 87%, 88%, 89%, 90%, 91%, 92%, 93%, 94%, 95%, 96%, 97%, 98%, 99% or more sequence identity to reference sequence SEQ ID NO: 414 and one or more residue differences as compared to SEQ ID NO: 414, selected from 87G/113G/145L/198D/242I/246M/262F/266L, 87G/117K/149M/242I, 87G/145L/149M/198D/447A, 87G/149M/242I/260S/266L, 87L/117K/242I/260S/266L, 87L/149M/260S/262F/266L/381A, 87L/198D, 87L/242I/246M/260S/262F/266L/338G, 87L/284D/338G/347N, 113G/117K/146A/149M/260S/262F, 113G/117K/266L/381A, 113G/117K/447A, 113G/145L/146A/149M/242I/447A, 113G/242I/347N, 113G/242I/381A, 117K/145L/146A/262F/284D, 117K/145L/149M/284D/447A, 117K/149M, 117K/198D, 117K/198D/262F, 117K/242I/246M/260S/266L/284D/381A, 117K/242I/260S/262F, 117K/246M/260S/262F/381A, 117K/246M/284D, 117K/266L, 145L/146A/149M/260S/262F/338G/347N, 145L/146A/242I/260S/262F/338G/347N/447A, 145L/149M/198D/260S/266L/338G/347N, 145L/149M/260S/266L/338G/347N/381A/447A, 146A/149M/260S/262F/347N, 149M/198D/246M/260S/266L/421S, 198D/447A, 242I, 242I/246M, 242I/246M/338G/347N, 242I/260S/262F/338G, 242I/381A, 246M, 260S/262F/277T/284D/447A, 260S/266L, 262F, 347N/381A, and 447A. In some embodiments, the engineered monoamine oxidase comprises an amino acid sequence having at least 80%, 85%, 86%, 87%, 88%, 89%, 90%, 91%, 92%, 93%, 94%, 95%, 96%, 97%, 98%, 99% or more sequence identity to reference sequence SEQ ID NO: 414 and one or more residue differences as compared to SEQ ID NO: 414, selected from P87G/N113G/E145L/E198D/M242I/I246M/W262F/A266L, P87G/P117K/L149M/M242I, P87G/E145L/L149M/E198D/C447A, P87G/L149M/M242I/R260S/A266L, P87L/P117K/M242I/R260S/A266L, P87L/L149M/R260S/W262F/A266L/C381A, P87L/E198D, P87L/M242I/I246M/R260S/W262F/A266L/C338G, P87L/N284D/C338G/C347N, N113G/P117K/D146A/L149M/R260S/W262F, N113G/P117K/A266L/C381A, N113G/P117K/C447A, N113G/E145L/D146A/L149M/M242I/C447A, N113G/M242I/C347N, N113G/M242I/C381A, P117K/E145L/D146A/W262F/N284D, P117K/E145L/L149M/N284D/C447A, P117K/L149M, P117K/E198D, P117K/E198D/W262F, P117K/M242I/I246M/R260S/A266L/N284D/C381A, P117K/M242I/R260S/W262F, P117K/I246M/R260S/W262F/C381A, P117K/I246M/N284D, P117K/A266L, E145L/D146A/L149M/R260S/W262F/C338G/C347N, E145L/D146A/M242I/R260S/W262F/C338G/C347N/C447A, E145L/L149M/E198D/R260S/A266L/C338G/C347N, E145L/L149M/R260S/A266L/C338G/C347N/C381A/C447A, D146A/L149M/R260S/W262F/C347N, L149M/E198D/I246M/R260S/A266L/C421S, E198D/C447A, M242I, M242I/I246M, M242I/I246M/C338G/C347N, M242I/R260S/W262F/C338G, M242I/C381A, I246M, R260S/W262F/C277T/N284D/C447A, R260S/A266L, W262F, C347N/C381A, and C447A.

In some embodiments, the engineered monoamine oxidase polypeptide comprises an amino acid sequence having at least 80%, 85%, 86%, 87%, 88%, 89%, 90%, 91%, 92%, 93%, 94%, 95%, 96%, 97%, 98%, 99% or more sequence identity to reference sequence SEQ ID NO: 414 and one or more residue differences as compared to SEQ ID NO: 414, selected from 87/145/149/198/447, 87/149/260/262/266/381, 113/117/146/149/260/262, 117/145/149/284/447, 145/149/198/260/266/338/347, 146/149/260/262/347, and 149/198/246/260/266/421. In some embodiments, the engineered monoamine oxidase comprises an amino acid sequence having at least 80%, 85%, 86%, 87%, 88%, 89%, 90%, 91%, 92%, 93%, 94%, 95%, 96%, 97%, 98%, 99% or more sequence identity to reference sequence SEQ ID NO: 414 and one or more residue differences as compared to SEQ ID NO: 414, selected from 87G/145L/149M/198D/447A, 87L/149M/260S/262F/266L/381A, 113G/117K/146A/149M/260S/262F, 117K/145L/149M/284D/447A, 145L/149M/198D/260S/266L/338G/347N, 146A/149M/260S/262F/347N, and 149M/198D/246M/260S/266L/421S. In some embodiments, the engineered monoamine oxidase comprises an amino acid sequence having at least 80%, 85%, 86%, 87%, 88%, 89%, 90%, 91%, 92%, 93%, 94%, 95%, 96%, 97%, 98%, 99% or more sequence identity to reference sequence SEQ ID NO: 414 and one or more residue differences as compared to SEQ ID NO: 414, selected from P87G/E145L/L149M/E198D/C447A, P87L/L149M/R260S/W262F/A266L/C381A, N113G/P117K/D146A/L149M/R260S/W262F, P117K/E145L/L149M/N284D/C447A, E145L/L149M/E198D/R260S/A266L/C338G/C347N, D146A/L149M/R260S/W262F/C347N, and L149M/E198D/I246M/R260S/A266L/C421S.

As will be appreciated by the skilled artisan, in some embodiments, one or a combination of residue differences above that is selected can be kept constant (i.e., maintained) in the engineered monoamine oxidase as a core feature, and additional residue differences at other residue positions incorporated into the sequence to generate additional engineered monoamine oxidase polypeptides with improved properties. Accordingly, it is to be understood for any engineered monoamine oxidase containing one or a subset of the residue differences above, the present invention contemplates other engineered monoamine oxidases that comprise the one or subset of the residue differences, and additionally one or more residue differences at the other residue positions disclosed herein.

As noted above, the engineered monoamine oxidase polypeptides are also capable of converting substrates (e.g., Formula I or Compound 1) to products (e.g., Formula II or Compound 2). In some embodiments, the engineered monoamine oxidase polypeptide is capable of converting the substrate compounds to the product compound with at least 1.2 fold, 1.5 fold, 2 fold, 3 fold, 4 fold, 5 fold, 10 fold, 20 fold, 30 fold, 40 fold, 50 fold, 60 fold, 70 fold, 80 fold, 90 fold, 100 fold, or more activity relative to the activity of the reference polypeptide of SEQ ID NOs: 6 and/or 414.

In some embodiments, the engineered monoamine oxidase polypeptide capable of converting the substrate compounds to the product compounds with at least 2-fold the activity relative to SEQ ID NOs: 6 and/or 414, comprises an amino acid sequence selected from the even-numbered sequences in SEQ ID NOs: 6-1084.

In some embodiments, the engineered monoamine oxidase has an amino acid sequence comprising one or more residue differences as compared to SEQ ID NO: 6 or 414, increases expression of the engineered monoamine oxidase activity in a bacterial host cell, particularly in E. coli.

In some embodiments, the engineered monoamine oxidase has an amino acid sequence comprising one or more residue differences as compared to SEQ ID NO: 6 or 414, and decreases the enzyme loading necessary for conversion of a substrate to a product.

In some embodiments, the engineered monoamine oxidase has an amino acid sequence comprising one or more residue differences as compared to SEQ ID NO: 6 or 414, and decreases the enzyme loading necessary for conversion of a substrate to a product by about 10%, 25%, 33%, 50%, 75%, or more, as compared to a reference sequence of SEQ ID NO: 6 or 414.

In some embodiments, the engineered monoamine oxidase has an amino acid sequence comprising one or more residue differences as compared to SEQ ID NO: 6 or 414, wherein the enzyme loading of the engineered monoamine oxidase necessary for conversion of a substrate to a product is about 0.5% (w/w), 1% (w/w), 1.5% (w/w), 2% (w/w), 3% (w/w), 4% (w/w), 5% (w/w), 10% (w/w), or less of substrate compound loading.

In some embodiments, the engineered monoamine oxidase polypeptide with improved properties has an amino acid sequence comprising a sequence selected from the even-numbered sequences in the range of SEQ ID NOs: 6-1084.

In some embodiments, the engineered monoamine oxidase, comprises an amino acid sequence having at least 80%, 85%, 86%, 87%, 88%, 89%, 90%, 91%, 92%, 93%, 94%, 95%, 96%, 97%, 98%, or 99% identity to one of the even-numbered sequences in the range of SEQ ID NOs: 6-1084, and the amino acid residue differences as compared to SEQ ID NO: 6 or 414, present in any one of the even-numbered sequences in the range of SEQ ID NOs: 6-1084, as provided in the Examples.

In some embodiments, an engineered monoamine oxidase polypeptide of the disclosure comprises an amino acid sequence that is at least about 85%, 86%, 87%, 88%, 89%, 90%, 91%, 92%, 93%, 94%, 95%, 96%, 97%, 98%, or 99% identical to a reference sequence of SEQ ID NO: 6 or 414, wherein the polypeptide comprises an amino acid sequence where the amino acid residue corresponding to residue position 429 is glycine, wherein the residues are numbered with reference to the amino acid sequence of SEQ ID NO: 6 or 414. The modifications can include substitutions, deletions, and insertions. The substitutions can be non-conservative substitutions, conservative substitutions, or a combination of non-conservative and conservative substitutions.

In some embodiments, an engineered monoamine oxidase polypeptide of the disclosure comprises an amino acid sequence that is at least about 85%, 86%, 87%, 88%, 89%, 90%, 91%, 92%, 93%, 94%, 95%, 96%, 97%, 98%, or 99% identical to a reference sequence of SEQ ID NO: 6 or 414, wherein the polypeptide comprises an amino acid sequence where the amino acid residue corresponding to residue position 347 is cysteine or asparagine and the amino acid residue corresponding to residue position 429 is glycine, wherein the residues are numbered with reference to the amino acid sequence of SEQ ID NO: 6 or 414. The modifications can include substitutions, deletions, and insertions. The substitutions can be non-conservative substitutions, conservative substitutions, or a combination of non-conservative and conservative substitutions.

In some embodiments, an engineered monoamine oxidase polypeptide of the disclosure comprises an amino acid sequence that is at least about 85%, 86%, 87%, 88%, 89%, 90%, 91%, 92%, 93%, 94%, 95%, 96%, 97%, 98%, or 99% identical to a reference sequence of SEQ ID NO: 6 or 414, wherein the polypeptide comprises an amino acid sequence where the amino acid residue corresponding to residue position 421 is cysteine, the amino acid residue corresponding to residue position 347 is cysteine or asparagine, and the amino acid residue corresponding to residue position 429 is glycine, wherein the residues are numbered with reference to the amino acid sequence of SEQ ID NO: 6 or 414. The modifications can include substitutions, deletions, and insertions. The substitutions can be non-conservative substitutions, conservative substitutions, or a combination of non-conservative and conservative substitutions.

In some embodiments, an engineered monoamine oxidase polypeptide of the disclosure comprises an amino acid sequence that is at least about 85%, 86%, 87%, 88%, 89%, 90%, 91%, 92%, 93%, 94%, 95%, 96%, 97%, 98%, or 99% identical to a reference sequence of SEQ ID NO: 6 or 414, wherein the polypeptide comprises an amino acid sequence where the amino acid residue corresponding to residue position 145 is leucine, the amino acid residue corresponding to residue position 149 is methionine, the amino acid residue corresponding to residue position 198 is aspartic acid, the amino acid residue corresponding to residue position 260 is serine, the amino acid residue corresponding to residue position 266 is leucine, the amino acid residue corresponding to residue position 338 is glycine, the amino acid residue corresponding to residue position 347 is asparagine, and the amino acid residue corresponding to residue position 429 is glycine, wherein the residues are numbered with reference to the amino acid sequence of SEQ ID NO: 6 or 414. The modifications can include substitutions, deletions, and insertions.

The substitutions can be non-conservative substitutions, conservative substitutions, or a combination of non-conservative and conservative substitutions.

In some embodiments, an engineered monoamine oxidase polypeptide of the disclosure comprises an amino acid sequence that is at least about 85%, 86%, 87%, 88%, 89%, 90%, 91%, 92%, 93%, 94%, 95%, 96%, 97%, 98%, or 99 identical to a reference sequence of SEQ ID NO: 6 or 414, wherein the polypeptide comprises an amino acid sequence where the amino acid residue corresponding to residue position 87 is leucine, the amino acid residue corresponding to residue position 113 is glycine, the amino acid residue corresponding to residue position 117 is lysine, the amino acid residue corresponding to residue position 260 is serine, the amino acid residue corresponding to residue position 338 is serine, and the amino acid residue corresponding to residue position 421 is serine, wherein the residues are numbered with reference to the amino acid sequence of SEQ ID NO: 6 or 414. The modifications can include substitutions, deletions, and insertions. The substitutions can be non-conservative substitutions, conservative substitutions, or a combination of non-conservative and conservative substitutions.

In addition to the residue positions specified above, any of the engineered monoamine oxidase polypeptides disclosed herein can further comprise other residue differences relative to SEQ ID NOs: 6 and/or 414, at other residue positions (i.e., residue positions other than those included herein). Residue differences at these other residue positions can provide for additional variations in the amino acid sequence without adversely affecting the ability of the polypeptide to carry out the conversion of substrate to product. Accordingly, in some embodiments, in addition to the amino acid residue differences present in any one of the engineered monoamine oxidase polypeptides selected from the even-numbered sequences in the range of SEQ ID NOs: 6-1084, the sequence can further comprise 1-2, 1-3, 1-4, 1-5, 1-6, 1-7, 1-8, 1-9, 1-10, 1-11, 1-12, 1-14, 1-15, 1-16, 1-18, 1-20, 1-22, 1-24, 1-26, 1-30, 1-35, 1-40, 1-45, or 1-50 residue differences at other amino acid residue positions as compared to SEQ ID NOs: 6 and/or 414.

In some embodiments, the number of amino acid residue differences as compared to the reference sequence can be 1, 2, 3, 4, 5, 6, 7, 8, 9, 10, 11, 12, 13, 14, 15, 16, 17, 18, 19, 20, 21, 22, 23, 24, 25, 30, 30, 35, 40, 45 or 50 residue positions. In some embodiments, the number of amino acid residue differences as compared to the reference sequence can be 1, 2, 3, 4, 5, 6, 7, 8, 9, 10, 11, 12, 13, 14, 15, 16, 18, 20, 21, 22, 23, 24, or 25 residue positions. The residue differences at these other positions can be conservative changes or non-conservative changes. In some embodiments, the residue differences can comprise conservative substitutions and non-conservative substitutions as compared to the monoamine oxidase polypeptide of SEQ ID NO: 6 or 414.

In some embodiments, the present invention also provides engineered polypeptides that comprise a fragment of any of the engineered monoamine oxidase polypeptides described herein that retains the functional activity and/or improved property of that engineered monoamine oxidase. Accordingly, in some embodiments, the present invention provides a polypeptide fragment capable of converting substrate to product under suitable reaction conditions, wherein the fragment comprises at least about 90%, 95%, 96%, 97%, 98%, or 99% of a full-length amino acid sequence of an engineered monoamine oxidase of the present invention, such as an exemplary engineered monoamine oxidase polypeptide selected from the even-numbered sequences in the range of SEQ ID NOs: 6-1084. In some embodiments, the engineered monoamine oxidase can have an amino acid sequence comprising a deletion in any one of the monoamine oxidase polypeptide sequences described herein, such as the exemplary engineered polypeptides of the even-numbered sequences in the range of SEQ ID NOs: 6-1084.

Thus, for each and every embodiment of the engineered monoamine oxidase polypeptides of the invention, the amino acid sequence can comprise deletions of one or more amino acids, 2 or more amino acids, 3 or more amino acids, 4 or more amino acids, 5 or more amino acids, 6 or more amino acids, 8 or more amino acids, 10 or more amino acids, 15 or more amino acids, or 20 or more amino acids, up to 10% of the total number of amino acids, up to 20% of the total number of amino acids, or up to 30% of the total number of amino acids of the monoamine oxidase polypeptides, where the associated functional activity and/or improved properties of the engineered monoamine oxidase described herein are maintained. In some embodiments, the deletions can comprise 1-2, 1-3, 1-4, 1-5, 1-6, 1-7, 1-8, 1-9, 1-10, 1-15, 1-20, 1-21, 1-22, 1-23, 1-24, 1-25, 1-30, 1-35, 1-40, 1-45, or 1-50 amino acid residues. In some embodiments, the number of deletions can be 1, 2, 3, 4, 5, 6, 7, 8, 9, 10, 11, 12, 13, 14, 15, 16, 17, 18, 19, 20, 21, 22, 23, 24, 25, 30, 30, 35, 40, 45, or 50 amino acid residues. In some embodiments, the deletions can comprise deletions of 1, 2, 3, 4, 5, 6, 7, 8, 9, 10, 11, 12, 13, 14, 15, 16, 18, 20, 21, 22, 23, 24, or 25 amino acid residues.

In some embodiments, the engineered monoamine oxidase polypeptide described herein can have an amino acid sequence comprising an insertion as compared to any one of the engineered monoamine oxidase polypeptides described herein, such as the exemplary engineered polypeptides of the even-numbered sequences in the range of SEQ ID NOs: 6-1084. Thus, for each and every embodiment of the monoamine oxidase polypeptides of the disclosure, the insertions can comprise one or more amino acids, 2 or more amino acids, 3 or more amino acids, 4 or more amino acids, 5 or more amino acids, 6 or more amino acids, 8 or more amino acids, 10 or more amino acids, 15 or more amino acids, 20 or more amino acids, 30 or more amino acids, 40 or more amino acids, or 50 or more amino acids, where the associated functional activity and/or improved properties of the engineered monoamine oxidase described herein is maintained. The insertions can be to amino or carboxy terminus, or internal portions of the monoamine oxidase polypeptide.

In some embodiments, the engineered monoamine oxidase herein can have an amino acid sequence comprising a sequence selected from the even-numbered sequences in the range of SEQ ID NOs: 6-1084, and optionally one or several (e.g., up to 3, 4, 5, or up to 10) amino acid residue deletions, insertions and/or substitutions. In some embodiments, the amino acid sequence has optionally 1-2, 1-3, 1-4, 1-5, 1-6, 1-7, 1-8, 1-9, 1-10, 1-15, 1-20, 1-21, 1-22, 1-23, 1-24, 1-25, 1-30, 1-35, 1-40, 1-45, or 1-50 amino acid residue deletions, insertions and/or substitutions. In some embodiments, the number of amino acid sequence has optionally 1, 2, 3, 4, 5, 6, 7, 8, 9, 10, 11, 12, 13, 14, 15, 16, 17, 18, 19, 20, 21, 22, 23, 24, 25, 30, 30, 35, 40, 45, or 50 amino acid residue deletions, insertions and/or substitutions. In some embodiments, the amino acid sequence has optionally 1, 2, 3, 4, 5, 6, 7, 8, 9, 10, 11, 12, 13, 14, 15, 16, 18, 20, 21, 22, 23, 24, or 25 amino acid residue deletions, insertions and/or substitutions. In some embodiments, the substitutions can be conservative or non-conservative substitutions.

In the above embodiments, the suitable reaction conditions for the engineered polypeptides are provided in the Examples herein.

In some embodiments, the polypeptides of the present invention are fusion polypeptides in which the engineered polypeptides are fused to other polypeptides, such as, by way of example and not limitation, antibody tags (e.g., myc epitope), purification sequences (e.g., His tags for binding to metals), and cell localization signals (e.g., secretion signals). Thus, the engineered polypeptides described herein can be used with or without fusions to other polypeptides.

It is to be understood that the polypeptides described herein are not restricted to the genetically encoded amino acids. In addition to the genetically encoded amino acids, the polypeptides described herein may be comprised, either in whole or in part, of naturally occurring and/or synthetic non-encoded amino acids. Certain commonly encountered non-encoded amino acids of which the polypeptides described herein may be comprised include, but are not limited to: the D-stereomers of the genetically-encoded amino acids; 2,3-diaminopropionic acid (Dpr); α-aminoisobutyric acid (Aib); ε-aminohexanoic acid (Aha); 8-aminovaleric acid (Ava); N-methylglycine or sarcosine (MeGly or Sar); ornithine (Orn); citrulline (Cit); t-butylalanine (Bua); t-butylglycine (Bug); N-methylisoleucine (MeIle); phenylglycine (Phg); cyclohexylalanine (Cha); norleucine (Nle); naphthylalanine (Nal); 2-chlorophenylalanine (Ocf); 3-chlorophenylalanine (Mcf); 4-chlorophenylalanine (Pcf); 2-fluorophenylalanine (Off); 3-fluorophenylalanine (Mff); 4-fluorophenylalanine (Pff); 2-bromophenylalanine (Obf); 3-bromophenylalanine (Mbf); 4-bromophenylalanine (Pbf); 2-methylphenylalanine (Omf); 3-methylphenylalanine (Mmf); 4-methylphenylalanine (Pmf); 2-nitrophenylalanine (Onf); 3-nitrophenylalanine (Mnf); 4-nitrophenylalanine (Pnf); 2-cyanophenylalanine (Ocf); 3-cyanophenylalanine (Mcf); 4-cyanophenylalanine (Pcf); 2-trifluoromethylphenylalanine (Otf); 3-trifluoromethylphenylalanine (Mtf); 4-trifluoromethylphenylalanine (Ptf); 4-aminophenylalanine (Paf); 4-iodophenylalanine (Pif); 4-aminomethylphenylalanine (Pamf); 2,4-dichlorophenylalanine (Opef); 3,4-dichlorophenylalanine (Mpcf); 2,4-difluorophenylalanine (Opff); 3,4-difluorophenylalanine (Mpff); pyrid-2-ylalanine (2pAla); pyrid-3-ylalanine (3pAla); pyrid-4-ylalanine (4pAla); naphth-1-ylalanine (1nAla); naphth-2-ylalanine (2nAla); thiazolylalanine (taAla); benzothienylalanine (bAla); thienylalanine (tAla); furylalanine (fAla); homophenylalanine (hPhe); homotyrosine (hTyr); homotryptophan (hTrp); pentafluorophenylalanine (5ff); styrylkalanine (sAla); authrylalanine (aAla); 3,3-diphenylalanine (Dfa); 3-amino-5-phenypentanoic acid (Afp); penicillamine (Pen); 1,2,3,4-tetrahydroisoquinoline-3-carboxylic acid (Tic); β-2-thienylalanine (Thi); methionine sulfoxide (Mso); N(w)-nitroarginine (nArg); homolysine (hLys); phosphonomethylphenylalanine (pmPhe); phosphoserine (pSer); phosphothreonine (pThr); homoaspartic acid (hAsp); homoglutanic acid (hGlu); 1-aminocyclopent-(2 or 3)-ene-4 carboxylic acid; pipecolic acid (PA), azetidine-3-carboxylic acid (ACA); 1-aminocyclopentane-3-carboxylic acid; allylglycine (aGly); propargylglycine (pgGly); homoalanine (hAla); norvaline (nVal); homoleucine (hLeu), homovaline (hVal); homoisoleucine (hIle); homoarginine (hArg); N-acetyl lysine (AcLys); 2,4-diaminobutyric acid (Dbu); 2,3-diaminobutyric acid (Dab); N-methylvaline (MeVal); homocysteine (hCys); homoserine (hSer); hydroxyproline (Hyp) and homoproline (hPro). Additional non-encoded amino acids of which the polypeptides described herein may be comprised will be apparent to those of skill in the art (See e.g., the various amino acids provided in Fasman, CRC Practical Handbook of Biochemistry and Molecular Biology, CRC Press, Boca Raton, FL, pp. 3-70 [1989], and the references cited therein, all of which are incorporated by reference). These amino acids may be in either the L- or D-configuration.

Those of skill in the art will recognize that amino acids or residues bearing side chain protecting groups may also comprise the polypeptides described herein. Non-limiting examples of such protected amino acids, which in this case belong to the aromatic category, include (protecting groups listed in parentheses), but are not limited to: Arg(tos), Cys(methylbenzyl), Cys (nitropyridinesulfenyl), Glu(8-benzylester), Gln(xanthyl), Asn(N-8-xanthyl), His(bom), His(benzyl), His(tos), Lys(fmoc), Lys(tos), Ser(O-benzyl), Thr (O-benzyl) and Tyr(O-benzyl).

Non-encoding amino acids that are conformationally constrained of which the polypeptides described herein may be composed include, but are not limited to, N-methyl amino acids (L-configuration); 1-aminocyclopent-(2 or 3)-ene-4-carboxylic acid; pipecolic acid; azetidine-3-carboxylic acid; homoproline (hPro); and 1-aminocyclopentane-3-carboxylic acid.

In some embodiments, the engineered polypeptides can be in various forms, for example, such as an isolated preparation, as a substantially purified enzyme, whole cells transformed with gene(s) encoding the enzyme, and/or as cell extracts and/or lysates of such cells. The enzymes can be lyophilized, spray-dried, precipitated or be in the form of a crude paste, as further discussed below.

In some embodiments, the engineered polypeptides can be in the form of a biocatalytic composition. In some embodiments, the biocatalytic composition comprises (a) a means for conversion of a fused bicyclic proline analogue compound of Formula I to a corresponding imine of Formula II by contact with a monoamine oxidase polypeptide and (b) a suitable cofactor in the presence of oxygen. In some embodiments, the biocatalytic composition comprises a FAD cofactor. In some embodiments, the biocatalytic composition comprises catalase. In some embodiments, the composition further comprises NaHSO₃. In some embodiments, the composition further comprises HCN.

In some embodiments, the engineered polypeptides can be provided on a solid support, such as a membrane, resin, solid carrier, or other solid phase material. A solid support can be composed of organic polymers such as polystyrene, polyethylene, polypropylene, polyfluoroethylene, polyethyleneoxy, and polyacrylamide, as well as co-polymers and grafts thereof. A solid support can also be inorganic, such as glass, silica, controlled pore glass (CPG), reverse phase silica or metal, such as gold or platinum. The configuration of a solid support can be in the form of beads, spheres, particles, granules, a gel, a membrane or a surface. Surfaces can be planar, substantially planar, or non-planar. Solid supports can be porous or non-porous, and can have swelling or non-swelling characteristics. A solid support can be configured in the form of a well, depression, or other container, vessel, feature, or location.

In some embodiments, the engineered monoamine oxidase polypeptides of the present invention can be immobilized on a solid support such that they retain their improved activity, and/or other improved properties relative to the reference polypeptide of SEQ ID NO: 6 or 414. In such embodiments, the immobilized polypeptides can facilitate the biocatalytic conversion of the substrate compounds or other suitable substrates to the product and after the reaction is complete are easily retained (e.g., by retaining beads on which polypeptide is immobilized) and then reused or recycled in subsequent reactions. Such immobilized enzyme processes allow for further efficiency and cost reduction. Accordingly, it is further contemplated that any of the methods of using the monoamine oxidase polypeptides of the present invention can be carried out using the monoamine oxidase polypeptides bound or immobilized on a solid support.

Methods of enzyme immobilization are well-known in the art. The engineered polypeptides can be bound non-covalently or covalently. Various methods for conjugation and immobilization of enzymes to solid supports (e.g., resins, membranes, beads, glass, etc.) are well known in the art (See e.g., Yi et al., Proc. Biochem., 42(5): 895-898 [2007]; Martin et al., Appl. Microbiol. Biotechnol., 76(4): 843-851 [2007]; Koszelewski et al., J. Mol. Cat. B: Enzymatic, 63: 39-44 [2010]; Truppo et al., Org. Proc. Res. Dev., published online: dx.doi.org/10.1021/op200157c; Hermanson, Bioconjugate Techniques, 2^{nd} ed., Academic Press, Cambridge, MA [2008]; Mateo et al., Biotechnol. Prog., 18(3):629-34 [2002]; and “Bioconjugation Protocols: Strategies and Methods,” In Methods in Molecular Biology, Niemeyer (ed.), Humana Press, New York, NY [2004]; the disclosures of each which are incorporated by reference herein). Solid supports useful for immobilizing the engineered monoamine oxidase of the present invention include but are not limited to beads or resins comprising polymethacrylate with epoxide functional groups, polymethacrylate with amino epoxide functional groups, styrene/DVB copolymer or polymethacrylate with octadecyl functional groups. Exemplary solid supports useful for immobilizing the engineered monoamine oxidase polypeptides of the present invention include, but are not limited to, chitosan beads, Eupergit C, and SEPABEADs (Mitsubishi), including the following different types of SEPABEAD: EC-EP, EC-HFA/S, EXA252, EXE119 and EXE120.

In some embodiments, the polypeptides described herein are provided in the form of kits. The enzymes in the kits may be present individually or as a plurality of enzymes. The kits can further include reagents for carrying out the enzymatic reactions, substrates for assessing the activity of enzymes, as well as reagents for detecting the products. The kits can also include reagent dispensers and instructions for use of the kits.

In some embodiments, the kits of the present invention include arrays comprising a plurality of different monoamine oxidase polypeptides at different addressable position, wherein the different polypeptides are different variants of a reference sequence each having at least one different improved enzyme property. In some embodiments, a plurality of polypeptides immobilized on solid supports are configured on an array at various locations, addressable for robotic delivery of reagents, or by detection methods and/or instruments. The array can be used to test a variety of substrate compounds for conversion by the polypeptides. Such arrays comprising a plurality of engineered polypeptides and methods of their use are known in the art (See e.g., WO2009/008908A2).

### Polynucleotides Encoding Engineered Monoamine Oxidases, Expression Vectors and Host Cells

In another aspect, the present invention provides polynucleotides encoding the engineered monoamine oxidase polypeptides described herein. The polynucleotides may be operatively linked to one or more heterologous regulatory sequences that control gene expression to create a recombinant polynucleotide capable of expressing the polypeptide. Expression constructs containing a heterologous polynucleotide encoding the engineered monoamine oxidase are introduced into appropriate host cells to express the corresponding monoamine oxidase polypeptide.

As will be apparent to the skilled artisan, availability of a protein sequence and the knowledge of the codons corresponding to the various amino acids provide a description of all the polynucleotides capable of encoding the subject polypeptides. The degeneracy of the genetic code, where the same amino acids are encoded by alternative or synonymous codons, allows an extremely large number of nucleic acids to be made, all of which encode the improved monoamine oxidase enzymes. Thus, having knowledge of a particular amino acid sequence, those skilled in the art could make any number of different nucleic acids by simply modifying the sequence of one or more codons in a way which does not change the amino acid sequence of the protein. In this regard, the present invention specifically contemplates each and every possible variation of polynucleotides that could be made encoding the polypeptides described herein by selecting combinations based on the possible codon choices, and all such variations are to be considered specifically disclosed for any polypeptide described herein, including the amino acid sequences presented in Tables 4.2, 5.1, 6.1, 7.1, 8.1, and 9.1, and disclosed in the sequence listing incorporated by reference herein as the even-numbered sequences in the range of SEQ ID NOs: 6-1084.

In various embodiments, the codons are preferably selected to fit the host cell in which the protein is being produced. For example, preferred codons used in bacteria are used to express the gene in bacteria; preferred codons used in yeast are used for expression in yeast; and preferred codons used in mammals are used for expression in mammalian cells. In some embodiments, all codons need not be replaced to optimize the codon usage of the monoamine oxidase since the natural sequence will comprise preferred codons and because use of preferred codons may not be required for all amino acid residues.

Consequently, codon optimized polynucleotides encoding the monoamine oxidase enzymes may contain preferred codons at about 40%, 50%, 60%, 70%, 80%, or greater than 90% of codon positions of the full length coding region.

In some embodiments, the polynucleotide comprises a codon optimized nucleotide sequence encoding the monoamine oxidase polypeptide amino acid sequence, as represented by SEQ ID NOs: 5 and/or 413. In some embodiments, the polynucleotide has a nucleic acid sequence comprising at least 80%, 85%, 90%, 91%, 92%, 93%, 94%, 95%, 96%, 97%, 98%, 99% or more identity to the codon optimized nucleic acid sequences encoding the even-numbered sequences in the range of SEQ ID NOs: 6-1084. In some embodiments, the polynucleotide has a nucleic acid sequence comprising at least 80%, 85%, 90%, 91%, 92%, 93%, 94%, 95%, 96%, 97%, 98%, 99% or more identity to the codon optimized nucleic acid sequences in the odd-numbered sequences in the range of SEQ ID NOs: 5-1083. In some embodiments, the codon optimized sequences of the odd-numbered sequences in the range of SEQ ID NOs: 5-1083, enhance expression of the encoded monoamine oxidase, providing preparations of enzyme capable of converting substrate to product.

In some embodiments, the polynucleotides are capable of hybridizing under highly stringent conditions to a reference sequence selected from the odd-numbered sequences in SEQ ID NOs: 5-1083, or a complement thereof, and encode a monoamine oxidase polypeptide.

In some embodiments, as described above, the polynucleotide encodes an engineered monoamine oxidase polypeptide with improved properties as compared to SEQ ID NO: 6 or 414, wherein the polypeptide comprises an amino acid sequence having at least 80%, 85%, 86%, 87%, 88%, 89%, 90%, 91%, 92%, 93%, 94%, 95%, 96%, 97%, 98%, 99% or more identity to a reference sequence selected from SEQ ID NO: 6 or 414, and one or more residue differences as compared to SEQ ID NO: 6 or 414, wherein the sequence is selected from the even-numbered sequences in the range of SEQ ID NOs: 6-1084. In some embodiments, the reference amino acid sequence is selected from the even-numbered sequences in the range of SEQ ID NOs: 6-1084. In some embodiments, the reference amino acid sequence is SEQ ID NO: 6, while in some other embodiments, the reference sequence is SEQ ID NO: 414.

In some embodiments, the polynucleotide encodes a monoamine oxidase polypeptide capable of converting one or more substrates to product with improved properties as compared to SEQ ID NO: 6 or 414, wherein the polypeptide comprises an amino acid sequence having at least 80%, 85%, 86%, 87%, 88%, 89%, 90%, 91%, 92%, 93%, 94%, 95%, 96%, 97%, 98%, 99% or more sequence identity to reference sequence SEQ ID NO: 6 or 414.

In some embodiments, the polynucleotide encoding the engineered monoamine oxidase comprises a polynucleotide sequence selected from the odd-numbered sequences in the range of SEQ ID NOs: 5-1083.

In some embodiments, the polynucleotides are capable of hybridizing under highly stringent conditions to a reference polynucleotide sequence selected from the odd-numbered sequences in the range of SEQ ID NOs: 5-1083, or a complement thereof, and encode a monoamine oxidase polypeptide with one or more of the improved properties described herein. In some embodiments, the polynucleotide capable of hybridizing under highly stringent conditions encodes a monoamine oxidase comprising an amino acid sequence having at least 80%, 85%, 86%, 87%, 88%, 89%, 90%, 91%, 92%, 93%, 94%, 95%, 96%, 97%, 98%, 99% or more identity to any of the even-numbered sequences of SEQ ID NOs: 6-1084, that has an amino acid sequence comprising one or more residue differences as compared to SEQ ID NOs: 6 and/or 414.

In some embodiments, the polynucleotide capable of hybridizing under highly stringent conditions encodes an engineered monoamine oxidase with improved properties comprising an amino acid sequence having at least 80%, 85%, 86%, 87%, 88%, 89%, 90%, 91%, 92%, 93%, 94%, 95%, 96%, 97%, 98%, 99% or more identity to SEQ ID NO: 6 or 414. In some embodiments, the polynucleotides encode the polypeptides described herein but have at least about 80%, 85%, 86%, 87%, 88%, 89%, 90%, 91%, 92%, 93%, 94%, 95%, 96%, 97%, 98%, or 99% or more sequence identity at the nucleotide level to a reference polynucleotide encoding the engineered monoamine oxidase. In some embodiments, the reference polynucleotide sequence is selected from SEQ ID NOs: 5-1083.

In some embodiments, the polynucleotide capable of hybridizing under highly stringent conditions encodes an engineered monoamine oxidase polypeptide with improved properties comprising an amino acid sequence having at least 80%, 85%, 86%, 87%, 88%, 89%, 90%, 91%, 92%, 93%, 94%, 95%, 96%, 97%, 98%, 99% or more identity to SEQ ID NO: 6. In some embodiments, the polynucleotides encode the polypeptides described herein but have at least about 80%, 85%, 86%, 87%, 88%, 89%, 90%, 91%, 92%, 93%, 94%, 95%, 96%, 97%, 98%, or 99% or more sequence identity at the nucleotide level to a reference polynucleotide encoding the engineered monoamine oxidase. In some embodiments, the reference polynucleotide sequence is selected from SEQ ID NOs: 5-993.

In some embodiments, the polynucleotide capable of hybridizing under highly stringent conditions encodes an engineered monoamine oxidase polypeptide with improved properties comprising an amino acid sequence having at least 80%, 85%, 86%, 87%, 88%, 89%, 90%, 91%, 92%, 93%, 94%, 95%, 96%, 97%, 98%, 99% or more identity to SEQ ID NO: 414. In some embodiments, the polynucleotides encode the polypeptides described herein but have at least about 80%, 85%, 86%, 87%, 88%, 89%, 90%, 91%, 92%, 93%, 94%, 95%, 96%, 97%, 98%, or 99% or more sequence identity at the nucleotide level to a reference polynucleotide encoding the engineered monoamine oxidase. In some embodiments, the reference polynucleotide sequence is selected from SEQ ID NOs: 995-1083.

In some embodiments, an isolated polynucleotide encoding any of the engineered monoamine oxidase polypeptides provided herein is manipulated in a variety of ways to provide for expression of the polypeptide. In some embodiments, the polynucleotides encoding the polypeptides are provided as expression vectors where one or more control sequences is present to regulate the expression of the polynucleotides and/or polypeptides. Manipulation of the isolated polynucleotide prior to its insertion into a vector may be desirable or necessary depending on the expression vector. The techniques for modifying polynucleotides and nucleic acid sequences utilizing recombinant DNA methods are well known in the art.

In some embodiments, the control sequences include among other sequences, promoters, leader sequences, polyadenylation sequences, propeptide sequences, signal peptide sequences, and transcription terminators. As known in the art, suitable promoters can be selected based on the host cells used. For bacterial host cells, suitable promoters for directing transcription of the nucleic acid constructs of the present application, include, but are not limited to the promoters obtained from the E. coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xylA and xylB genes, and prokaryotic beta-lactamase gene (See e.g., Villa-Kamaroff et al., Proc. Natl Acad. Sci. USA 75: 3727-3731 [1978]), as well as the tac promoter (See e.g., DeBoer et al., Proc. Natl Acad. Sci. USA 80: 21-25 [1983]). Exemplary promoters for filamentous fungal host cells, include promoters obtained from the genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, and Fusarium oxysporum trypsin-like protease (See e.g., WO 96/00787), as well as the NA2-tpi promoter (a hybrid of the promoters from the genes for Aspergillus niger neutral alpha-amylase and Aspergillus oryzae triose phosphate isomerase), and mutant, truncated, and hybrid promoters thereof.

Exemplary yeast cell promoters can be from the genes can be from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH2/GAP), and Saccharomyces cerevisiae 3-phosphoglycerate kinase. Other useful promoters for yeast host cells are known in the art (See e.g., Romanos et al., Yeast 8:423-488 [1992]).

In some embodiments, the control sequence is a suitable transcription terminator sequence, a sequence recognized by a host cell to terminate transcription. The terminator sequence is operably linked to the 3′ terminus of the nucleic acid sequence encoding the polypeptide. Any terminator which is functional in the host cell of choice finds use in the present invention. For example, exemplary transcription terminators for filamentous fungal host cells can be obtained from the genes for Aspergillus oryzae TAKA amylase, Aspergillus niger glucoamylase, Aspergillus nidulans anthranilate synthase, Aspergillus niger alpha-glucosidase, and Fusarium oxysporum trypsin-like protease. Exemplary terminators for yeast host cells can be obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are known in the art (See e.g., Romanos et al., supra).

In some embodiments, the control sequence is a suitable leader sequence, a non-translated region of an mRNA that is important for translation by the host cell. The leader sequence is operably linked to the 5′ terminus of the nucleic acid sequence encoding the polypeptide. Any leader sequence that is functional in the host cell of choice may be used. Exemplary leaders for filamentous fungal host cells are obtained from the genes for Aspergillus oryzae TAKA amylase and Aspergillus nidulans triose phosphate isomerase. Suitable leaders for yeast host cells include, but are not limited to those obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP). The control sequence may also be a polyadenylation sequence, a sequence operably linked to the 3′ terminus of the nucleic acid sequence and which, when transcribed, is recognized by the host cell as a signal to add polyadenosine residues to transcribed mRNA. Any polyadenylation sequence which is functional in the host cell of choice may be used in the present invention. Exemplary polyadenylation sequences for filamentous fungal host cells include, but are not limited to those from the genes for Aspergillus oryzae TAKA amylase, Aspergillus niger glucoamylase, Aspergillus nidulans anthranilate synthase, Fusarium oxysporum trypsin-like protease, and Aspergillus niger alpha-glucosidase. Useful polyadenylation sequences for yeast host cells are also known in the art (See e.g., Guo and Sherman, Mol. Cell. Bio., 15:5983-5990 [1995]).

In some embodiments, the control sequence is a signal peptide coding region that codes for an amino acid sequence linked to the amino terminus of a polypeptide and directs the encoded polypeptide into the cell's secretory pathway. The 5′ end of the coding sequence of the nucleic acid sequence may inherently contain a signal peptide coding region naturally linked in translation reading frame with the segment of the coding region that encodes the secreted polypeptide. Alternatively, the 5′ end of the coding sequence may contain a signal peptide coding region that is foreign to the coding sequence. Any signal peptide coding region that directs the expressed polypeptide into the secretory pathway of a host cell of choice finds use for expression of the engineered monoamine oxidase polypeptides provided herein. Effective signal peptide coding regions for bacterial host cells include, but are not limited to the signal peptide coding regions obtained from the genes for Bacillus NClB 11837 maltogenic amylase, Bacillus stearothermophilus alpha-amylase, Bacillus licheniformis subtilisin, Bacillus licheniformis beta-lactamase, Bacillus stearothermophilus neutral proteases (nprT, nprS, nprM), and Bacillus subtilis prsA.

Further signal peptides are known in the art (See e.g., Simonen and Palva, Microbiol. Rev., 57:109-137 [1993]). Effective signal peptide coding regions for filamentous fungal host cells include, but are not limited to the signal peptide coding regions obtained from the genes for Aspergillus oryzae TAKA amylase, Aspergillus niger neutral amylase, Aspergillus niger glucoamylase, Rhizomucor miehei aspartic proteinase, Humicola insolens cellulase, and Humicola lanuginosa lipase. Useful signal peptides for yeast host cells include, but are not limited to those from the genes for Saccharomyces cerevisiae alpha-factor and Saccharomyces cerevisiae invertase.

In some embodiments, the control sequence is a propeptide coding region that codes for an amino acid sequence positioned at the amino terminus of a polypeptide. The resultant polypeptide is referred to as a “proenzyme,” “propolypeptide,” or “zymogen,” in some cases). A propolypeptide can be converted to a mature active polypeptide by catalytic or autocatalytic cleavage of the propeptide from the propolypeptide. The propeptide coding region includes, but is not limited to the genes for Bacillus subtilis alkaline protease (aprE), Bacillus subtilis neutral protease (nprT), Saccharomyces cerevisiae alpha-factor, Rhizomucor miehei aspartic proteinase, and Myceliophthora thermophila lactase (See e.g., WO 95/33836). Where both signal peptide and propeptide regions are present at the amino terminus of a polypeptide, the propeptide region is positioned next to the amino terminus of a polypeptide and the signal peptide region is positioned next to the amino terminus of the propeptide region.

In some embodiments, regulatory sequences are also utilized. These sequences facilitate the regulation of the expression of the polypeptide relative to the growth of the host cell. Examples of regulatory systems are those which cause the expression of the gene to be turned on or off in response to a chemical or physical stimulus, including the presence of a regulatory compound. In prokaryotic host cells, suitable regulatory sequences include, but are not limited to the lac, tac, and trp operator systems. In yeast host cells, suitable regulatory systems include, but are not limited to the ADH2 system or GAL1 system. In filamentous fungi, suitable regulatory sequences include, but are not limited to the TAKA alpha-amylase promoter, Aspergillus niger glucoamylase promoter, and Aspergillus oryzae glucoamylase promoter.

The present invention also provides recombinant expression vectors comprising a polynucleotide encoding an engineered monoamine oxidase polypeptide, and one or more expression regulating regions such as a promoter and a terminator, a replication origin, etc., depending on the type of hosts into which they are to be introduced. In some embodiments, the various nucleic acid and control sequences described above are combined together to produce a recombinant expression vector which includes one or more convenient restriction sites to allow for insertion or substitution of the nucleic acid sequence encoding the variant monoamine oxidase polypeptide at such sites. Alternatively, the polynucleotide sequence(s) of the present invention are expressed by inserting the polynucleotide sequence or a nucleic acid construct comprising the polynucleotide sequence into an appropriate vector for expression. In creating the expression vector, the coding sequence is located in the vector so that the coding sequence is operably linked with the appropriate control sequences for expression.

The recombinant expression vector may be any vector (e.g., a plasmid or virus), that can be conveniently subjected to recombinant DNA procedures and can result in the expression of the variant monoamine oxidase polynucleotide sequence. The choice of the vector will typically depend on the compatibility of the vector with the host cell into which the vector is to be introduced. The vectors may be linear or closed circular plasmids.

In some embodiments, the expression vector is an autonomously replicating vector (i.e., a vector that exists as an extra-chromosomal entity, the replication of which is independent of chromosomal replication, such as a plasmid, an extra-chromosomal element, a minichromosome, or an artificial chromosome). The vector may contain any means for assuring self-replication. In some alternative embodiments, the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell, or a transposon may be used.

In some embodiments, the expression vector preferably contains one or more selectable markers, which permit easy selection of transformed cells. A “selectable marker” is a gene the product of which provides for biocide or viral resistance, resistance to heavy metals, prototrophy to auxotrophy, and the like. Examples of bacterial selectable markers include, but are not limited to the dal genes from Bacillus subtilis or Bacillus licheniformis, or markers, which confer antibiotic resistance such as ampicillin, kanamycin, chloramphenicol or tetracycline resistance. Suitable markers for yeast host cells include, but are not limited to ADE2, HIS3, LEU2, LYS2, MET3, TRP1, and URA3. Selectable markers for use in a filamentous fungal host cell include, but are not limited to, amdS (acetamidase), argB (ornithine carbamoyltransferases), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. In another aspect, the present invention provides a host cell comprising a polynucleotide encoding at least one engineered monoamine oxidase of the present invention, the polynucleotide being operatively linked to one or more control sequences for expression of the engineered monoamine oxidase enzyme(s) in the host cell. Host cells for use in expressing the polypeptides encoded by the expression vectors of the present invention are well known in the art and include but are not limited to, bacterial cells, such as E. coli, Vibrio fluvialis, Streptomyces and Salmonella typhimurium cells; fungal cells, such as yeast cells (e.g., Saccharomyces cerevisiae and Pichia pastoris [ATCC Accession No. 201178]); insect cells such as Drosophila S2 and Spodoptera Sf9 cells; animal cells such as CHO, COS, BHK, 293, and Bowes melanoma cells; and plant cells. Exemplary host cells are Escherichia coli strains (e.g., W3110 (ΔfhuA) and BL21).

Accordingly, in another aspect, the present invention provides methods for producing the engineered monoamine oxidase polypeptides, where the methods comprise culturing a host cell capable of expressing a polynucleotide encoding the engineered monoamine oxidase polypeptide under conditions suitable for expression of the polypeptide. In some embodiments, the methods further comprise the steps of isolating and/or purifying the monoamine oxidase polypeptides, as described herein.

Appropriate culture media and growth conditions for the above-described host cells are well known in the art. Polynucleotides for expression of the monoamine oxidase polypeptides may be introduced into cells by various methods known in the art. Techniques include, among others, electroporation, biolistic particle bombardment, liposome mediated transfection, calcium chloride transfection, and protoplast fusion.

The engineered monoamine oxidases with the properties disclosed herein can be obtained by subjecting the polynucleotide encoding the naturally occurring or engineered monoamine oxidase polypeptide to mutagenesis and/or directed evolution methods known in the art, and as described herein. An exemplary directed evolution technique is mutagenesis and/or DNA shuffling (See e.g., Stemmer, Proc. Natl. Acad. Sci. USA 91:10747-10751 [1994]; WO 95/22625; WO 97/0078; WO 97/35966; WO 98/27230; WO 00/42651; WO 01/75767 and U.S. Pat. No. 6,537,746). Other directed evolution procedures that can be used include, among others, staggered extension process (StEP), in vitro recombination (See e.g., Zhao et al., Nat. Biotechnol., 16:258-261 [1998]), mutagenic PCR (See e.g., Caldwell et al., PCR Methods Appl., 3:S136-S140 [1994]), and cassette mutagenesis (See e.g., Black et al., Proc. Natl. Acad. Sci. USA 93:3525-3529 [1996]).

For example, mutagenesis and directed evolution methods can be readily applied to polynucleotides to generate variant libraries that can be expressed, screened, and assayed. Mutagenesis and directed evolution methods are well known in the art (See e.g., U.S. Pat. Nos. 5,605,793, 5,811,238, 5,830,721, 5,834,252, 5,837,458, 5,928,905, 6,096,548, 6,117,679, 6,132,970, 6,165,793, 6,180,406, 6,251,674, 6,265,201, 6,277,638, 6,287,861, 6,287,862, 6,291,242, 6,297,053, 6,303,344, 6,309,883, 6,319,713, 6,319,714, 6,323,030, 6,326,204, 6,335,160, 6,335,198, 6,344,356, 6,352,859, 6,355,484, 6,358,740, 6,358,742, 6,365,377, 6,365,408, 6,368,861, 6,372,497, 6,337,186, 6,376,246, 6,379,964, 6,387,702, 6,391,552, 6,391,640, 6,395,547, 6,406,855, 6,406,910, 6,413,745, 6,413,774, 6,420,175, 6,423,542, 6,426,224, 6,436,675, 6,444,468, 6,455,253, 6,479,652, 6,482,647, 6,483,011, 6,484,105, 6,489,146, 6,500,617, 6,500,639, 6,506,602, 6,506,603, 6,518,065, 6,519,065, 6,521,453, 6,528,311, 6,537,746, 6,573,098, 6,576,467, 6,579,678, 6,586,182, 6,602,986, 6,605,430, 6,613,514, 6,653,072, 6,686,515, 6,703,240, 6,716,631, 6,825,001, 6,902,922, 6,917,882, 6,946,296, 6,961,664, 6,995,017, 7,024,312, 7,058,515, 7,105,297, 7,148,054, 7,220,566, 7,288,375, 7,384,387, 7,421,347, 7,430,477, 7,462,469, 7,534,564, 7,620,500, 7,620,502, 7,629,170, 7,702,464, 7,747,391, 7,747,393, 7,751,986, 7,776,598, 7,783,428, 7,795,030, 7,853,410, 7,868,138, 7,783,428, 7,873,477, 7,873,499, 7,904,249, 7,957,912, 7,981,614, 8,014,961, 8,029,988, 8,048,674, 8,058,001, 8,076,138, 8,108,150, 8,170,806, 8,224,580, 8,377,681, 8,383,346, 8,457,903, 8,504,498, 8,589,085, 8,762,066, 8,768,871, 9,593,326, and all related US, as well as PCT and non-US counterparts; Ling et al., Anal. Biochem., 254(2):157-78 [1997]; Dale et al., Meth. Mol. Biol., 57:369-74 [1996]; Smith, Ann. Rev. Genet., 19:423-462 [1985]; Botstein et al., Science, 229:1193-1201 [1985]; Carter, Biochem. J., 237:1-7 [1986]; Kramer et al., Cell, 38:879-887 [1984]; Wells et al., Gene, 34:315-323 [1985]; Minshull et al., Curr. Op. Chem. Biol., 3:284-290 [1999]; Christians et al., Nat. Biotechnol., 17:259-264 [1999]; Crameri et al., Nature, 391:288-291 [1998]; Crameri, et al., Nat. Biotechnol., 15:436-438 [1997]; Zhang et al., Proc. Nat. Acad. Sci. U.S.A., 94:4504-4509 [1997]; Crameri et al., Nat. Biotechnol., 14:315-319 [1996]; Stemmer, Nature, 370:389-391 [1994]; Stemmer, Proc. Nat. Acad. Sci. USA, 91:10747-10751 [1994]; WO 95/22625; WO 97/0078; WO 97/35966; WO 98/27230; WO 00/42651; WO 01/75767; and WO 2009/152336, all of which are incorporated herein by reference).

In some embodiments, the enzyme clones obtained following mutagenesis treatment are screened by subjecting the enzymes to a defined temperature (or other assay conditions, such as testing the enzyme's activity over a broad range of substrates) and measuring the amount of enzyme activity remaining after heat treatments or other assay conditions. Clones containing a polynucleotide encoding a monoamine oxidase polypeptide are then sequenced to identify the nucleotide sequence changes (if any), and used to express the enzyme in a host cell. Measuring enzyme activity from the expression libraries can be performed using any suitable method known in the art (e.g., standard biochemistry techniques, such as HPLC analysis).

In some embodiments, the clones obtained following mutagenesis treatment can be screened for engineered monoamine oxidases having one or more desired improved enzyme properties (e.g., improved regioselectivity). Measuring enzyme activity from the expression libraries can be performed using the standard biochemistry techniques, such as GC analysis, HPLC analysis and/or derivatization of products (pre or post separation), for example, using dansyl chloride or OPA (See e.g., Yaegaki et al., J Chromatogr. 356(1):163-70 [1986]).

When the sequence of the engineered polypeptide is known, the polynucleotides encoding the enzyme can be prepared by standard solid-phase methods, according to known synthetic methods. In some embodiments, fragments of up to about 100 bases can be individually synthesized, then joined (e.g., by enzymatic or chemical ligation methods, or polymerase mediated methods) to form any desired continuous sequence. For example, polynucleotides and oligonucleotides encoding portions of the monoamine oxidase can be prepared by chemical synthesis as known in the art (e.g., the classical phosphoramidite method of Beaucage et al., Tet. Lett. 22:1859-69 [1981], or the method described by Matthes et al., EMBO J. 3:801-05 [1984]) as typically practiced in automated synthetic methods.

According to the phosphoramidite method, oligonucleotides are synthesized (e.g., in an automatic DNA synthesizer), purified, annealed, ligated and cloned in appropriate vectors. In addition, essentially any nucleic acid can be obtained from any of a variety of commercial sources. In some embodiments, additional variations can be created by synthesizing oligonucleotides containing deletions, insertions, and/or substitutions, and combining the oligonucleotides in various permutations to create engineered monoamine oxidases with improved properties.

Accordingly, in some embodiments, a method for preparing the engineered monoamine oxidase polypeptide comprises: (a) synthesizing a polynucleotide encoding a polypeptide comprising an amino acid sequence having at least about 80%, 85%, 86%, 87%, 88%, 89%, 90%, 91%, 92%, 93%, 94%, 95%, 96%, 97%, 98%, or 99% or more sequence identity to an amino acid sequence selected from the even-numbered sequences of SEQ ID NOs: 6-1084, and having one or more residue differences as compared to SEQ ID NO: 6 or 414; and (b) expressing the monoamine oxidase polypeptide encoded by the polynucleotide.

In some embodiments of the method, the polynucleotide encodes an engineered monoamine oxidase that has optionally one or several (e.g., up to 3, 4, 5, or up to 10) amino acid residue deletions, insertions and/or substitutions. In some embodiments, the amino acid sequence has optionally 1-2, 1-3, 1-4, 1-5, 1-6, 1-7, 1-8, 1-9, 1-10, 1-15, 1-20, 1-21, 1-22, 1-23, 1-24, 1-25, 1-30, 1-35, 1-40, 1-45, or 1-50 amino acid residue deletions, insertions and/or substitutions. In some embodiments, the amino acid sequence has optionally 1, 2, 3, 4, 5, 6, 7, 8, 9, 10, 11, 12, 13, 14, 15, 16, 17, 18, 19, 20, 21, 22, 23, 24, 25, 30, 30, 35, 40, 45, or 50 amino acid residue deletions, insertions and/or substitutions. In some embodiments, the amino acid sequence has optionally 1, 2, 3, 4, 5, 6, 7, 8, 9, 10, 11, 12, 13, 14, 15, 16, 18, 20, 21, 22, 23, 24, or 25 amino acid residue deletions, insertions and/or substitutions. In some embodiments, the substitutions can be conservative or non-conservative substitutions.

In some embodiments, any of the engineered monoamine oxidase enzymes expressed in a host cell can be recovered from the cells and/or the culture medium using any one or more of the well-known techniques for protein purification, including, among others, lysozyme treatment, sonication, filtration, salting-out, ultra-centrifugation, and chromatography. Suitable solutions for lysing and the high efficiency extraction of proteins from bacteria, such as E. coli, are commercially available (e.g., CelLytic B™, Sigma-Aldrich, St. Louis MO).

Chromatographic techniques for isolation of the monoamine oxidase polypeptide include, among others, reverse phase chromatography high performance liquid chromatography, ion exchange chromatography, gel electrophoresis, and affinity chromatography. Conditions for purifying a particular enzyme will depend, in part, on factors such as net charge, hydrophobicity, hydrophilicity, molecular weight, molecular shape, etc., and will be apparent to those having skill in the art.

In some embodiments, affinity techniques may be used to isolate the improved monoamine oxidase enzymes. For affinity chromatography purification, any antibody which specifically binds the monoamine oxidase polypeptide may be used. For the production of antibodies, various host animals, including but not limited to rabbits, mice, rats, etc., may be immunized by injection with a monoamine oxidase polypeptide, or a fragment thereof. The monoamine oxidase polypeptide or fragment may be attached to a suitable carrier, such as BSA, by means of a side chain functional group or linkers attached to a side chain functional group. In some embodiments, the affinity purification can use a specific ligand bound by the monoamine oxidase or dye affinity column (See e.g., EP0641862; Stellwagen, “Dye Affinity Chromatography,” In Current Protocols in Protein Science, Unit 9.2-9.2.16 [2001]).

### Methods of Using the Monoamine Oxidase Enzymes

In some embodiments, the monoamine oxidase enzymes described herein find use in processes for conversion of one or more suitable substrates to a product. In some embodiments, the monoamine oxidase enzymes described herein find use in processes for conversion of one or more suitable amine substrates to a one or more imine products. In some embodiments, the imine product is a fused bicyclic proline analogue.

In another aspect, the engineered monoamine oxidase polypeptides disclosed herein can be used in a process for the conversion of the substrate of Formula 1 to the product of Formula II, as depicted above in Scheme 1.

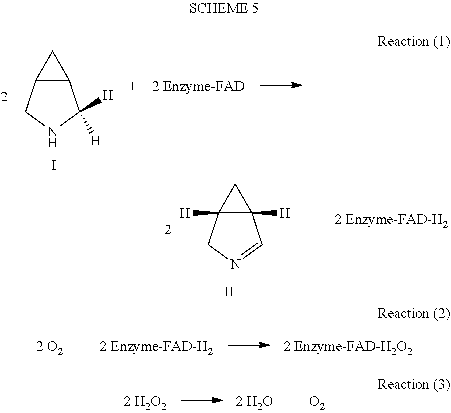

Scheme 5 depicts the three elementary reactions that, together, provide the overall net reaction depicted in Scheme 1. In the first reaction of Scheme 5, the secondary amine, a heterobicyclic compound according to structural Formula I is enantioselectively oxidized to the corresponding imine compound of structural Formula II by an engineered monoamine oxidase of the disclosure (which is complexed with a flavin adenine nucleotide co-factor (FAD)) to provide the corresponding substantially enantiomerically pure imine of structural Formula II and the reduced monoamine oxidase FAD complex (MAO-FAD-H₂).

In the second step, the reduced monoamine oxidase (MAO-FAD-H₂ complex) is re-oxidized by molecular oxygen, yielding hydrogen peroxide (H₂O₂) as a byproduct. In the third reaction (which is not catalyzed by the engineered monoamine oxidase), hydrogen peroxide (H₂O₂) breaks down to water and oxygen.

In certain embodiments, the present disclosure provides engineered monoamine oxidases capable of converting amine compounds of Formula I to imine compounds of Formula II. In one embodiment, the engineered monoamine oxidase polypeptides are capable of catalyzing the conversion of 6,6-dimethyl-3-azabicyclo[3.1.0]hexane (Compound 1) to (1R,5S)-6,6-dimethyl-3-azabicyclo[3.1.0]hex-2-ene (Compound 2), as depicted below in Scheme 6.

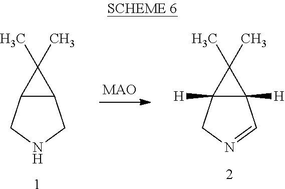

In some embodiments, the present disclosure provides engineered monoamine oxidase enzymes having improved activity in the production of Compound 2 as compared to a reference enzyme. In some embodiments, the engineered monoamine oxidase enzymes have the activity of Scheme 6.

In some embodiments, the monoamine oxidase enzymes described herein find use in processes for conversion of one or more suitable substrates to a product.

In another aspect, the present disclosure provides a process for the conversion of the substrate of Formula I to the product of Formula II using one or more engineered monoamine oxidases. In one embodiment, the present disclosure provides a process for the conversion of the substrate of Compound 1 to the product of Compound 2 using one or more engineered monoamine oxidases.

In some embodiments, the present disclosure provides a process of preparing a product of Formula II;

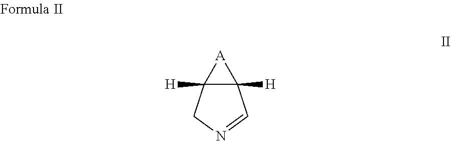

the process comprising a step of contacting a substrate of Formula I

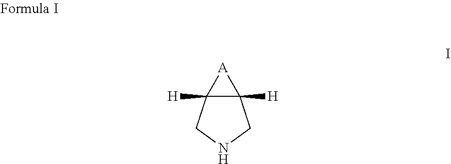

with an engineered monoamine oxidase polypeptide, as disclosed herein under suitable reaction conditions, such that a product of Formula II is prepared.

In some embodiments, the present disclosure provides a process of preparing Compound 2;

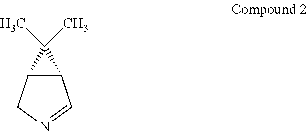

the process comprising a step of contacting a substrate of Compound 1

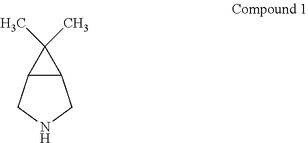

with an engineered monoamine oxidase polypeptide, as disclosed herein under suitable reaction conditions, such that a product of Compound 2 is prepared.

In some embodiments, the process of preparing Compound 2 from Compound 1 comprises a step of contacting Compound 1 with an engineered monoamine oxidase comprising 80%, 85%, 86%, 87%, 88%, 89%, 90%, 91%, 92%, 93%, 94%, 95%, 96%, 97%, 98%, or 99% or more sequence identity to a polypeptide sequence selected from the even-numbered sequences of SEQ ID NOs: 6-1084. In some embodiments, the process of preparing Compound 2 from Compound 1 comprises a step of contacting Compound 1 with an engineered monoamine oxidase comprising a polypeptide sequence selected from the even-numbered sequences of SEQ ID NOs: 6-1084.

In certain embodiments, the present disclosure provides engineered monoamine oxidases capable of converting amine compounds of Formula I to imine compounds of Formula II, which may be further converted to the aminosulfonate products of Formula III, as depicted above in Scheme 2. In another embodiment, the aminosulfonate product of Formula III is further converted to an amino acid in the presence of an acid and water.

In one embodiment, Compound 2 is converted to Compound 3 to sequester the oxidation product, as depicted below in Scheme 7. Compound 3 is then quenched with NaOH to isolate and purify the product of Compound 2.

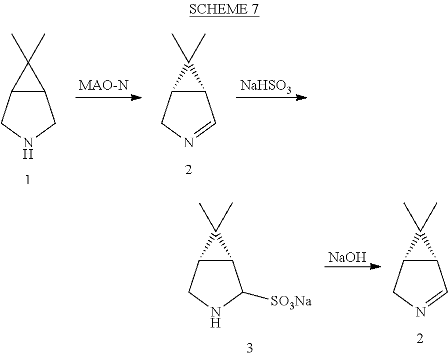

In the embodiments provided herein and illustrated in the Examples, various ranges of suitable reaction conditions that can be used in the processes, include but are not limited to, substrate loading, co-factor loading, pH, temperature, buffer, solvent system, polypeptide loading, oxygen sparging, antifoam, and reaction time. Further suitable reaction conditions for carrying out the process for biocatalytic conversion of substrate compounds to product compounds using an engineered monoamine described herein can be readily optimized in view of the guidance provided herein by routine experimentation that includes, but is not limited to, contacting the engineered monoamine polypeptide and one or more substrate compounds under experimental reaction conditions of concentration, pH, temperature, and solvent conditions, and detecting the product compound.

Substrate secondary amines of structural Formula I are commercially available or are readily synthesized using methods and reagents either known in the art or readily adapted, in light of the present disclosure, from methods and reagents known in the art (see e.g. Delalu et al. (1999) J. Heterocyclic Chem. 36, 681; WO 2007/022459; and WO 2007/075790, and references cited therein).

Hydrogen peroxide is a strong oxidizing agent capable of irreversibly inactivating the monoamine oxidase enzyme. Accordingly, in certain embodiments, a component useful for facilitating the disproportionation reaction depicted in step 3 of Scheme 5, above, in which hydrogen peroxide (H₂O₂) is broken down to molecular oxygen and water. In certain embodiments, that component is selected from among chemical agents, such as but not limited to Pd, Fe, and the like, while in other embodiments, that component is an enzyme, such as the enzyme catalase. In a particular embodiment, the reaction mixture further comprises the enzyme catalase to catalyze the disproportionation reaction of step 3 of Scheme 5, in which two molecules of hydrogen peroxide are broken down to provide two molecules of water and one of molecular oxygen. In particular embodiments, the catalase is an Aspergillus niger catalase that is included in the reaction at a concentration of from about 0.01% to about 1% (w/v), from about 0.05% to about 0.5% (w/v), or from about 0.1% to about 0.2% (w/v). In certain other embodiments, the catalase is a bovine catalase.

In those instances in which the compound of structural Formula I is volatile liquid, to facilitate handling, it may be provided as a salt. In one aspect of this embodiment, the amine substrate is converted to an acetate salt by addition of one equivalent of acetic acid to a 10% solution of the free base dissolved, e.g., in heptane. The precipitated salt is collected, washed with solvent (e.g., the solvent from which the salt is precipitated, e.g., heptane) and dried at room temperature (about 21° C.) under reduced pressure.

As indicated in Scheme 1, the ultimate oxidant is molecular oxygen. In view of the limited solubility of oxygen in water and in light of the decrease in that solubility as the temperature and salinity (solute concentration) increase, molecular oxygen in solution for reaction must be replenished by gas-liquid mass transfer from a gas phase. Typically, the activity and amount of preferred monoamine oxidases provided for practical reaction rates are sufficient for the reaction to become limited by the rate of gas-liquid mass transfer. As is well known, the rate of gas-liquid mass transfer is dependent on the partial pressure of the gas in the liquid, the solubility of the gas in the liquid, and the gas-liquid interfacial area. According, the rate of a gas-liquid mass transfer rate-limited reaction can be increased by engineering environments that provide aggressive gas-liquid mixing, including by sparging, hollow shaft impeller aspiration, countercurrent gas-liquid circulation, vertically serpentine tubular flow, and the like.

Additionally, in any engineering environment, the rate of an oxygen gas-liquid mass transfer rate-limited reaction can be increased by increasing the partial pressure of oxygen, either by increasing the total gas pressure, increasing the fraction of oxygen in the gas (such as enriching or replacing air with purified oxygen, of both. In particular embodiments, the concentration of dissolved oxygen and/or the rate of oxygen consumption from the gas phase is continuously monitored and the oxygen feed rate, partial pressure, mixing efficiency, or combinations thereof are adjusted to provide a beneficial rate of reaction or overall reaction time to completion.

The substrate compound(s) in the reaction mixtures can be varied, taking into consideration, for example, the desired amount of product compound, the effect of each substrate concentration on enzyme activity, stability of enzyme under reaction conditions, and the percent conversion of each substrate to product. In some embodiments, the suitable reaction conditions comprise a substrate compound loading of at least about 0.5 to about 25 g/L, 1 to about 25 g/L, 5 to about 25 g/L, about 10 to about 25 g/L, or 20 to about 25 g/L. In some embodiments, the suitable reaction conditions comprise a substrate compound loading of at least about 0.5 g/L, at least about 1 g/L, at least about 3 g/L, at least about 5 g/L, at least about 10 g/L, at least about 15 g/L, at least about 20 g/L, or at least about 30 g/L, or even greater.

In carrying out the monoamine oxidase mediated processes described herein, the engineered polypeptide may be added to the reaction mixture in the form of a purified enzyme, partially purified enzyme, whole cells transformed with gene(s) encoding the enzyme, as cell extracts and/or lysates of such cells, and/or as an enzyme immobilized on a solid support. Whole cells transformed with gene(s) encoding the engineered monoamine oxidase enzyme or cell extracts, lysates thereof, and isolated enzymes may be employed in a variety of different forms, including solid (e.g., lyophilized, spray-dried, and the like) or semisolid (e.g., a crude paste). The cell extracts or cell lysates may be partially purified by precipitation (ammonium sulfate, polyethyleneimine, heat treatment or the like, followed by a desalting procedure prior to lyophilization (e.g., ultrafiltration, dialysis, etc.). Any of the enzyme preparations (including whole cell preparations) may be stabilized by crosslinking using known crosslinking agents, such as, for example, glutaraldehyde or immobilization to a solid phase (e.g., Eupergit C, and the like).

The gene(s) encoding the monoamine oxidase polypeptides can be transformed into host cell separately or together into the same host cell. For example, in some embodiments one set of host cells can be transformed with gene(s) encoding one engineered monoamine oxidase polypeptide and another set can be transformed with gene(s) encoding another engineered monoamine oxidase polypeptide. Both sets of transformed cells can be utilized together in the reaction mixture in the form of whole cells, or in the form of lysates or extracts derived therefrom. In other embodiments, a host cell can be transformed with gene(s) encoding multiple engineered monoamine oxidase polypeptides. In some embodiments the engineered polypeptides can be expressed in the form of secreted polypeptides, and the culture medium containing the secreted polypeptides can be used for the monoamine oxidase reaction.

In some embodiments, the improved activity of the engineered monoamine oxidase polypeptides disclosed herein provides for processes wherein higher percentage conversion can be achieved with lower concentrations of the engineered polypeptide. In some embodiments of the process, the suitable reaction conditions comprise an engineered polypeptide amount of about 0.5% (w/w), 1% (w/w), 1.5% (w/w), 2% (w/w), 3% (w/w), 4% (w/w), 5% (w/w), 10% (w/w), 20% (w/w), 30% (w/w), 40% (w/w), 50% (w/w), 75% (w/w), 100% (w/w) or more of substrate compound loading.

In some embodiments, the engineered polypeptide is present at about 0.01 g/L to about 50 g/L; about 0.05 g/L to about 50 g/L; about 0.1 g/L to about 40 g/L; about 1 g/L to about 40 g/L; about 2 g/L to about 40 g/L; about 5 g/L to about 40 g/L; about 5 g/L to about 30 g/L; about 0.1 g/L to about 10 g/L; about 0.5 g/L to about 10 g/L; about 1 g/L to about 10 g/L; about 0.1 g/L to about 5 g/L; about 0.5 g/L to about 5 g/L; or about 0.1 g/L to about 2 g/L. In some embodiments, the monoamine oxidase polypeptide is present at about 0.01 g/L, 0.05 g/L, 0.1 g/L, 0.2 g/L, 0.5 g/L, 1, 2 g/L, 5 g/L, 10 g/L, 15 g/L, 20 g/L, 25 g/L, 30 g/L, 35 g/L, 40 g/L, or 50 g/L.

During the course of the reaction, the pH of the reaction mixture may change. The pH of the reaction mixture may be maintained at a desired pH or within a desired pH range. This may be done by the addition of an acid or a base (such as NaOH), before and/or during the course of the reaction.

Alternatively, the pH may be controlled by using a buffer. Accordingly, in some embodiments, the reaction condition comprises a buffer. Suitable buffers to maintain desired pH ranges are known in the art and include, by way of example and not limitation, sodium phosphate, borate, phosphate, 2-(N-morpholino)ethanesulfonic acid (MES), 3-(N-morpholino)propanesulfonic acid (MOPS), acetate, triethanolamine, and 2-amino-2-hydroxymethyl-propane-1,3-diol (Tris), and the like. In some embodiments, the reaction conditions comprise water as a suitable solvent with no buffer present.

In the embodiments of the process, the reaction conditions comprise a suitable pH. The desired pH or desired pH range can be maintained by use of an acid or base (such as NaOH), an appropriate buffer, or a combination of buffering and acid or base addition. The pH of the reaction mixture can be controlled before and/or during the course of the reaction. In some embodiments, the suitable reaction conditions comprise a solution pH from about 4 to about 11, pH from about 5 to about 10, pH from about 5 to about 9, pH from about 6 to about 9, pH from about 6 to about 8. In some embodiments, the reaction conditions comprise a solution pH of about 4, 4.5, 5, 5.5, 6, 6.5, 7, 7.5, 8, 8.5, 9, 9.5, 10, or 11.

In the embodiments of the processes herein, a suitable temperature is used for the reaction conditions, for example, taking into consideration the increase in reaction rate at higher temperatures, and the activity of the enzyme during the reaction time period. Accordingly, in some embodiments, the suitable reaction conditions comprise a temperature of about 10° C. to about 60° C., about 10° C. to about 55° C., about 15° C. to about 60° C., about 20° C. to about 60° C., about 20° C. to about 55° C., about 25° C. to about 55° C., or about 30° C. to about 50° C. In some embodiments, the suitable reaction conditions comprise a temperature of about 10° C., 15° C., 20° C., 25° C., 30° C., 35° C., 40° C., 45° C., 50° C., 55° C., or 60° C.

In some embodiments, the temperature during the enzymatic reaction can be maintained at a specific temperature throughout the course of the reaction. In some embodiments, the temperature during the enzymatic reaction can be adjusted over a temperature profile during the course of the reaction.

In some embodiments, the processes of the invention are carried out in a solvent. Suitable solvents include water, aqueous buffer solutions, organic solvents, polymeric solvents, and/or co-solvent systems, which generally comprise aqueous solvents, organic solvents and/or polymeric solvents. The aqueous solvent (water or aqueous co-solvent system) may be pH-buffered or unbuffered. In some embodiments, the processes using the engineered monoamine oxidase polypeptides can be carried out in an aqueous co-solvent system comprising an organic solvent (e.g., ethanol, isopropanol (IPA), dimethyl sulfoxide (DMSO), dimethylformamide (DMF) ethyl acetate, butyl acetate, 1-octanol, heptane, octane, methyl tert-butyl ether (MTBE), toluene, and the like), ionic or polar solvents (e.g., 1-ethyl-4-methylimidazolium tetrafluoroborate, 1-butyl-3-methylimidazolium tetrafluoroborate, 1-butyl-3-methylimidazolium hexafluorophosphate, glycerol, polyethylene glycol, and the like). In some embodiments, the co-solvent can be a polar solvent, such as a polyol, dimethylsulfoxide (DMSO), or lower alcohol. The non-aqueous co-solvent component of an aqueous co-solvent system may be miscible with the aqueous component, providing a single liquid phase, or may be partly miscible or immiscible with the aqueous component, providing two liquid phases. Exemplary aqueous co-solvent systems can comprise water and one or more co-solvents selected from an organic solvent, polar solvent, and polyol solvent. In general, the co-solvent component of an aqueous co-solvent system is chosen such that it does not adversely inactivate the monoamine oxidase enzyme under the reaction conditions. Appropriate co-solvent systems can be readily identified by measuring the enzymatic activity of the specified engineered monoamine oxidase enzyme with a defined substrate of interest in the candidate solvent system, utilizing an enzyme activity assay, such as those described herein.

In some embodiments of the process, the suitable reaction conditions comprise an aqueous co-solvent, where the co-solvent comprises DMSO at about 1% to about 50% (v/v), about 1 to about 40% (v/v), about 2% to about 40% (v/v), about 5% to about 30% (v/v), about 10% to about 30% (v/v), or about 10% to about 20% (v/v). In some embodiments of the process, the suitable reaction conditions can comprise an aqueous co-solvent comprising ethanol at about 1% (v/v), about 5% (v/v), about 10% (v/v), about 15% (v/v), about 20% (v/v), about 25% (v/v), about 30% (v/v), about 35% (v/v), about 40% (v/v), about 45% (v/v), or about 50% (v/v).

In some embodiments, the reaction conditions comprise a surfactant for stabilizing or enhancing the reaction. Surfactants can comprise non-ionic, cationic, anionic and/or amphiphilic surfactants. Exemplary surfactants, include by way of example and not limitation, nonyl phenoxypolyethoxylethanol (NP40), TRITON™ X-100 polyethylene glycol tert-octylphenyl ether, polyoxyethylene-stearylamine, cetyltrimethylammonium bromide, sodium oleylamidosulfate, polyoxyethylene-sorbitanmonostearate, hexadecyldimethylamine, etc. Any surfactant that may stabilize or enhance the reaction may be employed. The concentration of the surfactant to be employed in the reaction may be generally from 0.1 to 50 mg/ml, particularly from 1 to 20 mg/ml.

In some embodiments, the reaction conditions include an antifoam agent, which aids in reducing or preventing formation of foam in the reaction solution, such as when the reaction solutions are mixed or sparged. Antifoam agents include non-polar oils (e.g., minerals, silicones, etc.), polar oils (e.g., fatty acids, alkyl amines, alkyl amides, alkyl sulfates, etc.), and hydrophobic (e.g., treated silica, polypropylene, etc.), some of which also function as surfactants. Exemplary antifoam agents include, Antifoam-204 or Antifoam Y-30 (Sigma, St. Louis MO), Y-30® (Dow Corning), poly-glycol copolymers, oxy/ethoxylated alcohols, and polydimethylsiloxanes. In some embodiments, the antifoam can be present at about 0.001% (v/v) to about 5% (v/v), about 0.01% (v/v) to about 5% (v/v), about 0.1% (v/v) to about 5% (v/v), or about 0.1% (v/v) to about 2% (v/v). In some embodiments, the antifoam agent can be present at about 0.001% (v/v), about 0.01% (v/v), about 0.1% (v/v), about 0.5% (v/v), about 1% (v/v), about 2% (v/v), about 3% (v/v), about 4% (v/v), or about 5% (v/v) or more as desirable to promote the reaction.

The quantities of reactants used in the monoamine oxidase reaction will generally vary depending on the quantities of product desired, and concomitantly the amount of monoamine oxidase substrate employed. Those having ordinary skill in the art will readily understand how to vary these quantities to tailor them to the desired level of productivity and scale of production.

In some embodiments, the order of addition of reactants is not critical. The reactants may be added together at the same time to a solvent (e.g., monophasic solvent, biphasic aqueous co-solvent system, and the like), or alternatively, some of the reactants may be added separately, and some together at different time points. For example, the cofactor and substrate may be added first to the solvent.

The solid reactants (e.g., enzyme, salts, etc.) may be provided to the reaction in a variety of different forms, including powder (e.g., lyophilized, spray dried, and the like), solution, emulsion, suspension, and the like. The reactants can be readily lyophilized or spray dried using methods and equipment that are known to those having ordinary skill in the art. For example, the protein solution can be frozen at −80° C. in small aliquots, then added to a pre-chilled lyophilization chamber, followed by the application of a vacuum.

For improved mixing efficiency when an aqueous co-solvent system is used, the monoamine oxidase, and co-factor may be added and mixed into the aqueous phase first. The monoamine oxidase substrate may be added and mixed in, followed by the organic phase or the substrate may be dissolved in the organic phase and mixed in. Alternatively, the monoamine oxidase substrate may be premixed in the organic phase, prior to addition to the aqueous phase.

The processes of the present invention are generally allowed to proceed until further conversion of substrate to product does not change significantly with reaction time (e.g., less than 10% of substrate being converted, or less than 5% of substrate being converted). In some embodiments, the reaction is allowed to proceed until there is complete or near complete conversion of substrate to product. Transformation of substrate to product can be monitored using known methods by detecting substrate and/or product, with or without derivatization. Suitable analytical methods include gas chromatography, HPLC, MS, and the like.

In some embodiments of the process, the suitable reaction conditions comprise a substrate loading for each of one or more substrates of at least about 5 g/L, 10 g/L, 20 g/L, 30 g/L, 40 g/L, 50 g/L, 60 g/L, 70 g/L, 80 g/L, 90 g/L, 100 g/L, or more, and wherein the method results in at least about 50%, 60%, 70%, 80%, 90%, 95% or greater conversion of substrate compound to product compound in about in about 24 h or less, in about 12 h or less, in about 6 h or less, or in about 4 h or less.

In some further embodiments of the processes for converting one or more substrate compounds to product compound using the engineered monoamine oxidase polypeptides, the suitable reaction conditions can comprise an initial substrate loading to the reaction solution which is then contacted by the polypeptide. This reaction solution is then further supplemented with additional substrate compound as a continuous or batchwise addition over time at a rate of at least about 3 g/L/h, at least about 5 g/L/h, at least about 8 g/L/h, at least about 10 g/L/h, or higher for the substrate compound. Thus, according to these suitable reaction conditions, polypeptide is added to a solution having an initial substrate loading of at least about 10 g/L, 20 g/L, or 30 g/L. This addition of polypeptide is then followed by continuous addition of further substrate to the solution at a rate of about 3 g/L/h, at least about 5 g/L/h, at least about 8 g/L/h, at least about 10 g/L/h, or higher until a much higher final substrate loading of at least about 60 g/L or more is reached. Accordingly, in some embodiments of the process, the suitable reaction conditions comprise addition of the polypeptide to a solution having an initial substrate loading of at least about 10 g/L, 20 g/L, or 30 g/L followed by addition of further substrate to the solution at a rate of 3 g/L/h, at least about 5 g/L/h, at least about 8 g/L/h, at least about 10 g/L/h, or higher until a final substrate loading of at least about 60 g/L, or more, is. This substrate supplementation reaction condition allows for higher substrate loadings to be achieved while maintaining high rates of conversion of substrate to product of at least about 5%, 25%, 50%, 75%, 90% or greater conversion of substrate.

In some embodiments of the process, the suitable reaction conditions comprise a cofactor. In some embodiments, the cofactor is selected from the group consisting of FAD, FMN, NADP, and NAD. In a particular embodiment, the co-factor is FAD. In some embodiments, the suitable reaction conditions comprise an FDA co-factor at about 0.005 mM, 0.01 mM, 0.05 mM, 0.1 mM, 0.15 mM, 0.2 mM, 0.3 mM, or 0.4 mM.

In some embodiments of the process, the suitable reaction conditions comprise a component useful for facilitating the disproportionation of the hydrogen peroxide side product of the monoamine oxidase catalyzed reaction to molecular oxygen and water. In certain embodiments, that component is Pd, Fe, or catalase. In a particular embodiment, the suitable reaction conditions comprise catalase. In some embodiments, the suitable reaction conditions comprise catalase at about 0.0005 g/L, 0.001 g/L, 0.01 g/L, 0.1 g/L, 0.5 g/L, or 1 g/L.

In certain embodiments of the processes disclosed herein, the suitable reaction conditions comprise a monoamine oxidase enzyme with a co-factor and with bisulfite under conditions which yield a mixture comprising an aminosulfonate. In some embodiments, the bisulfite is added to the reaction before or simultaneously with the monoamine oxidase. In some embodiments, the bisulfate is added after addition of the monoamine oxidase. In certain other embodiments, the amine substrate is contacted with oxygen and the monoamine oxidase enzyme associated with a co-factor before addition of the bisulfite. In certain further embodiments, the suitable reaction conditions further comprise cyanide, such that the aminosulfonate is further converted to an aminonitrile. In yet further embodiments, the aminonitrile is further converted to an amino acid in the presence of an acid and water.

Any of the processes disclosed herein using the engineered polypeptides for the preparation of compounds of Formula II, including but not limited to Compound 2, can be carried out under a range of suitable reaction conditions, including but not limited to ranges of substrates, ranges FAD cofactors, ranges of catalase, temperature, pH, solvent system, polypeptide loading, and reaction time. In one example, in some embodiments, the preparation of compounds of Formula II, including but not limited to Compound 2, can be carried out wherein the suitable reaction conditions comprise: (a) substrate loading of about 0.0001 M to 1.0 M of substrate compound; (b) FAD co-factor loading of about 0.005 mM to 0.3 mM; (c) loading of about 0.001 g/L to 100 g/L of engineered polypeptide; (d) 0.01 M-1 M sodium phosphate buffer; (e) 0.0005 to 1 g/L catalase (or equivalent, as described herein); (f) pH at 6-9; (g) non-rate limiting O₂; and (g) temperature of about 20° C. to 60° C. In some embodiments, the suitable reaction conditions comprise: (a) about 20 mM of Compound 1 substrate; (b) FAD co-factor of about 0.01 mM; (c) about 0.004 g/L engineered polypeptide; (d) 50 mM sodium phosphate buffer; (e) 0.02 g/L of bovine catalase; (f) pH at 7.5; (g) non-rate limiting O₂; and (g) temperature of about 25° C.

In some embodiments, an engineered monoamine oxidase polypeptide capable of converting Compound 1 to Compound 2 comprises an amino acid sequence that is at least about 85%, 86%, 87%, 88%, 89%, 90%, 91%, 92%, 93%, 94%, 95%, 96%, 97%, 98%, or 99% identical to a reference sequence of SEQ ID NO: 6 or 414, wherein the polypeptide comprises an amino acid sequence where the amino acid residue corresponding to residue position 429 is glycine, wherein the residues are numbered with reference to the amino acid sequence of SEQ ID NO: 6 or 414, and wherein the engineered monoamine oxidase polypeptide has 2-fold, 5-fold, or 10-fold or more the activity of the engineered monoamine oxidase polypeptide of SEQ ID NO: 6 or 414.

In some embodiments, an engineered monoamine oxidase polypeptide capable of converting Compound 1 to Compound 2 comprises an amino acid sequence that is at least about 85%, 86%, 87%, 88%, 89%, 90%, 91%, 92%, 93%, 94%, 95%, 96%, 97%, 98%, or 99% identical to a reference sequence of SEQ ID NO: 6 or 414, wherein the polypeptide comprises an amino acid sequence where the amino acid residue corresponding to residue position 347 is cysteine or asparagine, and the amino acid residue corresponding to residue position 429 is glycine, wherein the residues are numbered with reference to the amino acid sequence of SEQ ID NO: 6 or 414, and wherein the engineered monoamine oxidase polypeptide has 2-fold, 5-fold, or 10-fold or more the activity of the engineered monoamine oxidase polypeptide of SEQ ID NO: 6 or 414.

In some embodiments, an engineered monoamine oxidase polypeptide capable of converting Compound 1 to Compound 2 comprises an amino acid sequence that is at least about 85%, 86%, 87%, 88%, 89%, 90%, 91%, 92%, 93%, 94%, 95%, 96%, 97%, 98%, or 99% identical to a reference sequence of SEQ ID NO: 6 or 414, wherein the polypeptide comprises an amino acid sequence where the amino acid residue corresponding to residue position 347 is cysteine or asparagine, the amino acid residue corresponding to residue position 421 is cysteine, and the amino acid residue corresponding to residue position 429 is glycine, wherein the residues are numbered with reference to the amino acid sequence of SEQ ID NO: 6 or 414, and wherein the engineered monoamine oxidase polypeptide has 2-fold, 5-fold, or 10-fold or more the activity of the engineered monoamine oxidase polypeptide of SEQ ID NO: 6 or 414.

In some embodiments, an engineered monoamine oxidase polypeptide capable of converting Compound 1 to Compound 2 comprises an amino acid sequence that is at least about 85%, 86%, 87%, 88%, 89%, 90%, 91%, 92%, 93%, 94%, 95%, 96%, 97%, 98%, or 99% identical to a reference sequence of SEQ ID NO: 6 or 414, wherein the polypeptide comprises an amino acid sequence where the amino acid residue corresponding to residue position 145 is leucine, the amino acid residue corresponding to residue position 149 is methionine, the amino acid residue corresponding to residue position 198 is aspartic acid, the amino acid residue corresponding to residue position 260 is serine, the amino acid residue corresponding to residue position 266 is leucine, the amino acid residue corresponding to residue position 338 is glycine, the amino acid residue corresponding to residue position 347 is asparagine, and the amino acid residue corresponding to residue position 429 is glycine, wherein the residues are numbered with reference to the amino acid sequence of SEQ ID NO: 6 or 414, and wherein the engineered monoamine oxidase polypeptide has 2-fold, 5-fold, or 10-fold or more the activity of the engineered monoamine oxidase polypeptide of SEQ ID NO: 6 or 414.

In some embodiments, an engineered monoamine oxidase polypeptide capable of converting Compound 1 to Compound 2 comprises an amino acid sequence that is at least about 85%, 86%, 87%, 88%, 89%, 90%, 91%, 92%, 93%, 94%, 95%, 96%, 97%, 98%, or 99% identical to a reference sequence of SEQ ID NO: 6 or 414, wherein the polypeptide comprises an amino acid sequence where the amino acid residue corresponding to residue position 87 is leucine, the amino acid residue corresponding to residue position 113 is glycine, the amino acid residue corresponding to residue position 117 is lysine, the amino acid residue corresponding to residue position 260 is serine, the amino acid residue corresponding to residue position 338 is serine, and the amino acid residue corresponding to residue position 421 is serine, wherein the residues are numbered with reference to the amino acid sequence of SEQ ID NO: 6 or 414, and wherein the engineered monoamine oxidase polypeptide has 2-fold, 5-fold, or 10-fold or more the activity of the engineered monoamine oxidase polypeptide of SEQ ID NO: 6 or 414.

In some embodiments, additional reaction components or additional techniques carried out to supplement the reaction conditions. These can include taking measures to stabilize or prevent inactivation of the enzyme, reduce product inhibition, shift reaction equilibrium to formation of the desired product.

In further embodiments, any of the above described process for the conversion of one or more substrate compounds to product compound can further comprise one or more steps selected from: extraction; isolation; purification; and crystallization of product compound. Methods, techniques, and protocols for extracting, isolating, purifying, and/or crystallizing the product from biocatalytic reaction mixtures produced by the above disclosed processes are known to the ordinary artisan and/or accessed through routine experimentation. Additionally, illustrative methods are provided in the Examples below.

Various features and embodiments of the invention are illustrated in the following representative examples, which are intended to be illustrative, and not limiting.

### EXPERIMENTAL

The following Examples, including experiments and results achieved, are provided for illustrative purposes only and are not to be construed as limiting the present invention.

In the Examples below, the following abbreviations apply: ppm (parts per million); M (molar); mM (millimolar), uM and μM (micromolar); nM (nanomolar); mol (moles); gm and g (gram); mg (milligrams); ug and μg (micrograms); L and l (liter); ml and mL (milliliter); cm (centimeters); mm (millimeters); um and μm (micrometers); sec. (seconds); min(s) (minute(s)); h(s) and hr(s) (hour(s)); U (units); MW (molecular weight); rpm (rotations per minute); psi and PSI (pounds per square inch); ° C. (degrees Centigrade); RT and rt (room temperature); CAM and cam (chloramphenicol); DMSO (dimethylsulfoxide); PMBS (polymyxin B sulfate); IPTG (isopropyl β-D-1-thiogalactopyranoside); LB (Luria-Bertani broth); TB (Terrific Broth; 12 g/L bacto-tryptone, 24 g/L yeast extract, 4 mL/L glycerol, 65 mM potassium phosphate, pH 7.0, 1 mM MgSO₄); PLP (pyridoxal 5′-phosphate), TEoA (triethanolamine buffer), HEPES (HEPES zwitterionic buffer; 4-(2-hydroxyethyl)-piperazineethanesulfonic acid); SFP (shake flask powder); CDS (coding sequence); DNA (deoxyribonucleic acid); RNA (ribonucleic acid); E. coli W3110 (commonly used laboratory E. coli strain, available from the Coli Genetic Stock Center [CGSC], New Haven, CT); HTP (high throughput); HPLC (high pressure liquid chromatography); FIOPC (fold improvements over positive control); Microfluidics (Microfluidics, Corp., Westwood, MA); Sigma-Aldrich (Sigma-Aldrich, St. Louis, MO; Difco (Difco Laboratories, BD Diagnostic Systems, Detroit, MI); Agilent (Agilent Technologies, Inc., Santa Clara, CA); Corning (Corning, Inc., Palo Alto, CA); Dow Corning (Dow Corning, Corp., Midland, MI); and Gene Oracle (Gene Oracle, Inc., Mountain View, CA).

### Example 1

### Preparation of HTP MAO-Containing Wet Cell Pellets

E. coli cells containing recombinant MAO-encoding genes from monoclonal colonies were inoculated into 180 μl LB containing 1% glucose and 30 μg/mL chloramphenicol in the wells of 96-well shallow-well microtiter plates. The plates were sealed with O₂-permeable seals, and cultures were grown overnight at 30° C., 200 rpm, and 85% humidity. Then, 10 μl of each of the cell cultures were transferred into the wells of 96-well deep-well plates containing 390 mL TB and 30 μg/mL CAM. The deep-well plates were sealed with O₂-permeable seals and incubated at 30° C., 250 rpm, and 85% humidity until OD₆₀₀ 0.6-0.8 was reached. The cell cultures were then induced by IPTG to a final concentration of 1 mM and incubated overnight at 30° C. The cells were then pelleted using centrifugation at 4,000 rpm for 10 min. The supernatants were discarded, and the pellets frozen at −80° C. prior to lysis.

### Example 2

### Preparation of HTP MAO-Containing Wet Cell Lysates

Frozen pellets prepared as described in Example 2 were lysed with 400 μl lysis buffer containing 100 mM sodium phosphate buffer, pH 7.0. 1 g/L lysozyme and 0.5 g/L polymyxin b sulfate (PMBS) was added to the cell paste in each well. The cells were lysed at room temperature for 2 hours with shaking on a bench top shaker. The plate was then centrifuged for 10 min at 4,000 rpm at 4° C. The clear supernatants were then used in biocatalytic reactions to determine their activity levels.

### Example 3

### Preparation of Lyophilized Monoamine Oxidase Lysates from Shake Flask (SF) Cultures

A single colony containing the desired gene was picked from an LB agar plate with 1% glucose and 30 μg/ml CAM, and grown overnight at 37° C. A single colony from each culture was transferred to 6 ml of LB with 1% glucose and 30 μg/ml CAM. The cultures were grown for 18 h at 30° C. and 250 rpm and subcultured approximately 1:50 into 250 ml of TB containing 30 μg/ml CAM, to a final OD₆₀₀ of 0.05. The cultures were grown for approximately 195 minutes at 30° C. and 250 rpm to an OD₆₀₀ between 0.6-0.8 and induced with 1 mM IPTG. The cultures were then grown for 20 h at 30° C. and 250 rpm. The cultures were centrifuged 4,000 rpm for 20 min. The supernatant was discarded, and the pellets were resuspended in 30 ml of 50 mM sodium phosphate, pH 7.0, and lysed using a Microfluidizer® processor system (Microfluidics) at 18,000 psi. The lysates were pelleted (10,000 rpm for 60 min), and the supernatants were frozen and lyophilized to generate shake flake (SF) enzyme powders.

### Example 4

### Improvements in Activity in MAO Relative to SEQ ID NO: 6 in High Throughput Screening

SEQ ID NO: 6 was selected as the parent enzyme that was already evolved towards improved amine monooxygenase activity and stability and disclosed in WO2010/008828A2. SEQ ID NO: 6 contains the following mutations compared to the wild-type enzyme of SEQ ID NO: 2: A289V/T315S/I316V/Q317H/A321P/T324P/E325Q/I327M/S328A/M330A/Q331N/A332I/M337Q/T339 V/D346S/N347C/K348P/T354S/A357S/C364A/L379I/F382L/T384G/D385A/A386H/D392E/R396E/E39 7A/L399K/K400M/G403E/Q404N/L405M/A406S/T409N/F410M/G411D/V412I/V421C/S433A/R434P/G436Q/M437L/V438L/E440K/C441S/Q443D/G444E/E447C/K448R/R450G/G451N/V453L/S465G/R47 9V/V480T/L482I/G486R/T487P/K488A/R489P/E490A/K492R/A493S/R494H. Libraries of engineered genes were produced using well established techniques (e.g., saturation mutagenesis, recombination of previously identified beneficial mutations). The polypeptides encoded by each gene were produced in HTP, and cell pellets were obtained, and the soluble lysate was generated as described in Example 2.

HTP reactions were performed in a 96-well format in 2 mL deep-well plates, with 100 μL total volume. HTP reactions contain 3 g/L (18 mM) 6,6-dimethyl-3-azabicyclo[3.1.0]hexane acetate, 4U catalase, 50 mM sodium phosphate pH 7.4, and 4,000× diluted MAO-N lysate. The reactions were set up as follows: (i) all the reaction components, except for catalase and MAO-N, were mixed in a single solution and pH adjusted to 7.4 using H₃PO₄. Catalase was added, and the resultant solution (90 μL) was then aliquoted into each well of the 96-well plates and (ii) diluted MAO-N lysate (10 μL of 400x) was then added into the wells to initiate the reaction. The reaction plate was heat-sealed with a foil seal and incubated at 30° C. with 600 rpm shaking for 16-18 hours. The reactions were quenched with 100 μL of a 1:1 mixture of acetonitrile in 20 mM ammonium acetate. The quenched reactions were shaken for 3 min on a tabletop shaker followed by centrifugation at 4,000 rpm for 10 mins at 4° C. to pellet any precipitate. Supernatant (90 μL) was then transferred into a 96-well round bottom plate. The samples were analyzed according to the RP-HPLC analytical method in Table 4.1.

TABLE 4.1
RP-HPLC Analytical Method

| | |
|---|---|
| Instrument | ThermoScientific U3000 UPLC with UV |
| | Detection |
| Column | Zorbax Eclipse C18 2.1 mm × 75 mm, 3.5 μm |
| Mobile Phase | Isocratic 75:25 40 mM |
| | ammonium acetate:acetonitrile |
| Flow Rate | 0.5 mL/min |
| Run Time | 2.0 min |
| Product Elution | 1.4 min |
| Column Temperature | 40° C. |
| Injection Volume | 5 μL |
| Detection | UV 254 nm |
| | Detector: Thermo VWD-3400; Peak width 0.02 |
| | min; Collection rate = 200 Hz; Time Constant = |
| | 0.12 s |

Activity relative to SEQ ID NO: 6 was calculated as fold improvement over positive control (FIOPC). It was determined by dividing the integrated area under the curve of the imine signal in each sample by area under the curve of the imine signal for parent variant (positive control) present in the same plate under the specified reaction conditions and was shown in Table 4.2.

TABLE 4.2
HTP MAO Activity for
the Production of Compound (2) Relative to SEQ ID NO: 6

| | | Product Area |
|---|---|---|
| SEQ | | Fold Improvement |
| ID NO: | Amino Acid Differences | (Relative to |
| (nt/aa) | (Relative to SEQ ID NO: 6) | SEQ ID NO: 6)¹ |
| 7/8 | W79D | +++ |
| 9/10 | S80V | +++ |
| 11/12 | P87G | +++ |
| 13/14 | W79Q | +++ |
| 15/16 | P87L | +++ |
| 17/18 | W79S | +++ |
| 19/20 | A429G | +++ |
| 21/22 | P87S | +++ |
| 23/24 | L219P/I332L | +++ |
| 25/26 | N82F | +++ |
| 27/28 | P87Y | +++ |
| 29/30 | S80I | +++ |
| 31/32 | W79A | +++ |
| 33/34 | P87W | +++ |
| 35/36 | R260S | +++ |
| 37/38 | S80H | +++ |
| 39/40 | S80N | +++ |
| 41/42 | F250V | +++ |
| 43/44 | P87H | +++ |
| 45/46 | D184F | +++ |
| 47/48 | D146A | +++ |
| 49/50 | D184R | +++ |
| 51/52 | Y238K | +++ |
| 53/54 | N82R | +++ |
| 55/56 | I332G | +++ |
| 57/58 | N82W | +++ |
| 59/60 | Y238R | +++ |
| 61/62 | D146F | +++ |
| 63/64 | L149M | +++ |
| 65/66 | C347N | +++ |
| 67/68 | T93G | +++ |
| 69/70 | C338G | +++ |
| 71/72 | H418G | +++ |
| 73/74 | S187P/M242I | +++ |
| 75/76 | L310S | +++ |
| 77/78 | W262F/A266L/C421S | +++ |
| 79/80 | C338S | +++ |
| 81/82 | S80T | +++ |
| 83/84 | C338A | +++ |
| 85/86 | P117K | +++ |
| 87/88 | D252G | ++ |
| 89/90 | E145S | ++ |
| 91/92 | N113G | ++ |
| 93/94 | E145G | ++ |
| 95/96 | P117R | ++ |
| 97/98 | L219S | ++ |
| 99/100 | I332L | ++ |
| 101/102 | T218G | ++ |
| 103/104 | W262F/C421S/C447A | ++ |
| 105/106 | M242I | ++ |
| 107/108 | L445R | ++ |
| 109/110 | H386E | ++ |
| 111/112 | R181P | ++ |
| 113/114 | C421S | ++ |
| 115/116 | F425L | ++ |
| 117/118 | L199T | ++ |
| 119/120 | L115M | ++ |
| 121/122 | F170K | ++ |
| 123/124 | I332S | ++ |
| 125/126 | E145L | ++ |
| 127/128 | M174G | ++ |
| 129/130 | L153T | ++ |
| 131/132 | N221C | ++ |
| 133/134 | V177R | ++ |
| 135/136 | E198D | ++ |
| 137/138 | P87Q/E185F | ++ |
| 139/140 | S234V | ++ |
| 141/142 | H386G | ++ |
| 143/144 | K413R | ++ |
| 145/146 | E32G/N284D | ++ |
| 147/148 | E393G | ++ |
| 149/150 | C381A | ++ |
| 151/152 | D252L | ++ |
| 153/154 | V167C | ++ |
| 155/156 | D252E | ++ |
| 157/158 | C447A | ++ |
| 159/160 | G240A | ++ |
| 161/162 | E185K | ++ |
| 163/164 | H229E | ++ |
| 165/166 | E147A | ++ |
| 167/168 | A433N | ++ |
| 169/170 | G161R | ++ |
| 171/172 | T162Q | ++ |
| 173/174 | T55A/C347D/P348K | ++ |
| 175/176 | L149M/T157A | ++ |
| 177/178 | N221V | ++ |
| 179/180 | S100P/A144E/C421S/C447A | ++ |
| 181/182 | E444L | ++ |
| 183/184 | E444D | ++ |
| 185/186 | E393K | ++ |
| 187/188 | T475I | ++ |
| 189/190 | K155L | ++ |
| 191/192 | I246M | ++ |
| 193/194 | M174T | ++ |
| 195/196 | R260F | ++ |
| 197/198 | E392G | ++ |
| 199/200 | E32G/T65V/T135Q/I246M/N284D | ++ |
| 201/202 | E185G | ++ |
| 203/204 | E208S | ++ |
| 205/206 | G47A/D197N | ++ |
| 207/208 | P117I | ++ |
| 209/210 | N387Q | + |
| 211/212 | H449R | + |
| 213/214 | T107W | + |
| 215/216 | R181C | + |
| 217/218 | W79V | + |
| 219/220 | E483R | + |
| 221/222 | D193A | + |
| 223/224 | P117C | + |
| 225/226 | A433S | + |
| 227/228 | A144E/C447A | + |
| 229/230 | N387W | + |
| 231/232 | R196G | + |
| 233/234 | R196M | + |
| 235/236 | P359S | + |
| 237/238 | M174V | + |
| 239/240 | L149V | + |
| 241/242 | S441H | + |
| 243/244 | T65V/T135Q | + |
| 245/246 | K155C | + |
| 247/248 | C306V | + |
| 249/250 | R260V | + |
| 251/252 | A300R | + |
| 253/254 | V177G | + |
| 255/256 | K155R | + |
| 257/258 | T475E | + |
| 259/260 | H127D | + |
| 261/262 | N82L | + |
| 263/264 | N221R | + |
| 265/266 | K155Q | + |
| 267/268 | E392S | + |
| 269/270 | S323R | + |
| 271/272 | E185L | + |
| 273/274 | E393M | + |
| 275/276 | A266L/C447A | + |
| 277/278 | N113W | + |
| 279/280 | P117A | + |
| 281/282 | D411V/L445R | + |
| 283/284 | T13G | + |
| 285/286 | L199V | + |
| 287/288 | E298C | + |
| 289/290 | N361L | + |
| 291/292 | T166G | + |
| 293/294 | G161L | + |
| 295/296 | N387I | + |
| 297/298 | S100P | + |
| 299/300 | M400D | + |
| 301/302 | P407G | + |
| 303/304 | Q239G | + |
| 305/306 | A114G | + |
| 307/308 | T65V/N284D | + |
| 309/310 | E32G/T135Q/N284D | + |
| 311/312 | G85S | + |
| 313/314 | W79Y | + |
| 315/316 | T475L | + |
| 317/318 | A366G | + |
| 319/320 | S100P/A144E/C447A | + |
| 321/322 | V452M | + |
| 323/324 | T136R | + |
| 325/326 | P117S | + |
| 327/328 | P117L | + |
| 329/330 | R181L | + |
| 331/332 | P169G | + |
| 333/334 | V177W | + |
| 335/336 | A385G | + |
| 337/338 | L445D | + |
| 339/340 | R290Y | + |
| 341/342 | P117T | + |
| 343/344 | A232T | + |
| 345/346 | T166L | + |
| 347/348 | C347D | + |
| 349/350 | H127R | + |
| 351/352 | E444S | + |
| 353/354 | T475S | + |
| 355/356 | G161D | + |
| 357/358 | L199I | + |
| 359/360 | G85D | + |
| 361/362 | R181G | + |
| 363/364 | M174S | + |
| 365/366 | G161Y | + |
| 367/368 | A144M | + |
| 369/370 | C277T | + |
| 371/372 | N284D | + |
| 373/374 | A61C | + |
| 375/376 | R294V | + |
| 377/378 | E185R | + |
| 379/380 | D349E | + |
| 381/382 | C277T/C347D | + |
| 383/384 | T65V/I246M | + |
| 385/386 | H449S | + |
| 387/388 | R104K | + |
| 389/390 | P434E | + |
| 391/392 | I211G | + |
| 393/394 | T65V/E190D | + |
| 395/396 | T292N | + |
| 397/398 | H127G | + |
| 399/400 | H418A | + |
| 401/402 | E444R | + |
| 403/404 | R290Q | + |
| 405/406 | T135G | + |
| 407/408 | Q254A | + |
| 409/410 | T166E | + |
| 411/412 | D252A | + |

Levels of increased activity were determined relative to the reference polypeptide of SEQ ID NO: 6 and defined as follows: “+” 1.00 to 1.09, “++” >1.09, “+++” >1.30.

### Example 5

### Improvements in Activity in MAO Relative to SEQ ID NO: 6 in Shake Flask Validation

Reactions were performed in a 96-well format in 2 mL deep-well plates, with 100 μL total volume. Reactions included 3 g/L (18 mM) 6,6-dimethyl-3-azabicyclo[3.1.0]hexane acetate, 4U catalase, 50 mM sodium phosphate pH 7.4, and 0.01 g/L MAO-N SFP. The reactions were set up as follows: (i) all the reaction components, except for catalase and MAO-N, were mixed in a single solution and pH adjusted to 7.4 using H₃PO₄. Catalase was added, and the resultant solution (90 μL) was then aliquoted into each well of the 96-well plates, and (ii) diluted MAO-N SFP (10 μL of 0.1 μL) was then added into the wells to initiate the reaction. The reaction plate was heat-sealed with a foil seal and incubated at 30° C. with 600 rpm shaking for 16-18 hours. The reactions were quenched with 100 μL of a 1:1 mixture of acetonitrile in 20 mM ammonium acetate. The quenched reactions were shaken for 3 min on a tabletop shaker followed by centrifugation at 4,000 rpm for 10 mins at 4° C. to pellet any precipitate. Supernatant (90 μL) was then transferred into a 96-well round bottom plate. The samples were analyzed according to the RP-HPLC analytical method in Table 4.1.

TABLE 5.1
MAO Activity for the Production of Compound (2) Relative to
SEQ ID NO: 6

| | | Product Area Fold |
|---|---|---|
| SEQ ID | | Improvement |
| NO: | Amino Acid Differences | (Relative to |
| (nt/aa) | (Relative to SEQ ID NO: 6) | SEQ ID NO: 6)¹ |
| 413/414 | A429G | +++ |
| 11/12 | P87G | +++ |
| 77/78 | W262F/A266L/C421S | ++ |
| 63/64 | L149M | ++ |
| 69/70 | C338G | ++ |
| 7/8 | W79D | + |
| 35/36 | R260S | + |
| 59/60 | Y238R | + |
| 9/10 | S80V | + |
| 23/24 | L219P/I332L | + |
| 47/48 | D146A | + |

Levels of increased activity were determined relative to the reference polypeptide of SEQ ID NO: 6 and defined as follows: ″+″ .90 to 1.35, ″++″ > 1.35, ″+++″ > 2.08

### Example 6

### Improvements in Activity in MAO Relative to SEQ ID NO: 6 in High Throughput Screening

HTP reactions were performed in a 96-well format in 2 mL deep-well plates, with 100 μL total volume. HTP reactions contain 3 g/L (18 mMv) 6,6-dimethyl-3-azabicyclo[3.1.0]hexane acetate, 4U catalase, 50 mM sodium phosphate pH 7.4, and 4,000× diluted MAO-N lysate. The reactions were set up as follows: (i) all the reaction components, except for catalase and MAO-N, were mixed in a single solution and pH adjusted to 7.4 using H₃PO₄. Catalase was added, and the resultant solution (90 μL) was then aliquoted into each well of the 96-well plates, and (ii) diluted MAO-N lysate (10 μL of 400×) was then added into the wells to initiate the reaction. The reaction plate was heat-sealed with a foil seal and incubated at 30° C. with 600 rpm shaking for 16-18 hours. The reactions were quenched with 100 μL of a 1:1 mixture of acetonitrile in 20 mMv ammonium acetate. The quenched reactions were shaken for 3 min on a tabletop shaker followed by centrifugation at 4,000 rpm for 10 mins at 4° C. to pellet any precipitate. Supernatant (90 μL) was then transferred into a 96-well round bottom plate. The samples were analyzed according to the RP-HPLC analytical method in Table 4.1.

TABLE 6.1
HTP MAO Activity for the Production of Compound (2)
Relative to SEQ ID NO: 6

| | | Product |
|---|---|---|
| | | Area Fold |
| | | Improvement |
| SEQ ID | | (Relative to |
| NO: | Amino Acid Differences | SEQ ID |
| (nt/aa) | (Relative to SEQ ID NO: 6) | NO: 6)¹ |
| 415/416 | P87L/C421S | +++ |
| 417/418 | N82R/P87G | +++ |
| 419/420 | N82F/P87G/T218G/C338S | +++ |
| 421/422 | P87G/T218G/L219S/C338S | +++ |
| 423/424 | N82R/R260S/C338A/C421S | +++ |
| 425/426 | N82F/P87L | +++ |
| 427/428 | P87G/R260S | +++ |
| 429/430 | N82F/P87L/L219S/C421S | +++ |
| 431/432 | N82F/P87L/N113G/P117K/T218G | +++ |
| 433/434 | P87L/T218G/H418G | +++ |
| 435/436 | P87L/N113G/P117K/R260S/C338S/ | +++ |
| | C421S | |
| 437/438 | R260S/C338G | +++ |
| 439/440 | K110N/C338G/H418G | +++ |
| 441/442 | N82R/N113G/C338S | +++ |
| 443/444 | L219S/C421S | +++ |
| 445/446 | N82F/P87G/N113G/P117K/R260S/ | +++ |
| | C338G | |
| 447/448 | S80H/A429G | +++ |
| 449/450 | N82F/P87G/P117K | +++ |
| 451/452 | P87L/P117K/T218G/L219S | +++ |
| 453/454 | W79D/Y183F/N284D/I332G | +++ |
| 455/456 | N82R/P87G/N113G/C421S | +++ |
| 457/458 | N82F/P87L/C338S | +++ |
| 459/460 | P117K/L219S | +++ |
| 461/462 | N82F/P87G/C338S/H418G | +++ |
| 463/464 | P87G/N113G | +++ |
| 465/466 | N82R/P87G/P117K/R260S/C338S/ | +++ |
| | C421S | |
| 467/468 | P87L/N113G/L219S/C421S | +++ |
| 469/470 | P87L/C338A | +++ |
| 471/472 | N82R/C338A/H418G/C421S | +++ |
| 473/474 | L310S/C338S/H418G | +++ |
| 475/476 | P87G/L310S/C338S | +++ |
| 477/478 | P87G/C338A | +++ |
| 479/480 | P87L/L219S/C338G | +++ |
| 481/482 | S80V/H386E/A429G | +++ |
| 483/484 | S80H/Y183F/N284D/I332G/C381A/ | +++ |
| | H386E/A429G | |
| 485/486 | T218G/R260S/C338A | +++ |
| 487/488 | P87L/P117K | +++ |
| 489/490 | Y238R/N284D/H386E/A429G | +++ |
| 491/492 | D146A/L149M/M242I/I246M/L445R | +++ |
| 493/494 | N82R/P87L/N113G | +++ |
| 495/496 | N82R/P87G/P117K/C338A/H418G | +++ |
| 497/498 | S100P/E145S/D146A/L149M/E198D/ | +++ |
| | M242I/F250V/D252G/C347N/P348K/ | |
| | C447A | |
| 499/500 | P87G/H418G | +++ |
| 501/502 | W79D/S80V/I332G/A429G | +++ |
| 503/504 | P87L | +++ |
| 505/506 | N82F/P87L/P117K | +++ |
| 507/508 | E145G/L149M/S205T/I246M/F250V/ | +++ |
| | D252G | |
| 509/510 | N82R/N113G/L219S/C338S | +++ |
| 511/512 | H386E/A429G | +++ |
| 513/514 | N82R/P87L/T218G/L219S/C338S | +++ |
| 515/516 | M242L/N284D/C381A/A429G | +++ |
| 517/518 | N82F/H418G | +++ |
| 519/520 | W79D | +++ |
| 521/522 | D184R/N284D/A429G | +++ |
| 523/524 | E145G/F250V/D252G/P348K | +++ |
| 525/526 | N82F/P87G/T218G | +++ |
| 527/528 | P87G/N113G/P117K/H418G/C421S | +++ |
| 529/530 | Y238R/A429G | +++ |
| 531/532 | S100P/D146A/F250V/C447A | ++ |
| 533/534 | P87L/L219S | ++ |
| 535/536 | E145S/D252G/P348K/C447A | ++ |
| 537/538 | T218G | ++ |
| 539/540 | P87L/L219S/H418G | ++ |
| 541/542 | N82R/P87G/P117K/T218G/C421S | ++ |
| 543/544 | P87G/N113G/R260S | ++ |
| 545/546 | S100P/D146A/L149M/E198D/M242I/ | ++ |
| | I246M/C277T/C347D/P348K/L445R/ | |
| | C447A | |
| 547/548 | I332G/H386E | ++ |
| 549/550 | P87G/L310S | ++ |
| 551/552 | N82F/P87G/N113G/P117K/L219S | ++ |
| 553/554 | Y238R/C381A/A429G | ++ |
| 555/556 | S100P/E143D/E145S/D146A/L149M/ | ++ |
| | E198D/M242I/C347N/P348K/L445R/ | |
| | C447A | |
| 557/558 | S100P/E145S/L149M/C347N/L445R/ | ++ |
| | C447A | |
| 559/560 | D146A/C277T/C347N/L445R/C447A | ++ |
| 561/562 | R260S | ++ |
| 563/564 | S100P/L149M/F250V/C347N/C447A | ++ |
| 565/566 | N82R/T218G/L219S/C421S | |
| 567/568 | E145S/I246M/D252G/C347D/C447A | ++ |
| 569/570 | S100P/D146A/C347D/P348K/L445R/ | ++ |
| | C447A | |
| 571/572 | S100P/E145G/L149M/M242I/L445R/ | ++ |
| | C447A | |
| 573/574 | L310S/H418G/C421S | ++ |
| 575/576 | Y238R/H386E | ++ |
| 577/578 | T93G/Y238R/N284D | ++ |
| 579/580 | A429G | ++ |
| 581/582 | I332G/A429G | ++ |
| 583/584 | D146A/L445R/C447A | ++ |
| 585/586 | S100P/E145G/L149M/M242I/I246M/ | ++ |
| | C347D/P348K/L445R/C447A | |
| 587/588 | D184R/N284D | ++ |
| 589/590 | Y238R/I332G/A429G | ++ |
| 591/592 | N113G/C338A/C421S | ++ |
| 593/594 | N113G/T218G/C338S/C421S | ++ |
| 595/596 | N284D/H386E/A429G | ++ |
| 597/598 | E145G/S255R | ++ |
| 599/600 | E145G/L149M/E198D/C347N/C447A | ++ |
| 601/602 | E145G/D146A/L149M/C277T/C347D | ++ |
| 603/604 | S80V/T237S/I332G | ++ |
| 605/606 | S80H/C381A | ++ |
| 607/608 | S100P/D146A/E198D/L445R | ++ |
| 609/610 | S80H/I332G/C381A/H386E | ++ |
| 611/612 | S80V/I332G | ++ |
| 613/614 | S100P/E145G/D146A/L149M/F250V/ | ++ |
| | C277T/C347N/P348K/C447A | |
| 615/616 | L219S/C338A/H418G | ++ |
| 617/618 | S100P/E145S/L149M/E198D/M242I/ | ++ |
| | C277T/C347N/P348K/C447A | |
| 619/620 | S100P/D146A/E198D/C347D/P348K/ | ++ |
| | C447A | |
| 621/622 | S100P/E145S/D146A/L149M/S205T/ | ++ |
| | C347D/L445R/C447A | |
| 623/624 | S100P/E145S/L149M/I246M/C347N/ | ++ |
| | L445R | |
| 625/626 | R260S/C338G/H418G/C421S | ++ |
| 627/628 | E145S/L149M/C347N/P348K/L445R | ++ |
| 629/630 | W79A/T93G/H386E | ++ |
| 631/632 | D146A/L149M/I246M/D252G/C447A | ++ |
| 633/634 | S100P/E145S/D146A/L149M/E198D | ++ |
| 635/636 | E145G/L149M/S205T/M242I/C347N/ | ++ |
| | P348K/L445R | |
| 637/638 | P87L/N113G/P117K/L219S/C338G/ | ++ |
| | H418G/C421S | |
| 639/640 | D146A/C277T/C347N/P348K | ++ |
| 641/642 | W79D/S80V | ++ |
| 643/644 | E145S/C347D/P348K/L445R/C447A/ | ++ |
| | V491A | |
| 645/646 | S100P/C347N/P348K/L445R | ++ |
| 647/648 | P87G | ++ |
| 649/650 | S80H/H386E | ++ |
| 651/652 | S100P/E145G/E198D/A300V | ++ |
| 653/654 | N82F/N113G/T218G/C338S/H418G | ++ |
| 655/656 | S100P/E145G/L149M/S205T | ++ |
| 657/658 | N82R/P87L/C338G/C421S | ++ |
| 659/660 | S100P/E143V/E145G/L149M/C347N/ | ++ |
| | C447A | |
| 661/662 | E145G/D146A/L149M/C347D/P348K | ++ |
| 663/664 | S100P/M242I/C347D/P348K/L445R/ | ++ |
| | C447A | |
| 665/666 | S100P/E145G/L149M/E198D/M242I/ | ++ |
| | I246M/L445R/C447A | |
| 667/668 | S100P/M242I/C347D/P348K/L445R | ++ |
| 669/670 | E145S/L149M/E198D/S205T/P348K | ++ |
| 671/672 | S100P/E145S/I246M/C347N/P348K/ | ++ |
| | L445R | |
| 673/674 | S80V/I332G/H386E | ++ |
| 675/676 | S80V/N284D | ++ |
| 677/678 | C338G/H418G | ++ |
| 679/680 | E145G/L149M/M242I/P348K | ++ |
| 681/682 | S100P/E198D/C347D/P348K/L445R | ++ |
| 683/684 | D146A/M242I | ++ |
| 685/686 | I332G | ++ |
| 687/688 | H418G | ++ |
| 689/690 | S100P/D146A/M242I/C277T/C447A | ++ |
| 691/692 | E198D/F250V | ++ |
| 693/694 | N113G/C338G | ++ |
| 695/696 | E145S/D146A/E198D/L445R/C447A | ++ |
| 697/698 | T93G/Y238R/H386E/A429G | ++ |
| 699/700 | E145G/L149M/I246M | ++ |
| 701/702 | S100P/E198D/C447A | ++ |
| 703/704 | N82R/N113G/T218G/C338A | ++ |
| 705/706 | D146A/C277T | + |
| 707/708 | S100P/E145S/L149M/I246M/D252G/ | + |
| | C277T/C347N/L445R/C447A | |
| 709/710 | S100P/E145S/C277T/C347N | + |
| 711/712 | S100P/E145S/D146A/L149M | + |
| 713/714 | E145S/L149M/C347N/C447A | + |
| 715/716 | L149M/C347N/P348K/L445R/C447A | + |
| 717/718 | E145S/L149M/E198D/I246M/C447A | + |
| 719/720 | D146A/1246M/C277T/P348K/L445R/ | + |
| | C447A | |
| 721/722 | S100P/E145S/D146A/L149M/M242I/ | + |
| | L445R | |
| 723/724 | E145S/D146A/L149M/S205T/P348K/ | + |
| | C447A | |
| 725/726 | S80H/Y238R/H386E/A429G | + |
| 727/728 | D146A/E198D/C277T/P348K/C447A | + |
| 729/730 | E145S/L149M/M242I | + |
| 731/732 | S100P/E198D/C347N/L445R | + |
| 733/734 | S100P/E198D/C277T/C347N/P348K | + |
| 735/736 | S100P/E145G/L149M/H154L/I246M/ | + |
| | C277T | |
| 737/738 | D184R | + |
| 739/740 | S100P/L149M/I246M/C447A | + |
| 741/742 | E145S/L149M/E198D/M242I/C277T/ | + |
| | C347D | |
| 743/744 | N82R/P87L | + |
| 745/746 | P117K/R260S | + |
| 747/748 | L149M/E198D/P348K | + |
| 749/750 | S100P/C277T/C347D/L445R/C447A | + |
| 751/752 | E145S/C277T/P348K/C447A | + |
| 753/754 | T93G/D184R/A429G | + |
| 755/756 | N82F/P87L/N113G/P117K/L219S/ | + |
| | H418G | |
| 757/758 | D146A/C277T/L445R/C447A | + |
| 759/760 | S100P/E198D/C347D/P348K | + |
| 761/762 | N284D/I332G | + |
| 763/764 | S100P/E145S/L149M/E198D/M242I/ | + |
| | I246M/C277T/P348K/C447A | |
| 765/766 | P87L/N113G | + |
| 767/768 | S100P/M242I/P348K/L445R | + |
| 769/770 | D146A/L149M/C277T/P348K/C447A | + |
| 771/772 | D146A/P348K | + |
| 773/774 | T93G/Y238R | + |
| 775/776 | E145S/I246M | + |
| 777/778 | D146A/E198D/I246M/C277T | + |
| 779/780 | S100P/E145S/L149M/M242I/I246M/ | + |
| | C277T/C347N/L445R | |
| 781/782 | S100P/E145S/S205T/C277T/P348K | + |
| 783/784 | S205T/C347D/P348K/L445R/C447A | + |
| 785/786 | E145S/L149M/I246M/C347D/L445R | + |
| 787/788 | E145S/L445R/C447A | + |
| 789/790 | S100P/L149M/I246M/C277T/P348K/ | + |
| | L445R | |
| 791/792 | P87G/L219S | + |
| 793/794 | E198D/I246M | + |
| 795/796 | P87L/T218G | + |
| 797/798 | L149M/E198D/C277T/C347N/P348K | + |
| 799/800 | E145S/L149M/S205T/C277T/L445R | + |
| 801/802 | C347N/P348K/L445R | + |
| 803/804 | E145S/L149M/E198D/M242I/I246M/ | + |
| | P348K | |
| 805/806 | S100P/E145G/P348K | + |
| 807/808 | W79A/S80H | + |
| 809/810 | S80V/Y238R/I332G | + |
| 811/812 | E145S/L149M/I246M/C447A | + |
| 813/814 | S205T/I246M/C347N | + |
| 815/816 | D146A/C347D/P348K/C447A | + |
| 817/818 | S100P/L149M/M242I/C347N/P348K/ | + |
| | C447A | |
| 819/820 | S100P/C347N/C447A | + |
| 821/822 | N82F/P87G/T218G/L219S/C421S | + |
| 823/824 | N82R/P87L/L219S | + |
| 825/826 | E145G/E198D/S205T/P348K | + |
| 827/828 | S80H | + |
| 829/830 | S100P/L149M/M242I/I246M/C277T/ | + |
| | C347N/P348K/L445R | |
| 831/832 | S80H/Y188F/E190D/I332G/H386E | + |
| 833/834 | P87L/T218G/L219S | + |
| 835/836 | L219S | + |
| 837/838 | E145G/D146A/C277T/C347N | + |
| 839/840 | S100P/E145S/L149M/C277T/P348K/ | + |
| | L445R | |
| 841/842 | E145S/D146A/L149M/E198D/C277T/ | + |
| | L445R/C447A | |
| 843/844 | E145S/D146A/L149M/F250V/C277T/ | + |
| | C347D/P348K/C447A | |
| 845/846 | H386E | + |
| 847/848 | S100P/C277T/P348K | + |
| 849/850 | D146A/L149M/S151C/I246M/P348K | + |
| 851/852 | S100P/E198D/S205T/P348K | + |
| 853/854 | W79D/T93G | + |
| 855/856 | Y238R | + |
| 857/858 | L149M | + |
| 859/860 | S80V | + |
| 861/862 | E145S/L149M/C447A | + |
| 863/864 | S100P | + |
| 865/866 | S100P/L149M/E198D | + |
| 867/868 | S100P/M242I/C277T/P348K | + |
| 869/870 | M242I | + |
| 871/872 | S80V/H386E | + |
| 873/874 | S100P/I246M/C277T/P348K/L445R | + |
| 875/876 | W79A/T93G/Y238R/H386E/A429G | + |
| 877/878 | W79A/Y238R/I332G/A429G | + |
| 879/880 | I246M | + |
| 881/882 | S80V/N284D/H386E | + |
| 883/884 | C338G | + |
| 885/886 | T93G/Y238R/H386E | + |
| 887/888 | S205T/D252G/C347D/L445R/C447A | + |
| 889/890 | W79A/Y238R/A429G | + |
| 891/892 | E145S/D146A/E198D/S205T/M242I/ | + |
| | C277T/P348K/C447A | |
| 893/894 | N284D | + |
| 895/896 | N82F/P87L/N113G/P117K/C338G/ | + |
| | H418G | |
| 897/898 | E198D/C277T/P348K/C447A | + |
| 899/900 | N284D/H386E | + |
| 901/902 | I246M/L445R/C447A | + |
| 903/904 | D184R/H386E | + |
| 905/906 | E145S/D146A/L149M/E198D/M242I/ | + |
| | D252G/C277T/C347N | |
| 907/908 | I246M/C277T/P348K/C447A | + |
| 909/910 | S100P/L445R/C447A | + |
| 911/912 | S100P/E145G/E198D/M242I/I246M/ | + |
| | P348K | |
| 913/914 | E145S/L149M/E198D/M242I/I246M/ | + |
| | C277T/C347N/C447A | |
| 915/916 | S100P/C277T/P348K/C447A | + |
| 917/918 | P87L/N113G/T218G/L219S/R260S/ | + |
| | C338A | |
| 919/920 | E198D/L445R | + |
| 921/922 | E145S/D146A/L149M/H154L/ | + |
| | I246M/C277T/C347N/P348K | |
| 923/924 | E145S/D146A/I246M/C347N/ | + |
| | P348K/C447A | |
| 925/926 | S100P/M242I/I246M/C277T | + |
| 927/928 | F121C/E198D/C347N/L445R/C447A | + |
| 929/930 | S100P/E145G/L149M/E198D/ | + |
| | D252G/P348K/L445R | |
| 931/932 | C277T/C347N/C447A | + |
| 933/934 | S205T | + |
| 935/936 | M242I/P348K | + |
| 937/938 | C447A | + |
| 939/940 | C347N/P348K/C447A | + |
| 941/942 | S100P/M242I/I246M/C347N | + |
| 943/944 | M242I/L445R/C447A | + |
| 945/946 | P87G/P117K/L219S/L310S/C338S | + |
| 947/948 | E145G/K155I/L445R/C447A | + |
| 949/950 | D146A/M242I/I246M/C277T/C347N/ | + |
| | P348K/L445R/C447A | |
| 951/952 | L149M/M242I/I246M/P348K/L445R | + |
| 953/954 | P348K/C447A | + |
| 955/956 | E198D/M242I/P348K | + |
| 957/958 | L149M/E198D/L445R | + |
| 959/960 | S100P/E145G/M242I/I246M/C277T/ | + |
| | L445R/C447A | |
| 961/962 | L149M/L445R | + |
| 963/964 | E145G/C277T/C347N/L445R | + |
| 965/966 | S100P/L149M/M242I/I246M/C277T/ | + |
| | C347N/P348K/C447A | |
| 967/968 | E145G/D146A/C347D/C447A | + |
| 969/970 | P348K | + |
| 971/972 | S100P/E145G/S205T/I246M/C277T/ | + |
| | P348K/L445R | |
| 973/974 | N82F | + |
| 975/976 | S100P/E145S/D146A/L149M/ | + |
| | H154L/P348K/L445R | |
| 977/978 | L149M/M242I/I246M | + |
| 979/980 | C277T/C347N/P348K/L445R | + |
| 981/982 | M242I/C277T/C347D/P348K | + |
| 983/984 | W79A/Y238R | + |
| 985/986 | E145S/D146A/I246M/C277T/C447A | + |
| 987/988 | P348K/L445R/C447A | + |
| 989/990 | Y238R/M242L/N284D | + |
| 991/992 | S100P/E145G/E198D/C277T/P348K/ | + |
| | L445R | |
| 993/994 | E145G/D146A/L445R/C447A | + |

Levels of increased activity were determined relative to the reference polypeptide of SEQ ID NO: 6 and defined as follows: ″+″ .95 to 1.50, ″++″ > 1.50, ″+++″ > 2.50

### Example 7

### Improvements in Activity in MAO Relative to SEQ ID NO: 6 in Shake Flask Validation

Reactions were performed in a 96-well format in 2 mL deep-well plates, with 100 μL total volume. Reactions included 3 g/L (18 mMv) 6,6-dimethyl-3-azabicyclo[3.1.0]hexane acetate, 4U catalase, 50 mM sodium phosphate pH 7.4, and 0.004 g/L MAO-N SFP. The reactions were set up as follows: (i) all the reaction components, except for catalase and MAO-N, were mixed in a single solution and pH adjusted to 7.4 using H₃PO₄. Catalase was added, and the resultant solution (90 μL) was then aliquoted into each well of the 96-well plates, and (ii) diluted MAO-N SFP (10 μL of 0.04 g/L) was then added into the wells to initiate the reaction. The reaction plate was heat-sealed with a foil seal and incubated at 30° C. with 600 rpm shaking for 16-18 hours. The reactions were quenched with 100 μL of a 1:1 mixture of acetonitrile in 20 mM ammonium acetate. The quenched reactions were shaken for 3 min on a tabletop shaker followed by centrifugation at 4,000 rpm for 10 mins at 4° C. to pellet any precipitate. Supernatant (90 μL) was then transferred into a 96-well round bottom plate. The samples were analyzed according to the RP-HPLC analytical method in Table 4.1.

TABLE 7.1
MAO Activity for the Production of Compound (2) Relative
to SEQ ID NO: 6

| | | Product |
|---|---|---|
| | | Area Fold |
| SEQ ID | | Improvement |
| NO: | Amino Acid Differences | (Relative to |
| (nt/aa) | (Relative to SEQ ID NO: 6) | SEQ ID NO: 6)¹ |
| 435/436 | P87L/N113G/P117K/R260S/C338S/C421S | +++ |
| 415/416 | P87L/C421S | +++ |
| 995/996 | S80H/A429G | ++ |
| 439/440 | K110N/C338G/H418G | ++ |
| 507/508 | E145G/L149M/S205T/I246M/F250V/D252G | ++ |
| 515/516 | M242L/N284D/C381A/A429G | + |
| 497/498 | S100P/E145S/D146A/L149M/E198D/M242I/ | + |
| | F250V/D252G/C347N/P348K/C447A | |
| 617/618 | S100P/E145S/L149M/E198D/M242I/C277T/ | + |
| | C347N/P348K/C447A | |
| 997/998 | Y238R/H386E/A429G | + |
| 567/568 | E145S/I246M/D252G/C347D/C447A | + |
| 535/536 | E145S/D252G/P348K/C447A | + |

Levels of increased activity were determined relative to the reference polypeptide of SEQ ID NO: 6 and defined as follows: ″+″ 1.02 to 1.50, ″++″ > 1.50, ″+++″ > 1.8

### Example 8

### Improvements in Activity in MAO Relative to SEQ ID NO: 414 in High Throughput

Reactions were performed in a 96-well format in 2 mL deep-well plates, with 100 μL total volume. Reactions included 12.7 g/L (75 m) 6,6-dimethyl-3-azabicyclo[3.1.0]hexane acetate, 4U catalase, 50 mM sodium phosphate pH 7.4, and 4,000× diluted MAO-N lysate. The reactions were set up as follows: (i) all the reaction components, except for catalase and MAO-N, were mixed in a single solution and pH adjusted to 7.4 using H₃PO₄. Catalase was added, and the resultant solution (90 μL) was then aliquoted into each well of the 96-well plates, and (ii) diluted MAO-N lysate (10 μL of 400×) was then added into the wells to initiate the reaction. The reaction plate was heat-sealed with a foil seal and incubated at 30° C. with 600 rpm shaking for 16-18 hours. The reactions were quenched with 100 μL of a 1:1 mixture of acetonitrile in 20 mMv ammonium acetate. The quenched reactions were shaken for 3 min on a tabletop shaker followed by centrifugation at 4,000 rpm for 10 mins at 4° C. to pellet any precipitate. Supernatant (90 μL) was then transferred into a 96-well round bottom plate. The samples were analyzed according to the RP-HPLC analytical method in Table 4.1.

TABLE 8.1
HTP MAO Activity for the Production of Compound (2) Relative to
SEQ ID NO: 414

| | | Product Area |
|---|---|---|
| | | Fold |
| | | Improvement |
| SEQ ID | | (Relative to |
| NO: | Amino Acid Differences | SEQ ID |
| (nt/aa) | (Relative to SEQ ID NO: 414) | NO: 414)¹ |
| 999/1000 | E145L/L149M/E198D/R260S/A266L/ | +++ |
| | C338G/C347N | |
| 1001/1002 | N113G/P117K/D146A/L149M/R260S/ | +++ |
| | W262F | |
| 1003/1004 | P87G/E145L/L149M/E198D/C447A | +++ |
| 1005/1006 | L149M/E198D/I246M/R260S/A266L/C421S | +++ |
| 1007/1008 | P117K/E145L/L149M/N284D/C447A | +++ |
| 1009/1010 | D146A/L149M/R260S/W262F/C347N | +++ |
| 1011/1012 | P87L/L149M/R260S/W262F/A266L/C381A | +++ |
| 1013/1014 | P117K/E145L/D146A/W262F/N284D | +++ |
| 1015/1016 | M242I/I246M/C338G/C347N | ++ |
| 1017/1018 | N113G/E145L/D146A/L149M/M242I/ | ++ |
| | C447A | |
| 1019/1020 | P117K/E198D/W262F | ++ |
| 1021/1022 | P117K/I246M/R260S/W262F/C381A | ++ |
| 1023/1024 | P117K/M242I/I246M/R260S/A266L/ | ++ |
| | N284D/C381A | |
| 1025/1026 | E145L/D146A/M242I/R260S/W262F/ | ++ |
| | C338G/C347N/C447A | |
| 1027/1028 | E145L/D146A/L149M/R260S/W262F/ | ++ |
| | C338G/C347N | |
| 1029/1030 | P117K/E198D | ++ |
| 1031/1032 | P117K/I246M/N284D | ++ |
| 1033/1034 | N113G/M242I/C347N | ++ |
| 1035/1036 | C347N/C381A | ++ |
| 1037/1038 | P117K/M242I/R260S/W262F | ++ |
| 1039/1040 | P87G/N113G/E145L/E198D/M242I/ | ++ |
| | I246M/W262F/A266L | |
| 1041/1042 | P117K/L149M | + |
| 1043/1044 | P87L/N284D/C338G/C347N | + |
| 1045/1046 | E145L/L149M/R260S/A266L/C338G/ | + |
| | C347N/C381A/C447A | |
| 1047/1048 | I246M | + |
| 1049/1050 | C447A | + |
| 1051/1052 | E198D/C447A | + |
| 1053/1054 | P87G/L149M/M242I/R260S/A266L | + |
| 1055/1056 | M242I/C381A | + |
| 1057/1058 | M242I/R260S/W262F/C338G | + |
| 1059/1060 | N113G/P117K/A266L/C381A | + |
| 1061/1062 | P87G/P117K/L149M/M242I | + |
| 1063/1064 | N113G/P117K/C447A | + |
| 1065/1066 | P117K/A266L | + |
| 1067/1068 | W262F | + |
| 1069/1070 | P87L/M242I/I246M/R260S/W262F/ | + |
| | A266L/C338G | |
| 1071/1072 | N113G/M242I/C381A | + |
| 1073/1074 | M242I | + |
| 1075/1076 | P87L/P117K/M242I/R260S/A266L | + |
| 1077/1078 | R260S/A266L | + |
| 1079/1080 | R260S/W262F/C277T/N284D/C447A | + |
| 1081/1082 | P87L/E198D | + |
| 1083/1084 | M242I/I246M | + |

Levels of increased activity were determined relative to the reference polypeptide of SEQ ID NO: 414 and defined as follows: ″+″ .95 to 1.61, ″++″ > 1.61, ″+++″ > 2.62

### Example 9

### Improvements in Activity in MAO Relative to SEQ ID NO: 414 in Shake Flask Validation

Reactions were performed in a 96-well format in 2 mL deep-well plates, with 100 μL total volume. Reactions included 12.7 g/L (75 mMv) 6,6-dimethyl-3-azabicyclo[3.1.0]hexane acetate, 4U catalase, 50 mM sodium phosphate pH 7.4, and 0.01 g/L MAO-N SFP. The reactions were set up as follows: (i) all the reaction components, except for catalase and MAO-N, were mixed in a single solution and pH adjusted to 7.4 using H₃PO₄. Catalase was added, and the resultant solution (90 μL) was then aliquoted into each well of the 96-well plates, and (ii) diluted MAO-N SFP (10 μL of 0.1 g/L) was then added into the wells to initiate the reaction. The reaction plate was heat-sealed with a foil seal and incubated at 30° C. with 600 rpm shaking for 16-18 hours. The reactions were quenched with 100 μL of a 1:1 mixture of acetonitrile in 20 mMv ammonium acetate. The quenched reactions were shaken for 3 min on a tabletop shaker followed by centrifugation at 4,000 rpm for 10 mins at 4° C. to pellet any precipitate. Supernatant (90 μL) was then transferred into a 96-well round bottom plate. The samples were analyzed according to the RP-HPLC analytical method in Table 4.1.

TABLE 9.1
MAO Activity for the Production of Compound (2) Relative to
SEQ ID NO: 414

| | | Product Area |
|---|---|---|
| | | Fold |
| | | Improvement |
| SEQ ID | | (Relative to |
| NO: | Amino Acid Differences | SEQ ID |
| (nt/aa) | (Relative to SEQ ID NO: 414) | NO: 414)¹ |
| 1005/1006 | L149M/E198D/I246M/R260S/A266L/C421S | +++ |
| 1011/1012 | P87L/L149M/R260S/W262F/A266L/C381A | ++ |
| 1001/1002 | N113G/P117K/D146A/L149M/R260S/W262F | ++ |
| 1009/1010 | D146A/L149M/R260S/W262F/C347N | + |
| 1003/1004 | P87G/E145L/L149M/E198D/C447A | + |
| 1007/1008 | P117K/E145L/L149M/N284D/C447A | + |
| 999/1000 | E145L/L149M/E198D/R260S/A266L/ | + |
| | C338G/C347N | |

Levels of increased activity were determined relative to the reference polypeptide of SEQ ID NO: 414 and defined as follows: ″+″ .89 to 1.36, ″++″ > 1.36, ″+++″ > 1.53

### Example 10

### Validation of MAO Activity in Scaled-Down Process Testing of Fermentation Powders

To a 250-mL 3-neck flask fitted with an overhead stirrer at 300 rpm was added 600 mL of Milli-Q water at about 25° C. and 0.6 mL (approximately 0.5 g; approximately 4.5 mmol) of 6,6-dimethyl-3-azabicyclo[3.1.0]hexane to give a homogeneous solution with pH of 11.8. Na₂S₂O₅ (˜0.5 g) added portion-wise until pH 7.5 was reached. To the colorless solution was added 150 μL of Antifoam-204 (Sigma catalog number A-6226) and 300 μL of bovine catalase solution (20 g/L) to give a colorless solution at pH 7.5. To this solution was added monoamine oxidase powder (produced by fermentation in BL21), furnishing a yellow solution. An air sparging probe was inserted (airflow rate controlled using a mass flow controller at 60 mL/min), and the pH of the reaction mixture began to drop immediately. The pH was maintained via feedback-controlled dosing of 2 N NaOH. After approximately 30 minutes, the pH remained stable, and no further base dosing occurred. After an additional 10 minutes (40 minutes total), 75 mL of 6,6-dimethyl-3-azabicyclo[3.1.0]hexane NaHSO₃ solution (prepared by adding 13.05 g of 6,6-dimethyl-3-azabicyclo[3.1.0]hexane to 82.5 mL of dH₂O and enough Na₂S₂O₅ to reach pH 7.2, about 15g) was added to the reaction at a rate of 3.5 mL/h. The pH of the reaction mixture began to drop immediately, and dosing of 2 N NaOH resumed. The addition of the solution of 6,6-dimethyl-3-azabicyclo[3.1.0]hexane and NaHSO₃ was completed after 22 hours (˜23 hours of total reaction time). A 150 μL aliquot was taken and quenched with 150 μL of 8 N NaOH (to break the aminosulfonate to the free imine) and extracted with 750 μL of CDCl₃. ¹H-NMR analysis of the CDCl₃ extract was used to monitor conversion of the substrate to azabicyclo[3.1.0]hex-2-ene.

TABLE 10.1
Fermentation Powder
Testing in Scaled-Down Process
Reactions

| SEQ ID | Wt % | Percent | |
|---|---|---|---|
| NO: | Powder | Imine | St. Dev. |
| 5/6 | 2 | 75 | 1.4 |
| 413/414 | 2 | 89 | 1.4 |
| 435/436 | 2 | 83 | 2.8 |
| 5/6 | 3 | 86 | 2.1 |
| 413/414 | 3 | 90 | 2.8 |
| 435/436 | 3 | 86 | 2.8 |

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the spirit and scope of the invention(s).

What is claimed is:

1. An engineered monoamine oxidase comprising a polypeptide sequence comprising at least 80%, 85%, 86%, 87%, 88%, 89%, 90%, 91%, 92%, 93%, 94%, 95%, 96%, 97%, 98%, 99% sequence identity to SEQ ID NOs: 6 and/or 414, or a functional fragment thereof, wherein said engineered monoamine oxidase polypeptide comprises at least one substitution or substitution set in said polypeptide sequence, and wherein the amino acid positions of said polypeptide sequence are numbered with reference to SEQ ID NOs: 6 and/or 414.

2. The engineered monoamine oxidase polypeptide of claim 1, wherein said polypeptide sequence comprises at least 80%, 85%, 86%, 87%, 88%, 89%, 90%, 91%, 92%, 93%, 94%, 95%, 96%, 97%, 98%, 99% sequence identity to SEQ ID NO: 6, wherein said engineered monoamine oxidase polypeptide comprises at least one substitution at one or more positions selected from 429, 149, 198, 246, 260, 266, and 421, and wherein the amino acid positions of said polypeptide sequence are numbered with reference to SEQ ID NO: 6.

3. The engineered monoamine oxidase polypeptide of claim 1, wherein said polypeptide sequence comprises at least 80%, 85%, 86%, 87%, 88%, 89%, 90%, 91%, 92%, 93%, 94%, 95%, 96%, 97%, 98%, 99% sequence identity to SEQ ID NO: 6, wherein said engineered monoamine oxidase polypeptide comprises at least one substitution selected from 429G, 149M, 198D, 246M, 260S, 266L, and 421S, and wherein the amino acid positions of said polypeptide sequence are numbered with reference to SEQ ID NO: 6.

4. The engineered monoamine oxidase polypeptide of claim 1, wherein said polypeptide sequence comprises at least 80%, 85%, 86%, 87%, 88%, 89%, 90%, 91%, 92%, 93%, 94%, 95%, 96%, 97%, 98%, 99% sequence identity to SEQ ID NO: 6, wherein said engineered monoamine oxidase polypeptide comprises at least one substitution selected from A429G, L149M, E198D, I246M, R260S, A266L, and C421S, wherein the amino acid positions of said polypeptide sequence are numbered with reference to SEQ ID NO: 6.

5. The engineered monoamine oxidase polypeptide of claim 1, wherein said polypeptide sequence comprises at least 80%, 85%, 86%, 87%, 88%, 89%, 90%, 91%, 92%, 93%, 94%, 95%, 96%, 97%, 98%, 99% sequence identity to SEQ ID NO: 6, wherein said engineered monoamine oxidase polypeptide comprises at least one substitution or substitution set in said polypeptide sequence at one or more positions selected from 13, 32/65/135/246/284, 32/135/284, 32/284, 47/197, 55/347/348, 61, 65/135, 65/190, 65/246, 65/284, 79, 80, 82, 85, 87, 87/185, 93, 100, 100/144/421/447, 100/144/447, 104, 107, 113, 114, 115, 117, 127, 135, 136, 144, 144/447, 145, 146, 147, 149, 149/157, 153, 155, 161, 162, 166, 167, 169, 170, 174, 177, 181, 184, 185, 187/242, 193, 196, 198, 199, 208, 211, 218, 219, 219/332, 221, 229, 232, 234, 238, 239, 240, 242, 246, 250, 252, 254, 260, 262/266/421, 262/421/447, 266/447, 277, 277/347, 284, 290, 292, 294, 298, 300, 306, 310, 323, 332, 338, 347, 349, 359, 361, 366, 381, 385, 386, 387, 392, 393, 400, 407, 411/445, 413, 418, 421, 425, 429, 433, 434, 441, 444, 445, 447, 449, 452, 475, and 483, and wherein the amino acid positions of said polypeptide sequence are numbered with reference to SEQ ID NO: 6.

6. The engineered monoamine oxidase polypeptide of claim 1, wherein said polypeptide sequence comprises at least 80%, 85%, 86%, 87%, 88%, 89%, 90%, 91%, 92%, 93%, 94%, 95%, 96%, 97%, 98%, 99% sequence identity to SEQ ID NO: 6, wherein said engineered monoamine oxidase polypeptide comprises at least one substitution or substitution set in said polypeptide sequence at one or more positions selected from 79, 80, 87, 146, 149, 219/332, 238, 260, 262/266/421, 338, and 429, and wherein the amino acid positions of said polypeptide sequence are numbered with reference to SEQ ID NO: 6.

7. The engineered monoamine oxidase polypeptide of claim 1, wherein said polypeptide sequence comprises at least 80%, 85%, 86%, 87%, 88%, 89%, 90%, 91%, 92%, 93%, 94%, 95%, 96%, 97%, 98%, 99% sequence identity to SEQ ID NO: 6, wherein said engineered monoamine oxidase polypeptide comprises at least one substitution or substitution set in said polypeptide sequence at one or more positions selected from 79, 79/80, 79/80/332/429, 79/93, 79/93/238/386/429, 79/93/386, 79/183/284/332, 79/238, 79/238/332/429, 79/238/429, 80, 80/183/284/332/381/386/429, 80/188/190/332/386, 80/237/332, 80/238/332, 80/238/386/429, 80/284, 80/284/386, 80/332, 80/332/381/386, 80/332/386, 80/381, 80/386, 80/386/429, 80/429, 82, 82/87, 82/87/113, 82/87/113/117/218, 82/87/113/117/219, 82/87/113/117/219/418, 82/87/113/117/260/338, 82/87/113/117/338/418, 82/87/113/421, 82/87/117, 82/87/117/218/421, 82/87/117/260/338/421, 82/87/117/338/418, 82/87/218, 82/87/218/219/338, 82/87/218/219/421, 82/87/218/338, 82/87/219, 82/87/219/421, 82/87/338, 82/87/338/418, 82/87/338/421, 82/113/218/338, 82/113/218/338/418, 82/113/219/338, 82/113/338, 82/218/219/421, 82/260/338/421, 82/338/418/421, 82/418, 87, 87/113, 87/113/117/219/338/418/421, 87/113/117/260/338/421, 87/113/117/418/421, 87/113/218/219/260/338, 87/113/219/421, 87/113/260, 87/117, 87/117/218/219, 87/117/219/310/338, 87/218, 87/218/219, 87/218/219/338, 87/218/418, 87/219, 87/219/338, 87/219/418, 87/260, 87/310, 87/310/338, 87/338, 87/418, 87/421, 93/184/429, 93/238, 93/238/284, 93/238/386, 93/238/386/429, 100, 100/143/145/146/149/198/242/347/348/445/447, 100/143/145/149/347/447, 100/145/146/149, 100/145/146/149/154/348/445, 100/145/146/149/198, 100/145/146/149/198/242/250/252/347/348/447, 100/145/146/149/205/347/445/447, 100/145/146/149/242/445, 100/145/146/149/250/277/347/348/447, 100/145/149/154/246/277, 100/145/149/198/242/246/277/348/447, 100/145/149/198/242/246/445/447, 100/145/149/198/242/277/347/348/447, 100/145/149/198/252/348/445, 100/145/149/205, 100/145/149/242/246/277/347/445, 100/145/149/242/246/347/348/445/447, 100/145/149/242/445/447, 100/145/149/246/252/277/347/445/447, 100/145/149/246/347/445, 100/145/149/277/348/445, 100/145/149/347/445/447, 100/145/198/242/246/348, 100/145/198/277/348/445, 100/145/198/300, 100/145/205/246/277/348/445, 100/145/205/277/348, 100/145/242/246/277/445/447, 100/145/246/347/348/445, 100/145/277/347, 100/145/348, 100/146/149/198/242/246/277/347/348/445/447, 100/146/198/347/348/447, 100/146/198/445, 100/146/242/277/447, 100/146/250/447, 100/146/347/348/445/447, 100/149/198, 100/149/242/246/277/347/348/445, 100/149/242/246/277/347/348/447, 100/149/242/347/348/447, 100/149/246/277/348/445, 100/149/246/447, 100/149/250/347/447, 100/198/205/348, 100/198/277/347/348, 100/198/347/348, 100/198/347/348/445, 100/198/347/445, 100/198/447, 100/242/246/277, 100/242/246/347, 100/242/277/348, 100/242/347/348/445, 100/242/347/348/445/447, 100/242/348/445, 100/246/277/348/445, 100/277/347/445/447, 100/277/348, 100/277/348/447, 100/347/348/445, 100/347/447, 100/445/447, 110/338/418, 113/218/338/421, 113/338, 113/338/421, 117/219, 117/260, 121/198/347/445/447, 145/146/149/154/246/277/347/348, 145/146/149/198/242/252/277/347, 145/146/149/198/277/445/447, 145/146/149/205/348/447, 145/146/149/250/277/347/348/447, 145/146/149/277/347, 145/146/149/347/348, 145/146/198/205/242/277/348/447, 145/146/198/445/447, 145/146/246/277/447, 145/146/246/347/348/447, 145/146/277/347, 145/146/347/447, 145/149/198/205/348, 145/149/198/242/246/277/347/447, 145/149/198/242/246/348, 145/149/198/242/277/347, 145/149/198/246/447, 145/149/198/347/447, 145/149/205/242/347/348/445, 145/149/205/246/250/252, 145/149/205/277/445, 145/149/242, 145/149/242/348, 145/149/246, 145/149/246/347/445, 145/149/246/447, 145/149/347/348/445, 145/149/347/447, 145/149/447, 145/155/445/447, 145/198/205/348, 145/246, 145/246/252/347/447, 145/250/252/348, 145/252/348/447, 145/255, 145/277/347/445, 145/277/348/447, 145/347/348/445/447/491, 145/445/447, 146/149/151/246/348, 146/149/242/246/445, 146/149/246/252/447, 146/149/277/348/447, 146/198/246/277, 146/198/277/348/447, 146/242, 146/242/246/277/347/348/445/447, 146/246/277/348/445/447, 146/277, 146/277/347/348, 146/277/347/445/447, 146/277/445/447, 146/347/348/447, 146/348, 146/445/447, 149, 149/198/277/347/348, 149/198/348, 149/198/445, 149/242/246, 149/242/246/348/445, 149/347/348/445/447, 149/445, 184, 184/284, 184/284/429, 184/386, 198/242/348, 198/246, 198/250, 198/277/348/447, 198/445, 205, 205/246/347, 205/252/347/445/447, 205/347/348/445/447, 218, 218/260/338, 219, 219/338/418, 219/421, 238, 238/242/284, 238/284/386/429, 238/332/429, 238/381/429, 238/386, 238/429, 242, 242/277/347/348, 242/284/381/429, 242/348, 242/445/447, 246, 246/277/348/447, 246/445/447, 260, 260/338, 260/338/418/421, 277/347/348/445, 277/347/447, 284, 284/332, 284/386, 284/386/429, 310/338/418, 310/418/421, 332, 332/386, 332/429, 338, 338/418, 347/348/445, 347/348/447, 348, 348/445/447, 348/447, 386, 386/429, 418, 429, and 447, and wherein the amino acid positions of said polypeptide sequence are numbered with reference to SEQ ID NO: 6.

8. The engineered monoamine oxidase polypeptide of claim 1, wherein said polypeptide sequence comprises at least 80%, 85%, 86%, 87%, 88%, 89%, 90%, 91%, 92%, 93%, 94%, 95%, 96%, 97%, 98%, 99% sequence identity to SEQ ID NO: 6, wherein said engineered monoamine oxidase polypeptide comprises at least one substitution or substitution set in said polypeptide sequence at one or more positions selected from 80/429, 87/113/117/260/338/421, 87/421, 100/145/146/149/198/242/250/252/347/348/447, 100/145/149/198/242/277/347/348/447, 110/338/418, 145/149/205/246/250/252, 145/246/252/347/447, 145/252/348/447, 238/386/429, and 242/284/381/429, and wherein the amino acid positions of said polypeptide sequence are numbered with reference to SEQ ID NO: 6.

9. The engineered monoamine oxidase polypeptide of claim 1, wherein said polypeptide sequence comprises at least 80%, 85%, 86%, 87%, 88%, 89%, 90%, 91%, 92%, 93%, 94%, 95%, 96%, 97%, 98%, 99% sequence identity to SEQ ID NO: 414, wherein said engineered monoamine oxidase polypeptide comprises at least one substitution or substitution set in said polypeptide sequence at one or more positions selected from 87/113/145/198/242/246/262/266, 87/117/149/242, 87/117/242/260/266, 87/145/149/198/447, 87/149/242/260/266, 87/149/260/262/266/381, 87/198, 87/242/246/260/262/266/338, 87/284/338/347, 113/117/146/149/260/262, 113/117/266/381, 113/117/447, 113/145/146/149/242/447, 113/242/347, 113/242/381, 117/145/146/262/284, 117/145/149/284/447, 117/149, 117/198, 117/198/262, 117/242/246/260/266/284/381, 117/242/260/262, 117/246/260/262/381, 117/246/284, 117/266, 145/146/149/260/262/338/347, 145/146/242/260/262/338/347/447, 145/149/198/260/266/338/347, 145/149/260/266/338/347/381/447, 146/149/260/262/347, 149/198/246/260/266/421, 198/447, 242, 242/246, 242/246/338/347, 242/260/262/338, 242/381, 246, 260/262/277/284/447, 260/266, 262, 347/381, and 447, and wherein the amino acid positions of said polypeptide sequence are numbered with reference to SEQ ID NO: 414.

10. The engineered monoamine oxidase polypeptide of claim 1, wherein said polypeptide sequence comprises at least 80%, 85%, 86%, 87%, 88%, 89%, 90%, 91%, 92%, 93%, 94%, 95%, 96%, 97%, 98%, 99% sequence identity to SEQ ID NO: 414, wherein said engineered monoamine oxidase polypeptide comprises at least one substitution or substitution set in said polypeptide sequence at one or more positions selected from 87/145/149/198/447, 87/149/260/262/266/381, 113/117/146/149/260/262, 117/145/149/284/447, 145/149/198/260/266/338/347, 146/149/260/262/347, and 149/198/246/260/266/421, and wherein the amino acid positions of said polypeptide sequence are numbered with reference to SEQ ID NO: 414.

11. The engineered monoamine oxidase polypeptide of claim 1, wherein said engineered monoamine oxidase polypeptide comprises an amino acid sequence with at least 80% sequence identity to any even-numbered sequence set forth in SEQ ID NOs: 6-1084.

12. The engineered monoamine oxidase polypeptide of claim 1, wherein said engineered monoamine oxidase polypeptide comprises a polypeptide sequence set forth in the even numbered sequences of SEQ ID NOs: 6-1084.

13. The engineered monoamine oxidase polypeptide of claim 1, wherein said engineered monoamine oxidase polypeptide comprises a polypeptide sequence that exhibits at least one improved property compared to the engineered monoamine oxidase polypeptide of SEQ ID NOs: 6 and/or 414.

14. The engineered monoamine oxidase polypeptide of claim 13, wherein said improved property comprises improved production of Compound 2

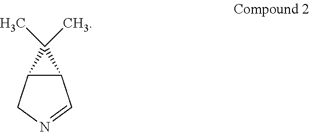

15. The engineered monoamine oxidase polypeptide of claim 13, wherein said improved property comprises reduced enzyme loading for the conversion of Compound 1 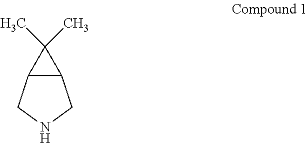
to Compound 2 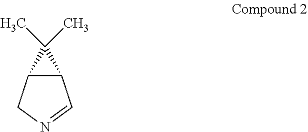
as compared to a reference sequence of SEQ ID NO: 6 or 414.

16. The engineered monoamine oxidase polypeptide of claim 13, wherein said improved property comprises 2-fold, 5-fold, 10-fold, or more activity for the conversion of Compound 1 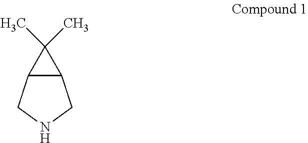
to Compound 2 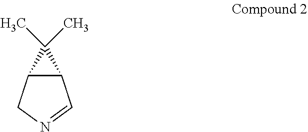
as compared to a reference sequence of SEQ ID NO: 6 or 414.

17. The engineered monoamine oxidase polypeptide of claim 1, wherein said engineered monoamine oxidase polypeptide is purified.

18. The engineered monoamine oxidase polypeptide of claim 1, wherein said engineered monoamine oxidase polypeptide is immobilized on a solid support.

19. A composition comprising at least one engineered monoamine oxidase polypeptide provided in claim 1.

20. An engineered polynucleotide encoding at least one engineered monoamine oxidase polypeptide of claim 1.

21. An engineered polynucleotide sequence encoding at least one engineered polypeptide, wherein said polynucleotide sequence comprises at least 80%, 85%, 86%, 87%, 88%, 89%, 90%, 91%, 92%, 93%, 94%, 95%, 96%, 97%, 98%, 99%, or more sequence identity to SEQ ID NOs: 5 and/or 413, wherein the polynucleotide sequence of said polypeptide comprises at least one substitution at one or more positions.

22. The engineered polynucleotide sequence of claim 20, wherein said polynucleotide sequence comprises at least 80%, 85%, 86%, 87%, 88%, 89%, 90%, 91%, 92%, 93%, 94%, 95%, 96%, 97%, 98%, 99%, or more sequence identity to SEQ ID NOs: 5 and/or 413.

23. The engineered polynucleotide sequence of claim 20, wherein said polynucleotide sequence comprises SEQ ID NOs: 5 and/or 413.

24. An engineered polynucleotide comprising any of the odd-numbered sequences set forth in SEQ ID NOs: 5-1083.

25. A vector comprising the engineered polynucleotide of claim 20.

26. The vector of claim 25, further comprising at least one control sequence.

27. A host cell comprising the vector of claim 25.

28. The host cell of claim 27, wherein said host cell produces at least one engineered monoamine oxidase polypeptide.

29. A method of producing an engineered monoamine oxidase polypeptide in a host cell, comprising culturing the host cell of claim 27, in a culture medium under suitable conditions, such that at least one engineered monoamine oxidase polypeptide is produced.

30. The method of claim 27, further comprising the step of recovering said engineered monoamine oxidase polypeptide.

31. The method of claim 29, further comprising the step of purifying said at least one engineered monoamine oxidase polypeptide.

32. A method of preparing a substantially stereomerically pure compound according to structural Formula II: 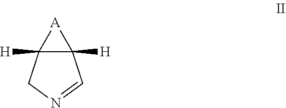
including salts and hydrates thereof, wherein:
A is O, CR¹R², —C═C—, or —CH₂—CH₂—, wherein R¹ and R² are each independently selected from —H, —COOH, —X, —NH₂, —CH₂NHC(NH)NH₂, —CX₃, —CH₃, —CH₂CH₃, and wherein X is selected from F, Cl, and Br;
the method comprising contacting an amine compound according to structural Formula I 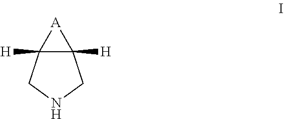
wherein A is as defined for structural Formula II, with oxygen in the presence of an engineered monoamine oxidase enzyme of claim 1 with a co-factor under conditions in which the engineered monoamine oxidase enzyme oxidizes the amine compound of structural Formula I to a corresponding imine compound of structural Formula II.

33. The method of claim 32, wherein the co-factor is non-covalently associated with the engineered monoamine oxidase enzyme, wherein the co-factor is selected from the group consisting of FAD, FMN, NAD, and NADP.

34. The method of claim 32, further comprising a component catalyzing a disproportionation of hydrogen peroxide (H₂O₂) to molecular oxygen and water, wherein the component is selected from the group consisting of Pd, Fe, and a catalase enzyme.

35. A method of preparing a mixture comprising a substantially enantiomerically pure aminosulfonate compound according to structural Formula III(a) and a substantially enantiomerically pure aminosulfonate compound according to structural Formula III(b): 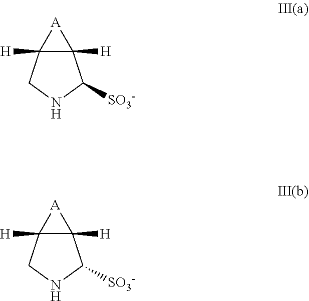
including salts and hydrates thereof, wherein:
A is O, CR¹R², —C═C—, or —CH2-CH2-, wherein R1 and R2 are each independently selected from —H, —COOH, —X, —NH2, —CH2NHC(NH)NH2, —CX3, —CH3, —CH2CH3, and wherein X is selected from F, Cl, and Br;
the method comprising contacting an amine compound according to structural Formula I 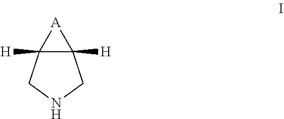
wherein A is as defined for structural Formula III with oxygen in the presence of an engineered monoamine oxidase enzyme of claim 1 associated with a co-factor and with bisulfite under conditions which yield the mixture comprising a substantially enantiomerically aminosulfonate compound according to structural Formula III(a) and a substantially enantiomerically aminosulfonate compound according to structural Formula III(b).

36. The method of claim 35 in which the bisulfite is added to the reaction before, after, or simultaneously with the engineered monoamine oxidase.

37. The method of claim 35 in which the amine compound according to structural Formula I is contacted with oxygen and the engineered monoamine oxidase enzyme associated with a co-factor before addition of the bisulfite.

38. A method of preparing a substantially enantiomerically pure aminonitrile compound according to structural Formula IV(a): 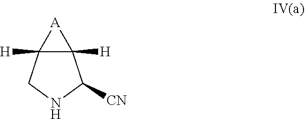
including salts and hydrates thereof, wherein:
A is O, CR¹R², —C═C—, or —CH₂—CH₂—, wherein R¹ and R² are each independently selected from —H, —COOH, —X, —NH₂, —CH₂NHC(NH)NH₂, —CX₃, —CH₃, —CH₂CH₃, and wherein X is selected from F, Cl, and Br;
the method comprising contacting an amine compound according to structural Formula I 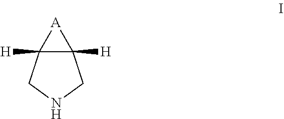
wherein A is as defined for structural Formula IV with oxygen in the presence of an engineered monoamine oxidase enzyme of claim 1 associated with a co-factor and with bisulfite under conditions which yield a mixture comprising a substantially enantiomerically pure aminosulfonate compound according to structural Formula III(a) and a substantially enantiomerically pure aminosulfonate compound according to structural Formula III(b) 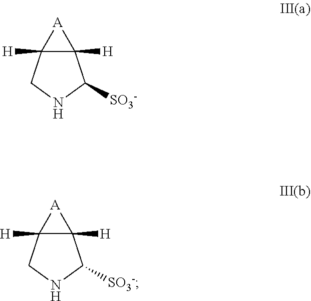
and
contacting the compounds according to structural Formulae III(a) and III(b) with cyanide under conditions which yield the substantially enantiomerically pure aminonitrile compound according to structural Formula IV(a).

39. The method of claim 38, wherein the substantially enantiomerically pure aminonitrile compound according to structural Formula IV(a) is contacted with an acid and water under conditions in which the aminonitrile compound is converted to a substantially stereomerically pure amino acid compound.

40. A method of preparing a substantially stereomerically pure amino amide compound according to structural Formula VII: 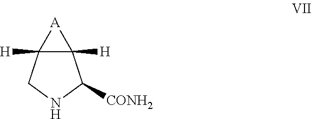
including salts thereof, wherein:
A is O, CR¹R², —C═C—, or —CH₂—CH₂—, wherein R¹ and R² are each independently selected from —H, —COOH, —X, —NH₂, —CH₂NHC(NH)NH₂, —CX₃, —CH₃, —CH₂CH₃, and wherein X is selected from F, Cl, and Br;
the method comprising contacting an amine compound according to structural Formula I 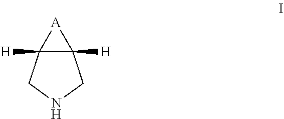
wherein A is as defined for structural Formula VII with oxygen in the presence of an engineered monoamine oxidase enzyme of claim 1 associated with a co-factor and with bisulfite under conditions which yield a mixture comprising a substantially enantiomerically pure aminosulfonate compound according to structural Formula III(a) and a substantially enantiomerically pure aminosulfonate compound according to structural Formula III(b) 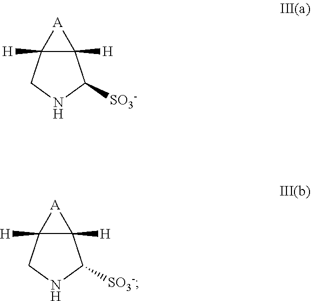
and
contacting a stereomerically pure bisulfite amine adduct compound according to structural Formula III(a), substantially stereomerically pure bisulfite amine adduct compound according to structural Formula III(b) or a mixture thereof with cyanide under conditions which yield the substantially stereomerically pure aminonitrile compound according to structural Formula IV(a) 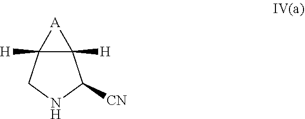
wherein A is as defined for the amino compound of structural Formula VII; and
contacting the aminonitrile compound of structural Formula IV(a) with an acid under conditions in which the aminonitrile compound is converted to the substantially stereomerically pure amino amide compound according to structural Formula VII.
